(19) United States
(12) Patent Application Publication
(10) Pub. No.: US 2021/0189361 A1
(43) Pub. Date: Jun. 24, 2021

(54) DOWNREGULATION OF SNCA EXPRESSION BY TARGETED EDITING OF DNA-METHYLATION

(71) Applicant: Duke University, Durham, NC (US)

(72) Inventors: Ornit Chiba-Falek, Durham, NC (US); Boris Kantor, Durham, NC (US)

(21) Appl. No.: 17/050,009

(22) PCT Filed: Apr. 23, 2019
(86) PCT No.: PCT/US2019/028786

§ 371 Date: Oct. 23, 2020

Related U.S. Application Data
(60) Provisional application No. 62/661,134, filed on Apr. 23, 2018.
(60) Provisional application No. 62/676,149, filed on May 24, 2018.
(60) Provisional application No. 62/789,932, filed on Jan. 8, 2019.
(60) Provisional application No. 62/824,195, filed on Mar. 26, 2019.

(51) Int. Cl. C12N 9/22; C12N 15/11; C12N 15/86; C12N 9/10

(52) U.S. Cl. CPC C12N 9/22; C12N 15/11; C12N 15/86; C12N 9/1007; A61K 38/00; C12N 2800/80; C12N 2740/15043; C07K 2319/00; C12N 2310/20

(57) ABSTRACT

Disclosed herein are Clustered Regularly Interspaced Short Palindromic Repeats (CRISPR)/CRISPR-associated (Cas) 9-based epigenome modifier compositions for epigenomic modification of a SNCA gene and methods of use thereof.

### CROSS REFERENCE TO RELATED APPLICATIONS

This application claims priority to U.S. Provisional Application No. 62/661,134, filed on Apr. 23, 2018, U.S. Provisional Application No. 62/676,149, fled on May 24, 2018, U.S. Provisional Application No. 62/789,932, fled on Jan. 8, 2019, and U.S. Provisional Application No. 62/824,195, filed on Mar. 26, 2019, the contents of each of which are hereby incorporated by reference.

### STATEMENT OF GOVERNMENT INTEREST

This invention was made with government support under federal grant number NS085011 awarded by the National Institutes of Neurological Disorders & Stroke (NIH/NINDS). The U.S. Government has certain rights to this invention.

### TECHNICAL FIELD

The present disclosure is directed to Clustered Regularly Interspaced Short Palindromic Repeats (CRISPR)/CRISPR-associated (Cas) 9-based epigenome modifier compositions for epigenomic modification of a SNCA gene and methods of use thereof.

### BACKGROUND

Parkinson's disease (PD) is the second most common neurodegenerative disorder in the world. There is no effective treatment to prevent PD or to halt its progression. The SNCA gene has been implicated as a highly significant genetic risk factor for PD. In addition, accumulating evidence suggests that elevated levels of wild type α-synuclein are causative in the pathogenesis of PD. To date, α-synuclein encoded by the SNCA gene is one of the most validated and promising therapeutic target for PD. Moreover, manipulations of SNCA levels have demonstrated a beneficial impact. However, neurotoxicity associated with robust reduction of SNCA levels has been reported studies that utilize RNA interference (RNAi) tools to directly target SNCA transcripts. As such, identification and validation of a target for achieving tight regulation of SNCA transcription that will allow maintaining normal physiological levels of α-synuclein is needed.

Several regulatory mechanisms contribute to SNCA expression levels, including genetic and epigenetic regulations. DNA methylation is an important mechanism in transcriptional regulation, and increased SNCA expression may be coincidental to demethylation of CpGs at SNCA intron 1. Furthermore, studies have shown disease related differential DNA-methylation of SNCA intron 1. Analysis of postmortem brain tissues and blood from PD patients demonstrated lower methylation levels at SNCA intron 1 compared to control donors. DNA methylation changes at SNCA intron 1 correlated with elevated SNCA-mRNA expression have also been reported in dementia with Lewy bodies (DLB) patients DNA methylation is an attractive approach for manipulation of SNCA gene expression. Moreover, DNA-methylation represents a stable epigenetic mark with a potential for long-term effects on gene expression.

Targeting specifically α-synuclein expression levels is an attractive neuroprotective strategy, and manipulations of SNCA levels have demonstrated beneficial effects. One approach to manipulate SNCA levels is through siRNA. However, the RNAi approach bears two significant shortcomings. First. RNAi does not provide a fine resolution for the knockdown where a tight-regulation is desired to achieve “physiological” level of SNCA expression. For example, AAV-vector harboring siRNA against SNCA-mRNA showed high-levels of toxicity and caused a significant loss of nigrostriatal dopaminergic neurons, as a result of robust reduction of SNCA levels in rat models. Consistently, downregulation of SNCA in MN9D cells decreased cell viability. Second, RNAi can affect the expression of genes other than their intended targets, as demonstrated by whole genome expression profiling after siRNA transfection. The role of SNCA overexpression in PD pathogenesis on the one hand, and the need to maintain normal physiological levels of α-synuclein protein on the other, emphasize the so-far unmet need to develop new therapeutic strategies targeting the regulatory mechanisms of SNCA expression. Thus, there is an unmet need to develop new therapeutic strategies targeting the regulation of SNCA expression.

### SUMMARY

The present invention is directed to a composition for epigenome modification of a SNCA gene. The composition comprises: (a)(i) a fusion protein or (a)(ii) a nucleic acid sequence encoding a fusion protein, the fusion protein comprising two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, deacetylase activity, or combination thereof, and (b)(i) at least one guide RNA (gRNA) or (b)(ii) a nucleic acid sequence encoding at least one guide gRNA, wherein the at least one gRNA targets the fusion protein to a target region within the SNCA gene.

The present invention is directed to an isolated polynucleotide encoding said composition.

The present invention is directed to a vector comprising said isolated polynucleotide.

The present invention is directed to a host cell comprising said isolated polynucleotide or said vector.

The present invention is directed to a pharmaceutical composition comprising at least one said composition, said isolated polynucleotide, said vector, said host cell, or combinations thereof.

The present invention is directed to a kit comprising at least one of said composition, said isolated polynucleotide, said vector, or combinations thereof.

The present invention is directed to a method of in vivo modulation of expression of a SNCA gene in a cell. The method comprises contacting the cell with at least one of said composition, said isolated polynucleotide, said vector, said pharmaceutical composition, or combinations thereof, in an amount sufficient to modulate expression of the gene.

The present invention is also directed to a method of in vivo modulation of expression of a SNCA gene in a subject. The method comprises contacting the subject with at least one of said composition, said isolated polynucleotide, said vector, said pharmaceutical composition, or combinations thereof, in an amount sufficient to modulate expression of the gene.

The present invention is directed to a method of treating a disease or disorder associated with elevated SNCA expression levels in a subject. The method comprises administering to the subject at least one of said composition, said isolated polynucleotide, said vector, said pharmaceutical composition, or combinations thereof. The method may comprise administering to a cell in the subject at least one of said composition, said isolated polynucleotide, said vector, said pharmaceutical composition, or combinations thereof.

The present invention is directed to a method of in vivo modulating expression of a SNCA gene in a cell. The present invention is directed to a method of in vivo modulating expression of a SNCA gene in a cell in a subject. The present invention is directed to a method of in vivo modulating expression of a SNCA gene in a subject. The method comprises contacting the cell or the subject with: (a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b)(i) at least one guide RNA (gRNA) that targets the fusion molecule to a target region within the SNCA gene or (b)(ii) a nucleic acid sequence encoding at least one gRNA that targets the fusion protein to a target region within the SNCA gene, in an amount sufficient to modulate expression of the gene.

The present invention is directed to a method of treating a disease or disorder associated with elevated SNCA expression levels in a subject. The present invention is also directed to a method of treating a disease or disorder associated with elevated SNCA expression levels in a cell in the subject. The method comprises administering to the subject or the cell in the subject: (a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b)(i) at least one guide RNA (gRNA) that targets the fusion molecule to a target region within the SNCA gene or (b)(ii) a nucleic acid sequence encoding at least one gRNA that targets the fusion molecule to a target region within the SNCA gene, in an amount sufficient to modulate expression of the gene.

The present invention is directed to a viral vector system for epigenome-editing. The viral vector system comprises: (a) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity, and (b) a nucleic acid sequence encoding at least one guide RNA (gRNA) that targets the fusion protein to a target region within the SNCA gene.

### BRIEF DESCRIPTION OF THE DRAWINGS

FIGS. 1A-1E show the design of SNCA intron 1 targeted methylation system FIG. 1A shows a schematic description of the targeted region in SNCA intron 1. Upper panel illustrates the SNCA gene structure. Lower panel depicts the sequence in intron 1 that contains CpG island [Chr4: 89.836,150-89,836.593 (GRCh38/hg38)] The gRNA sequences are marked in bold font, the PAM in S-font highlight, the CpGs are numbered and appear in upper case letters. FIG. 1B shows a schematic map of the designed vector cassette. A lentiviral vector-backbone was created to include a unique BsrGI restriction enzyme site flanked by two BsmBI sites to be used for cloning gRNAs. dCAS9-DNMT3A fused transgene was integrated into the expression cassette downstream from EFS-NC promoter. The vector also expressed puromycin-selection marker. Other regulatory elements of the vectors include a primer binding site (PBS), splice donor (SD) and splice acceptor (SA), central polypurine tract (cPPT) and PPT, Rev Response element (RRE), WPRE, and the retroviral vector packaging element, psi (ψ) signal. A human cytomegalovirus (hCMV) promoter, a core-elongation factor 1α promoter (EFS-NC), and a human U6 promoter are highlighted. FIG. 1C shows production titers of the ICLV-dCas9-DNMT3A and IDLV-dCas9-DNMT3A vectors as determined by p24gag ELISA assay. The results are recorded in copy numbers per milliliter, equating 1 ng of p2gag to 1×10⁴ viral particles (physical particles), pp.)). FIG. 1D shows a comparison between ICLV-CMV-Puro (naïve lentiviral vector and ICLV-dCas9-DNMT3A vector). The overall production and expression titers were determined by counting puromycin-resistant colonies. The bar graph data represents mean±SD from triplicate experiments. FIG. 1E shows repression of SNCA transcription by dCas9-DNMT3A in hiPSC-derived dopaminergic neurons from a PD-patient with the SNCA triplication Schematic illustration of dCas9-DNMT3A targeted CpG (not to scale) of the human SNCA locus harboring the genomic triplication. Upper panel; low level of methylation (open-lollipops) within the SNCA intron 1 region corresponds to high level of the gene expression (ON). Lower panel; gRNA-dCAS9-DNMT3A system targeting the CpGs within SNCA intron 1 to enhance methylation (closed-lollipops) resulting in downregulated expression (OFF).

FIGS. 2A-2L shows the characterization of the stable transduced SNCA-Tri MD NPCs. FIGS. 2A-2J show representative immunocytochemistry images of the SNCA-Tri MD NPCs carrying the gRNA-dCas9-DNMT3A transgene. FIGS. 2A-E show the expression of Nestin and FIGS. 2F-2J show expression of FoxA2. Scale bar=10μ. FIG. 2K and FIG. 2L show expression levels of Nestin and FoxA2, respectively, in MD NPCs. Markers were evaluated using quantitative real-time RT-PCR. The levels of mRNAs were measured by TaqMan expression assays and calculated relatively to the geometric mean of GAPDH-mRNA and PPIA-mRNA reference controls using the 2^{-ΔΔCT} method Each column represents the mean of two biological and technical replicates. The error bars represent the S.E.M.

FIG. 3 shows characterization of DNA-Methylation at the SNCA intron1 CpG island region. The methylation levels (%) of the 23 CpG sites in the SNCA intron 1 [Chr4: 89,836,150-89,836,593 (GRCh38.hg38)] in the four hiPSC-derived MD NPC lines carrying the gRNA-dCas9-DNMT3A transgenes, and the control line with the no-gRNA transgene are shown. DNA from each of the 5 cell-lines was bisulfite converted and the methylation (%) of the individual CpGs were quantitatively determined by pyrosequencing. Bars represent the mean of % methylated CpG for two independent experiments, and error bars represent the S.E.M. The significance of the reduction in methylation % was tested using the Dunnett's method and additional correction for multiple comparisons (n=23) was applied; **p<0.005, *p<0.05, two-tailed Student's t test Table 5 summarizes all methylation % values and all statistical comparisons.

FIGS. 4A-4C show SNCA-mRNA and α-synuclein protein levels in the MD NPC lines carrying the gRNA-dCas9-DNMT3A transgenes. FIG. 4A shows levels of SNCA-mRNA. Levels were assessed using quantitative RT-PCR. The SNCA-mRNA levels in the different lines were measured by TaqMan-based gene expression assay and calculated relatively to the geometric mean of GAPDH-mRNA and PPIA-mRNA reference-controls using the 2^{-ΔΔCt} method. Each bar represents the mean±S.E.M. of four biological and two technical replicates (n=8) for a particular MD NPC line. FIG. 4B shows quantification of the α-synuclein protein signals for each MD NPC line using ImageJ. Bars represents the intensity of the bands±S.E.M of two biological and technical repeats FIG. 4C shows quantification of the α-synuclein protein signal in the MD NPC line carrying the gRNA4-dCas9-DNMT3A vector and the control line with the no-gRNA vector Fifty-cells were imaged in two independent experiments (n=100 cells). Bars represent the means±S.E.M. of the intensity of α-synuclein staining in 100 cells. FIGS. 4D and 4F show representative immunocytochemistry images for the α-synuclein signal of the MD NPC lines. FIGS. 4E and 4G show representative immunocytochemistry images for the α-synuclein and Nestin double-staining signals of the MD NPC lines. Scale bar=10μ.

FIGS. 5A-5B show the effect of the gRNA4-dCas9-DNMT3A transgene on mitochondrial superoxide production and cellular viability. FIG. 5A shows mitochondrial superoxide production and FIG. 5B shows cell viability. Both were measured in SNCA-Tri MD NPC carrying the gRNA4-dCas9-DNMT3A transgene and the control MD NPC line carrying the no-gRNA transgene. Cells were treated with or without 20 μM Rotenone during the last 18 hours then, the mitochondria-associated superoxide production was determined using the MitoSox assay (FIG. 5A), and the cellular viability by the resazurin assay (FIG. 5B). Bars represent means±S.E.M of relative fluorescent units for two technical and two biological independent experiments in 6 replicates each (n=24) **p<0.005, *p<0.05; two-tailed Student's t test.

FIG. 6 shows analysis of global DNA-methylation Global 5-mC % analysis of the hiPSC-derived MD NPC lines carrying the gRNA4-dCas9-DNMT3A and the no-gRNA dCas9-DNMT3A transgenes. Global DNA-methylation (5-mC %) of the MD NPC line carrying the gRNA4 transgene showed no statistical significant difference compared to the original untransduced hiPSC-derived MD NPC line (p:=0.97). In contrast, the line carrying the no-gRNA transgene showed a significant increase in global DNA-methylation relative to the original untransduced MD NPC line (p=0.009). Each column represents the mean of two biological and technical replicates. The error bars represent the S.E.M.

FIG. 7 shows cellular characterization of iPSC-derived MD NPC by Fluorescence-activated cell sorting (FACS). FACS profile of neural intracellular markers expressed in dopaminergic differentiation. Flow cytometric analysis for Nestin, FOXA2 are shown. Combinatorial FACS analysis of Nestin and FOXA2 for MD progenitors (83.1% double positive).

FIG. 8 shows downregulation of SNCA expression by the ICLV-dCas9-DNMT3A system in rat neuroblastoma F98 cell line SNCA-mRNA in rat F98 cell line were transduced with lentiviral vector harboring gRNA-dCas9-DNMT3A transgenes. Levels of SNCA-mRNA were assessed using quantitative real-time RT-PCR 14 days post-transduction. The levels of SNCA-mRNA in the different lines (four different gRNA were designed and used) were measured by Cyber green-based gene expression assay and calculated relatively to the geometric mean of GAPDH-mRNA and PP/A-mRNA reference controls using the 2^{-ΔΔCT} method. Each bar represents the mean of three biological replicates. The results are presented as a fold of reduction from to the naïve (untrasduced) F98 cells (lane 1; black bar). Lane 2: gRNA1; Lane 3: gRNA2 Lane 4: gRNA3 (pBK744, (SEQ ID NO: 41)); Lane 5: gRNA4; Lane 6: gRNA5. No gRNA control was used in the experiment, pBK539 (SEQ ID NO: 40). The error bars represent as the S.D.

FIG. 9A shows SNCA-mRNA in the MD NPC lines transduced with integrase-deficient lentiviral vector (DLV) carrying the gRNA-dCas9-DNMT3A transgenes. SNCAmRNA were assessed using quantitative real-time RT-PCR 7 days post-transduction. The levels of SNCA-mRNA in the different lines were measured by TaqMan based gene expression assay and calculated relatively to the geometric mean of GAPDH-mRNA and PPIA-mRNA reference controls using the 2^{-ΔΔCt} method. Each bar represents the mean of four biological and two technical replicates (n=8) for a particular MD NPC line. Lane 1-492 shows no gRNA control vector. Lane 2-500 shows gRNA-dCas9-DNMT3A vector, lane 3 shows naïve (untransduced) NDs. The error bars represent the S.E.M.

FIG. 9B shows representative images of MD NPC lines transduced with integrase-deficient lentiviral vector (DLV) carrying the gRNA-dCas9-DNMT3A transgenes. FIG. 9B shows close to 80% reduction in DLV genomes by day 7 post-transduction.

FIG. 10A shows a map of pBK539, the naïve (no gRNA-vector) (SEQ ID NO: 40) that contains a catalytic domain of DNMT3A fused to dCas9 and GFP marker separated by p2A cleavage signal.

FIG. 10B shows a map of pBK744, the (gRNA3-vector that contained gRNA targeting rat SNCA gene) (SEQ ID NO: 41) that contains a catalytic domain of DNMT3A fused to dCas9 and puromycin resistant gene separated by p2A cleavage signal.

FIG. 11 shows a map of pBK500, the lentiviral vector expression cassette containing the gRNA4 sequence (gRNA4-vector) (SEQ ID NO 38) that contains a catalytic domain of DNMT3A fused to dCas9 and puromycin resistant gene separated by p2A cleavage signal.

FIG. 12A shows a map of the naïve (no gRNA-vector) pBK492 (also known as pBK546) (SEQ ID NO: 39) that contains a catalytic domain of DNMT3A fused to dCas9.

FIG. 12B shows a more detailed map of pBK546 (also known as pBK492), the naïve (no gRNA-vector) (SEQ ID NO: 39) that contains a catalytic domain of DNMT3A fused to dCas9 and puromycin resistant gene separated by p2A cleavage signal.

FIGS. 13A-13C show SNCA-mRNA and alpha-synuclein protein levels in rats treated with vehicle or rotenone. FIG. 13A shows SNCA-mRNA levels assessed by TaqMan-based gene expression assay. FIG. 13B shows the levels of alpha-syn protein were semi-quantified by Western Blot. FIG. 13C shows relative levels of alpha-synuclein protein in SN and cerebellum. The quantification was performed using ImageJ software (Schneider et al. “NIH Image to ImageJ: 25 years of image analysis”. Nature Methods 9, 671-675, 2012).

FIG. 14 shows PSer129-alpha-synuclein and ubiquitin in brain tissues of control and rotenone-treated rats. The pSer129Syn signal was increased in rotenone-treated rats compared to the controls.

FIGS. 15A-15C show SNCA expression in rat substantia nigra following the treatments with gRNA3 (pBK744) or PBS. The animals were treated with rotenone for 5 days. FIG. 15A shows the mRNA levels. FIGS. 15B and 15C show the protein levels. The quantification shown in FIG. 16C was performed using Image) software (Schneider et al. “NIH Image to ImageJ: 25 years of image analysis”. Nature Methods 9, 671-675, 2012).

FIGS. 16A-16C show the effects of DNA-methylation mediated decrease in SNCA on DNA damage. FIG. 16A and FIG. 16B show the Olive Tail Moment (OTM) analysis of the DNA damage in cells treated with the control vector (no gRNA) or with the vector with the gRNA, respectively. FIG. 16C shows the OTM values.

FIGS. 17A-17C show the effects of DNA-methylation mediated decrease in SNCA on abnormal nuclear envelope morphology: nuclear circularity. FIG. 17A and FIG. 17B show the analysis of the nuclear circularity performed using the Lamin B1 marker in cells treated with the control vector (no gRNA) or with the vector with the gRNA4, respectively FIG. 17C shows the amount of nuclear circularity.

FIGS. 18A-18C show the effects of DNA-methylation mediated decrease in SNCA on abnormal nuclear envelope morphology: nuclear folding FIG. 18A and FIG. 18B show the analysis of the nuclear folding and bubbling using the Lamin A/C marker in cells treated with the control vector (no gRNA) or with the vector with the gRNA, respectively. FIG. 18C shows the percent folded nuclei.

FIG. 19 shows heat-shock treatment and osmotic treatment applied on the NPC cells carrying the gRNA4-dCas9-DNMT3A transgene and the no-gRNA counterpart. Analysis of the nuclear circularity following the treatments was performed using the Lamin B1 marker as described elsewhere in the application (FIG. 19B). The vector with gRNA 4 (gRNA4-dCas9-DNMT3A) showed a significant increase in the nuclear circularity comparing with the no-gRNA control vector indicating it rescued the phenotype of abnormal nuclei (FIG. 19B). Analysis of the nuclear folding following the treatments was performed using the Lamin A/C marker as described elsewhere (FIG. 19A). The vector with gRNA 4 (gRNA4-dCas9-DNMT3A) showed a significant increase in the nuclear folding comparing with the no-gRNA control vector, indicating it rescued the phenotype of abnormal nuclei (FIG. 19C). The vector with gRNA 4 (gRNA4-dCas9-DNMT3A) showed a significant increase in the resistance of the nuclei to the osmotic treatment comparing with the no-gRNA control vector, indicating it rescued the phenotype of abnormal nuclei (FIG. 19C). In this experiment, the NPCs carried triplication of the SNCA gene were incubated with NaCl at different concentrations (ranging from 0 to 1000 mM) to assess the resilience of the nuclear envelope towards the osmotic shock. The bars represent the mean of three independent experiments.

FIG. 20 shows SNCA-mRNA in the SH-SY5Y cells (human neuroblastoma cells) transduced with integrase-deficient lentiviral vector (IDLV) carrying the gRNA4-dCas9-DNMT3A (pBK500) transgenes or no-gRNA-dCas9-DNMT3A control (pBK492) SNCA mRNA were assessed using quantitative real-time RT-PCR at days: 4, 7, 9, 16, 22, 27, 29, 33, and 42 post-transduction. The levels of SNCA-mRNA in the different lines were measured by TaqMan based gene expression assay and calculated relatively to the geometric mean of GAPDH-mRNA and PPIA-mRNA reference controls using the 2^{-ΔΔCt} method. Each bar represents the mean of four biological and two technical replicates (n=8). Black bar represents pBK492; grey bar represents gRNA4-dCas9-DNMT3A (pBK500) vector. The error bars represent the S.E.M.

FIG. 21 shows characterization of DNA-Methylation at the SNCA intron1 CpG island region. The methylation levels (%) of the 23 CpG sites in the SNCA intron 1 [Chr4: 89,836,150-89,836,593 (GRCh38-hg3)] (upper image represents the CpG island of SNCA intron 1). 23 CpG is highlighted. gRNA4 laying between CpG at the position 22 and 23 is highlighted. In this experiment the SH-SY5Y cells were transduced with integrase-deficient lentiviral vector (IDLV) carrying the gRNA4-dCas9-DNMT3A (pBK500) transgenes or no-gRNA-dCas9-DNMT3A control (pBK492) The DNA methylation was measured at days 3, 16 and 29. DNA from the samples was bisulfite converted and the methylation (%) of the individual CpGs were quantitatively determined by pyrosequencing. Bars represent the mean of % methylated CpG for two independent experiments, and error bars represent the S.E.M. The significance of the reduction in methylation % was tested using the Dunnett's method and additional correction for multiple comparisons (n=23) was applied. **p<0.005, *p<0.05, two-tailed Student's t test.

### DETAILED DESCRIPTION

Described herein is a system that comprises of an all-in-one lentiviral vector for targeted epigenomic editing of the SNCA gene. The disclosed epigenome modifier compositions can be used to modify any regulatory target in a SNCA gene, such as intron 1 and intron 4 The system is based on CRISPR/deactivated-Cas9 nuclease (dCas9) fused with the catalytic domain. such as a DNA methyltransferase 3A (DNMT3A). The present disclosure provides proof of concept that manipulation of gene expression, e.g. reversing overexpression, by epigenome-editing is a valuable therapeutic strategy for neurological disorders, such as PD, that involve dysregulation of gene expression.

The CRISPR/Cas9 system provides a unique opportunity to modulate gene expression in a precise fashion. The use of epigenome-editing is an approach for gene therapy and represents new smart drugs since it is designed to target specific genes. Herein, the development and implementation of an innovative epigenome editing approach to manipulate the endogenous SNCA levels for rescuing disease related phenotypes is described. For example, applying the CRISPR/Cas9 epigenome based system in human induced pluripotent stem cells (hiPSCs)-derived neurons from a PD patient with the triplication of the SNCA locus resulted in downregulation of SNCA expression, such as downregulation of SNCA-mRNA and protein, and reversed disease related phenotypic perturbations by targeted DNA-methylation of SNCA intron 1, such as the methylation in the CpG-islands along the SNCA intron 1. The reduction in SNCA levels by the gRNA-dCas9-DMNT3A system rescued cellular disease-related phenotypes characteristics of the SNCA-triplication hiPSC-derived dopaminergic neurons, e.g. mitochondrial ROS production and cellular viability. These findings establish that DNA-hypermethylation of CpG-islands within SNCA intron 1 allows an effective and sufficient tight-downregulation of SNCA expression levels, suggesting the potential of this target sequence combined with the CRISPR/dCas9 technology as a novel epigenetic-based therapeutic approach for PD.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%. more preferably up to 5%, and more preferably still up to 1% of a given value. Alternatively. particularly with respect to biological systems or processes. the term can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

As used herein, “chimeric” can refer to a nucleic acid molecule and/or a polypeptide in which at least two components are derived from different sources (e.g., different organisms, different coding regions). Also as used herein, chimeric refers to a construct comprising a polypeptide linked to a nucleic acid.

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimize.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

“Complement” as used herein can mean 00% complementarity (fully complementary) with the comparator nucleotide sequence or it can mean less than 100% complementarity (e.g., substantial complementarity)(e.g., about 80%, 81%, 82%, 83%, 84%, 85%, 86%, 87%. 88%, 89%, 90%, 91%, 92%, 93%. 94%, 95%, 96%, 97%, 98%. 99%. and the like, complementarity). Complement can also be used in terms of a “complement” to or “complementing” a mutation.

“Epigenome modification” as used herein refers to a modification or change in one or more chromosomes that affect gene activity and expression that does not derive from a modification of the genome. An epigenome modification relates to a functionally relevant change to the genome that does not involve a change in the nucleotide sequence Epigenome modifications may include a modification to a histone, such as acetylation, methylation, phosphorylation, ubiquitination, and/or sumoylation. Epigenome modifications may include a modification to DNA, such as methylation.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes can include both coding and non-coding regions (e.g., introns, regulatory elements, promoters, enhancers, termination sequences and/or 5 and 3 untranslated regions). A gene can be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

The term “genome” as used herein includes an organism's chromosomal/nuclear genome as well as any mitochondrial, and/or plasmid genome.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

As used herein, the terms “increase,” “increasing,” “increased,” “enhance,” “enhanced,” “enhancing,” and “enhancement” (and grammatical variations thereof) describe an elevation of at least about 25%, 50%, 75%, 100%, 150%, 200%, 300%, 400%, 500% or more as compared to a control.

An “isolated” polynucleotide or an “isolated” polypeptide is a nucleotide sequence or polypeptide sequence that, by the hand of man, exists apart from its native environment and is therefore not a product of nature. In some embodiments, the polynucleotides and polypeptides of the disclosure are “isolated” An isolated polynucleotide or polypeptide can exist in a purified form that is at least partially separated from at least some of the other components of the naturally occurring organism or virus, for example, the cell or viral structural components or other polypeptides or polynucleotides commonly found associated with the polypeptide or polynucleotide. In representative embodiments, the isolated polynucleotide and/or the isolated polypeptide is at least about 1%, 5%, 10%, 20%, 30%, 40%, 50%, 60%, 70%, 80%, 90%, 95%, or more pure.

In other embodiments, an isolated polynucleotide or polypeptide can exist in a non-native environment such as, for example, a recombinant host cell. Thus, for example, with respect to nucleotide sequences. the term “isolated” means that it is separated from the chromosome and/or cell in which it naturally occurs A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the polynucleotides and their encoded polypeptides are“isolated” in that, by the hand of man, they exist apart from their native environment and therefore are not products of nature, however, in some embodiments, they can be introduced into and exist in a recombinant host cell.

“Multicistronic” or “polycistronic” as used interchangeable herein refers to a polynucleotide possessing more than one coding region to produce more than one protein from the same polynucleotide. The polycistronic polynucleotide sequence can include (internal ribosome-entry site (IRES), cleavage peptides (p2A, t2A and others), utilization of different promoters, etc.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism A “homologous” nucleic acid is a nucleotide sequence naturally associated with a host cell into which it is introduced.

“Neurodegenerative diseases” are disorders characterized by, resulting from, or resulting in the progressive loss of structure or function of neurons, including death of neurons. Neurodegenerative diseases include, for example, Alzheimer's Disease (AD), amyloidosis, amyotrophic lateral sclerosis (ALS), Parkinson's Disease (PD), Huntington's Disease, priori disease, motor neuron disease, spinocerebellar ataxia, spinal muscular atrophy, neuronal loss, cognitive defect, primary age-related tauopathy (PART)/Neurofibrillary tangle-predominant senile dementia, chronic traumatic encephalopathy including dementia pugilistica, dementia with Lewy bodies (Lewy body dementia), neuroaxonal dystrophies, and multiple system atrophy, progressive supranuclear palsy. Pick's Disease, corticobasal degeneration, some forms of frontotemporal lobar degeneration, frontotemporal dementia and parkinsonism linked to chromosome 17, Lytico-Bodig disease (Parkinson-dementia complex of Guam), ganglioglioma, gangliocytoma, meningioangiomatosis, postencephalitic parkinsonism, subacute sclerosing panencephalitis, lead encephalopathy, tuberous sclerosis, Hallervorden-Spatz disease, and lipofuscinosis “Normal gene” as used herein refers to a gene that has not undergone a change, such as a loss, gain, or exchange of genetic material. The normal gene undergoes normal gene transmission and gene expression.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

A “nuclear localization signal,” “nuclear localization sequence,” or “NLS” as used interchangeably herein refers to an amino acid sequence that “tags” a protein for import into the cell nucleus by nuclear transport. Typically, this signal consists of one or more short sequences of positively charged lysines or arginines exposed on the protein surface Different nuclear localized proteins can share the same NLS. An NLS has the opposite function of a nuclear export signal, which targets proteins out of the nucleus.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

As used herein, the term “percent sequence identity” or “percent identity” refers to the percentage of identical nucleotides in a linear polynucleotide of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, the term “polynucleotide” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “polynucleotide,” “nucleotide sequence” “nucleic acid,” “nucleic acid molecule,” and “oligonucleotide” are also used interchangeably herein to refer to a heteropolymer of nucleotides. Except as otherwise indicated, nucleic acid molecules and/or polynucleotides provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST 25.

The terms “prevent,” “preventing,” and “prevention” (and grammatical variations thereof) refer to prevention and/or delay of the onset of an infection, disease, condition and/or a clinical symptom(s) in a subject and/or a reduction in the severity of the onset of the infection, disease, condition and/or clinical symptom(s) relative to what would occur in the absence of carrying out the methods of the disclosure prior to the onset of the disease, disorder and/or clinical symptom(s).

“Promoter” as used herein means a synthetic or naturally-derived molecule which is capable of conferring. activating or enhancing expression of a nucleic acid in a cell. A promoter may comprise one or more specific transcriptional regulatory sequences to further enhance expression and/or to alter the spatial expression and/or temporal expression of same A promoter may also comprise distal enhancer or repressor elements, which may be located as much as several thousand base pairs from the start site of transcription. A promoter may be derived from sources including viral, bacterial, fungal, plants. insects, and animals A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the EFS promoter, bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter.

A “protospacer sequence” refers to the target double stranded DNA and specifically to the portion of the target DNA (e.g., or target region in the genome) that is fully or substantially complementary (and hybridizes) to the spacer sequence of the CRISPR arrays. The protospacer sequence in a Type I system is directly flanked at the 3′ end by a PAM. A spacer is designed to be complementary to the protospacer.

A “protospacer adjacent motif (PAM)” is a short motif of 2-4 base pairs present immediately 3′ or 5′ to the protospacer.

As used herein, the terms “reduce,” “reduced,” “reducing,” “reduction,” “diminish,” “suppress,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction results in no or essentially no (i.e., an insignificant amount, e.g., less than about 10% or even less than about 5%) detectable activity or amount.

As used herein “sequence identity” refers to the extent to which two optimally aligned polynucleotide or peptide sequences are invariant throughout a window of alignment of components, e.g., nucleotides or amino acids “Identity” can be readily calculated by known methods including, but not limited to, those described in. Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin. A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

“Target gene” as used herein refers to any nucleotide sequence encoding a known or putative gene product. The target gene may be a mutated gene involved in a genetic disease or disorder. The target gene may be SNCA.

“Target region” as used herein refers to the region of the target gene and/or chromosome to which the composition for epigenome modification of the target gene is designed to bind and modify.

The terms “transformation,” “transfection,” and “transduction” as used interchangeably herein refer to the introduction of a heterologous nucleic acid molecule into a cell Such introduction into a cell can be stable or transient. Thus, in some embodiments, a host cell or host organism is stably transformed with a polynucleotide of the disclosure. In other embodiments, a host cell or host organism is transiently transformed with a polynucleotide of the disclosure. “Transient transformation” in the context of a polynucleotide means that a polynucleotide is introduced into the cell and does not integrate into the genome of the cell. By “stably introducing” or “stably introduced” in the context of a polynucleotide introduced into a cell is intended that the introduced polynucleotide is stably incorporated into the genome of the cell, and thus the cell is stably transformed with the polynucleotide. “Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations “Genome” as used herein also includes the nuclear, the plasmid and the plastid genome, and therefore includes integration of the nucleic acid construct into, for example, the chloroplast or mitochondrial genome. Stable transformation as used herein can also refer to a transgene that is maintained extrachromasomally, for example, as a minichromosome or a plasmid. In some embodiments, the nucleotide sequences, constructs, expression cassettes can be expressed transiently and/or they can be stably incorporated into the genome of the host organism.

“Transgene” as used herein refers to a gene or genetic material containing a gene sequence that has been isolated from one organism and is introduced into a different organism. This non-native segment of DNA may retain the ability to produce RN A or protein in the transgenic organism, or it may alter the normal function of the transgenic organism's genetic code. The introduction of a transgene has the potential to change the phenotype of an organism.

By the terms “treat,” “treating,” or “treatment,” it is intended that the severity of the subject's disease or disorder is reduced or at least partially improved or modified and that some alleviation, mitigation or decrease in at least one clinical symptom is achieved, and/or there is a delay in the progression of the disease or disorder, and/or delay of the onset of a disease or disorder. In some embodiments, the term refers to, e.g., a decrease in the symptoms or other manifestations of the disease or disorder. In some embodiments, treatment provides a reduction in symptoms or other manifestations of the disease or disorder by at least about 5%, e.g., about 10%, 15%, 20%, 25%, 30%, 35%, 40%, 45%, 50%, 60%, 70%, 80%, 90%, 95%, or more.

“Variant” used herein with respect to a nucleic acid means (i) a portion or fragment of a referenced nucleotide sequence: (ii) the complement of a referenced nucleotide sequence or portion thereof; (iii) a nucleic acid that is substantially identical to a referenced nucleic acid or the complement thereof: or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al, J. Mol. Biol. 157:105-132 (1982) The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector can be a viral vector, bacteriophage, bacterial artificial chromosome or yeast artificial chromosome. A vector can be a DNA or RNA vector A vector can be a self-replicating extrachromosomal vector, and preferably, is a DNA plasmid.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Composition for Epigenome Modification of a SNCA Gene

The present invention is directed to compositions for epigenome modification of a SNCA gene. The epigenome modification can activate or repress expression of the SNCA gene either directly or indirectly. SNCA gene has been associated with Parkinson's disease (PD) and accumulating evidence suggests that elevated levels of wild-type SNCA are pathogenic. Epigenome modification of a regulatory region of the SNCA gene can include methylation and other epigenetic modifications. For example, DNA-methylation editing directed to the SNCA gene, specifically intron 1 or intron 4, is a potential therapeutic target for neurodegenerative disorders, such as a SNCA-related disease or disorder, for downregulation of SNCA expression and reversing disease related cellular perturbations. On the other hand, normal physiological levels of SNCA are needed to maintain neuronal function. DNA-methylation at SNCA intron 1 contributes to the regulation of SNCA transcription, and differential methylation levels at SNCA intron 1 were found between PD and controls. Intron 4 of the SNCA gene is approximately 90 kb and spans a large proportion of the overall genomic sequence of the gene. Intron 4 can be divided into sub-regions based on overlap with DNaseI hypersensitivity sites (DHS), H3K4Me3, H3K4Me1, or H3K27Ac marks, and strong RepeatMasker signals. Intron 4 is associated with Lewy body pathology in Alzheimer's disease and can be involved in SNCA expression. Thus, DNA modification, including methylation or acetylation at the SNCA intron 1 locus or intron 4 is an attractive target for fine-tuned downregulation of SNCA levels.

The composition includes, but not limited to a fusion protein, or a nucleic acid encoding a fusion protein, that can be used for epigenome modification of a SNCA gene. The fusion protein includes two heterologous polypeptide domains, wherein the first polypeptide domain includes a Clustered Regularly Interspaced Short Palindromic Repeats associated (Cas) protein and the second polypeptide domain includes a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity. In some embodiments, the fusion protein includes an amino acid sequence SEQ ID NO: 13.

In some embodiments, the composition includes a fusion protein, or a nucleic acid encoding a fusion protein, and at least one guide RNA (gRNA), or a nucleic acid encoding at least one guide RNA, which targets the fusion protein to a target region within the SNCA gene. In some embodiments, the at least one gRNA targets the fusion protein to a target region within intron 1 of the SNCA gene. In some embodiments, the composition modifies at least one CpG island region within intron 1 of the SNCA gene. The CpG island region can include CpG1, CpG2, CpG3. CpG4, CpG5, CpG6, CpG7, CpG8, CpG9, CpG10, CpG11, CpG12, CpG13, CpG14, CpG15, CpG16, CpG17, CpG18, CpG19, CpG20, CpG21, CpG22, CpG23, or a combination thereof. For example, the CpG island region can include CpG1, CpG3, CpG6, CpG7, CpG8, CpG9, CpG18, CpG19, CpG20, CpG21, CpG22, or a combination thereof. In some embodiments, the at least one gRNA targets the fusion protein to a target region within intron 4 of the SNCA gene.

In some embodiments, the second polypeptide domain includes a peptide having methyltransferase activity. In such embodiments, the fusion protein methylates at least one CpG island region within intron 1 of the SNCA gene. In some embodiments, the second polypeptide domain comprises DNA (cytosine-5)-methyltransferase 3A (DNMT3A), a functional fragment thereof, and/or a variant thereof. In some embodiments, the second polypeptide domain is fused to the C-terminus, N-terminus, or both, of the first polypeptide domain. In some embodiments, the fusion protein further comprising a nuclear localization sequence. In some embodiments, the fusion protein further comprises a linker connecting the first polypeptide domain to the second polypeptide domain. In some embodiments, the second polypeptide domain comprises an amino acid sequence of SEQ ID NO:11.

a. CRISPR System

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage Short segments of foreign DNA, called spacers, are incorporated into the genome between CRISPR repeats, and serve as a ‘memory’ of past exposures. Cas9 forms a complex with the 3′ end of the sgRNA (also referred interchangeably herein as “gRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer) By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type 11 effector system may function in alternative contexts such as eukaryotic cells. The Type 11 effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase 11 This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements. The S. pyogenes CRISPR system may have the PAM sequence for this Cas9 (SpCas9) as 5′-NRG-3′, where R is either A or G, and characterized the specificity of this system in human cells. A unique capability of the CRISPR/Cas9-based epigenome modifier and modifying system is the straightforward ability to simultaneously target multiple distinct genomic loci by co-expressing a single Cas9 protein with two or more sgRNAs. For example, the Streptococcus pyogenes Type 11 system naturally prefers to use an “NGG” sequence, where “N” can be any nucleotide, but also accepts other PAM sequences, such as “NAG” in engineered systems (Hsu et al., Nature Biotechnology (2013) doi:10.1038/nbt.2647). Similarly, the Cas9 derived from Neisseria meningitidis (NmCas9) normally has a native PAM of NNNNGATT, but has activity across a variety of PAMs, including a highly degenerate NNNNGNNN PAM (Esvelt et al. Nature Methods (2013) doi:10.1038/nmeth.2681).

An engineered form of the Type II effector system of Streptococcus pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general.

b. Cas

The composition for epigenome modification of a SNCA gene may comprise a Cas fusion protein. In some embodiments, the composition for epigenome modification of a SNCA gene may comprise a Cas9 fusion protein, in which the Cas9 protein is mutated so that the nuclease activity is inactivated, i.e., a Cas9 variant. Cas9 protein is an endonuclease that cleaves nucleic acid and is encoded by the CRISPR loci and is involved in the Type 11 CRISPR system. The Cas9 protein may be from any bacterial or archaea species, such as Streptococcus pyogenes, Streptococcus thermophiles, or Neisseria mengingitides. An inactivated Cas9 protein (“iCas9”, also referred to as “dCas9”) with no endonuclease activity has been recently targeted to genes in bacteria, yeast, and human cells by gRNAs to silence gene expression through steric hindrance. As used herein, “iCas9” and “dCas9” both refer to a Cas9 protein that has the amino acid substitutions D10A and H840A and has its nuclease activity inactivated. For example, the composition for epigenome modification of a SNCA gene may include a dCas9 of SEQ ID NO: 10.

c. Cas Fusion Protein

The composition includes a Cas fusion protein. The fusion protein can include two heterologous polypeptide domains, wherein the first polypeptide domain includes a Clustered Regularly Interspaced Short Palindromic Repeats associated (Cas) protein and the second polypeptide domain includes a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity. In some embodiments, the second polypeptide domain is fused to the C-terminus. N-terminus, or both, of the first polypeptide domain. In some embodiments, the fusion protein further comprises a nuclear localization sequence. In some embodiments, the fusion protein further comprises a linker connecting the first polypeptide domain to the second polypeptide domain. In some embodiments, the fusion protein represses transcription of the SNCA gene. In some embodiments, the fusion protein is encoded by a polynucleotide sequence comprising a polynucleotide sequence of SEQ ID NO: 14

i. Transcription Activation Activity

The second polypeptide domain may have transcription activation activity, i.e., a transactivation domain. For example, the transactivation domain may include a VP16 protein, multiple VP16 proteins, such as a VP48 domain or VP64 domain, or p65 domain of NF kappa B transcription activator activity.

ii. Transcription Repression Activity

The second polypeptide domain may have transcription repression activity. The second polypeptide domain may have a Kruppel associated box activity, such as a KRAB domain, ERF repressor domain activity, Mxi1 repressor domain activity, SID4X repressor domain activity, Mad-SID repressor domain activity or TATA box binding protein activity.

iii. Transcription Release Factor Activity

The second polypeptide domain may have transcription release factor activity. The second polypeptide domain may have eukaryotic release factor 1 (ERF1) activity or eukaryotic release factor 3 (ERF3) activity.

iv. Histone Modification Activity

The second polypeptide domain may have histone modification activity. A histone modification is a covalent post-translational modification (PTM) to histone proteins which includes methylation, phosphorylation, acetylation, ubiquitylation, and sumoylation. The PTMs made to histones can impact gene expression by altering chromatin structure or recruiting histone modifiers. Histones act to package DNA, which wraps around eight histones, into chromosomes Histone modifications are involved in biological processes such as transcriptional activation/inactivation, chromosome packaging, and DNA damage/repair. The second polypeptide domain may have histone acetyltransferase, histone deacetylase, histone demethylase, or histone methyltransferase activity.

v. Nucleic Acid Association Activity

The second polypeptide domain may have nucleic acid association activity or nucleic acid binding protein-DNA-binding domain (DBD) is an independently folded protein domain that contains at least one motif that recognizes double- or single-stranded DNA. A DBD can recognize a specific DNA sequence (a recognition sequence) or have a general affinity to DNA. A nucleic acid association region can be a helix-turn-helix region, leucine zipper region, winged helix region, winged helix-turn-helix region, helix-loop-helix region, immunoglobulin fold. B3 domain, Zinc finger, HMG-box, Wor3 domain, TAL effector DNA-binding domain.

vi. Methyltransferase Activity

The second polypeptide domain may have methyltransferase activity, which involves transferring a methyl group to DNA, RNA, protein, small molecule, cytosine or adenine. DNA methylation plays a role in modulating α-synuclein expression. Differential methylation of CpG-rich region in SNCA intron 1 was reported in PD and dementia with Lewy body (DLB) patients compared to healthy individuals, specifically, hypermethylation at CpGs were detected in PD and DLB brains. The examples herein demonstrate that direct methylation of CpGs within SNCA intron 1 is sufficient to achieve sustainable and long-term downregulation of SNCA-mRNA. Moreover, the reduction in SNCA-mRNA reversed the abnormal phenotype of the SNCA-Tri MD NPCs by increasing cell viability, improving mitochondria function, and alleviating the susceptibility of the cells induction of oxidative stress as measured by mitochondrial ROS production and improving cellular viability.

In some embodiments, the second polypeptide domain may include a DNA methyltransferase. In some embodiments, the methylase activity domain can be DNA (cytosine-5)-methyltransferase 3A (DNMT3a). DNMT3a is an enzyme that catalyzes the transfer of methyl groups to specific CpG structures in DNA. The enzyme is encoded in humans by the DNMT3A gene. In some embodiment, the second polypeptide domain can cause methylation of DNA either directly or indirectly.

vii. Demethylase Activity

The second polypeptide domain may have demethylase activity. The second polypeptide domain may include an enzyme that remove methyl (CH3-) groups from nucleic acids, proteins (in particular histones), and other molecules. Alternatively, the second polypeptide may covert the methyl group to hydroxymethylcytosine in a mechanism for demethylating DNA. The second polypeptide may catalyze this reaction. For example, the second polypeptide that catalyzes this reaction may be Ten-eleven translocation methylcytosine dioxygenase 1 (Tet) or Lysine-specific histone demethylase 1 (LSD1) In some embodiment, the second polypeptide domain can cause demethylation of DNA either directly or indirectly.

viii. Acetyltransferase Activity

The second polypeptide domain may have acetyltransferase activity. The second polypeptide domain may include an enzyme that transfers an acetyl group (CH3CO—) to a molecule. The second polypeptide domain may include a histone acetyltransferase (HAT). Histone acetyltransferases are enzymes that acetylate conserved lysine amino acids on histone proteins.

ix. Deacetylase Activity

The second polypeptide domain may have deacetylase activity. The second polypeptide domain may include an enzyme that removes acetyl (CH₃CO—) groups from molecules. The second polypeptide domain may include a histone deacetylase (HDAC), also referred to as a lysine deacetylase (KDAC). Histone deacetylases are enzymes that remove acetyl groups from lysine amino acids on histone proteins.

d. gRNA

In some embodiments, the composition includes a fusion protein, or a nucleic acid encoding a fusion protein, and at least one guide RNA (gRNA), or a nucleic acid encoding at least one guide RNA, which targets the fusion protein to a target region within the SNCA gene. The gRNA provides the targeting of a CRISPR/Cas9-based epigenome modifying system. The gRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA: tracrRNA duplex involved in the Type 11 Effector system. This duplex, which may include, for example, a 42-nucleotide crRNA and a 75-nucleotide tracrRNA, acts as a guide for the Cas9.

The gRNA may target and bind a target region of the SNCA gene. In some embodiments, the at least one gRNA targets the fusion protein to a target region within intron 1 of the SNCA gene. In some embodiments, the at least one gRNA targets the fusion protein to a target region within intron 4 of the SNCA gene. For example, the at least one gRNA may target the fusion protein to the CpG island region of intron 1 of the SNCA gene. In some embodiments. the composition modifies at least one CpG island region within intron 1 of the SNCA gene. The CpG island region can include CpG1, CpG2, CpG3, CpG4, CpG5, CpG6, CpG7, CpG8, CpG9, CpG10, GpG11, CpG2, CpG13, CpG14, CpG15, CpG16, CpG17, CpG18, CpG19, CpG20, CpG21, CpG22, CpG23, or a combination thereof. For example, the CpG island region can include CpG1, CpG3, CpG6, CpG7, CpG8, CpG9, CpG18, CpG19, CpG20, CpG21, CpG22, or a combination thereof.

In some embodiments, the at least one gRNA comprises a polynucleotide sequence of at least one of SEQ ID NO: 2, SEQ ID NO: 3, SEQ ID NO: 4, SEQ ID NO: 5, complement thereof, variant thereof, or a combination thereof. In some embodiments, the composition comprises between one and ten different gRNA molecules. In some embodiments, the system comprises two or more gRNA molecules. In some embodiments, the presently disclosed epigenome modifying system includes at least one gRNA, at least two different gRNAs, at least three different gRNAs, at least four different gRNAs, at least five different gRNAs, at least six different gRNAs, at least seven different gRNAs, at least eight different gRNAs, at least nine different gRNAs, or at least ten different gRNAs. In some embodiments, the composition comprises four different gRNAs. In some embodiments, the epigenome modifying system includes a gRNA that comprises a nucleotide sequence set forth in SEQ ID NO 2, a gRNA that comprises a nucleotide sequence set forth in SEQ ID NO: 3, a gRNA that comprises a nucleotide sequence set forth in SEQ ID NO: 4, and a gRNA that comprises a nucleotide sequence set forth in SEQ ID NO: 5.

### 3. Constructs and Plasmids

The composition for epigenome modification of a SNCA gene may comprise genetic constructs that encodes the composition. The genetic construct, such as a plasmid, may comprise a nucleic acid that encodes the composition for epigenome modification of a SNCA gene. The genetic construct may encode the cas fusion protein and/or at least one of the gRNAs. The compositions, as described above, may comprise genetic constructs that encodes a modified AAV vector or lentiviral vector and a nucleic acid sequence that encodes composition, as disclosed herein. The genetic construct, such as a recombinant plasmid or recombinant viral particle, may comprise a nucleic acid that encodes the Cas fusion protein and at least one gRNA. In some embodiments, the genetic construct may comprise a nucleic acid that encodes the Cas fusion protein and at least two different gRNAs. In some embodiments, the genetic construct may comprise a nucleic acid that encodes the Cas fusion protein and more than two different gRNAs. In some embodiments, the present disclosure includes an isolated polynucleotide encoding a disclosed composition for epigenome modification of a SNCA gene. The isolated polynucleotide may encode the Cas fusion protein and at least one gRNA. The isolated polynucleotide may comprise a polynucleotide sequence of SEQ ID NO: 14.

In some embodiments, the genetic construct may comprise a promoter that operably linked to the nucleotide sequence encoding the at least one gRNA molecule and/or a Cas fusion protein molecule. In some embodiments, the promoter is operably linked to the nucleotide sequence encoding two or more gRNA molecules and/or a Cas fusion protein molecule. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant lentivirus, recombinant adenovirus, and recombinant adenovirus associated virus. The genetic construct may be part of the genetic material in attenuated live microorganisms or recombinant microbial vectors which live in cells. The genetic constructs may comprise regulatory elements for gene expression of the coding sequences of the nucleic acid. The regulatory elements may be a promoter, an enhancer, an initiation codon, a stop codon, or a polyadenylation signal.

In certain embodiments, the genetic construct is a vector. The vector can bean Adeno-associated virus (AAV) vector or a lentiviral vector. The vector can be a plasmid. The vectors can be used for in vivo gene therapy. The vector may be recombinant. The vector may comprise heterologous nucleic acid encoding the Cas fusion protein. The vector may be useful for transfecting cells with nucleic acid encoding the Cas fusion protein, which the transformed host cell is cultured and maintained under conditions wherein expression of the Cas fusion protein takes place.

Coding sequences may be optimized for stability and high levels of expression. In some instances. codons are selected to reduce secondary structure formation of the RNA such as that formed due to intramolecular bonding.

The vector may comprise heterologous nucleic acid encoding the composition for epigenome modification of a SNCA gene and may further comprise an initiation codon, which may be upstream of the coding sequence, and a stop codon, which may be downstream of the coding sequence. The initiation and termination codon may be in frame with the coding sequence. The vector may also comprise a promoter that is operably linked to the coding sequence. The promoter that is operably linked to the coding sequence may be a promoter from simian virus 40 (SV40), a mouse mammary tumor virus (MMTV) promoter, a human immunodeficiency virus (HIV) promoter such as the bovine immunodeficiency virus (BV) long terminal repeat (LTR) promoter, a Moloney virus promoter, an avian leukosis virus (ALV) promoter, a cytomegalovirus (CMV) promoter such as the CMV immediate early promoter or hCMV, Epstein Barr virus (EBV) promoter, a EFS promoter, a U6 promoter, such as the human U6 promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a muscle or skin specific promoter, natural or synthetic. Examples of such promoters are described in US Patent Application Publication Nos. US20040175727 and US20040192593, the contents of which are incorporated herein in their entirety. Examples of muscle-specific promoters include a Spc5-12 promoter (described in US Patent Application Publication No. US 20040192593, which is incorporated by reference herein in its entirety; Hakim et al. Mol. Ther Methods Clin. Dev. (2014) 1:14002; and Lai et al. Hum Mol Genet. (2014) 23(12): 3189-3199), a MHCK7 promoter (described in Salva et al., Mol Ther. (2007) 15:320-329), a CK8 promoter (described in Park et al PLoS ONE (2015) 10(4): e0124914), and a CK8e promoter (described in Muir et al., Mol Ther. Methods Clin. Dev (2014) 1:14025). In some embodiments, the expression of the composition for epigenome modification of a SNCA gene is driven by tRNAs.

Each of the polynucleotide sequences encoding the gRNA molecule and/or Cas fusion protein molecule may each be operably linked to a promoter. The promoters that are operably linked to the gRNA molecule and/or Cas fusion protein molecule may be the same promoter. The promoters that are operably linked to the gRNA molecule and/or Cas fusion protein molecule may be different promoters. The promoter may be a constitutive promoter, an inducible promoter, a repressible promoter, or a regulatable promoter.

The vector may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, Calif.).

The vector may also comprise an enhancer upstream of the coding sequence. The enhancer may be necessary for DNA expression. The enhancer may be human actin, human myosin, human hemoglobin, human muscle creatine or a viral enhancer such as one from CMV, HA, RSV or EBV. Polynucleotide function enhancers are described in U.S. Pat. Nos. 5,593,972, 5,962,428, and WO94/016737, the contents of each are fully incorporated by reference. The vector may also comprise a mammalian origin of replication in order to maintain the vector extrachromosomally and produce multiple copies of the vector in a cell. The vector may also comprise a regulatory sequence, which may be well suited for gene expression in a mammalian or human cell into which the vector is administered. The vector may also comprise a reporter gene, such as green fluorescent protein (“GFP”) and/or a selectable marker, such as hygromycin (“Hygro”).

The vector may be expression vectors or systems to produce protein by routine techniques and readily available starting materials including Sambrook et al, Molecular Cloning and Laboratory Manual, Second Ed., Cold Spring Harbor (1989), which is incorporated fully by reference. In some embodiments the vector may comprise the nucleic acid sequence encoding the composition for epigenome modification of a SNCA gene, including the nucleic acid sequence encoding the Cas fusion protein of SEQ ID NO: 14 and the nucleic acid sequence encoding the at least one gRNA comprising the nucleic acid sequence of at least one of SEQ ID NOs: 2-5, or complement thereof.

The isolated polynucleotide or the vector comprising the isolated polynucleotide may be introduced into a host cell. Methods of introducing a nucleic acid into a host cell are known in the art, and any known method can be used to introduce a nucleic acid (e.g., an expression construct) into a cell. Suitable methods include, include e.g., viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection. DEAE-dextran mediated transfection, liposome-mediated transfection, particle gun technology, calcium phosphate precipitation, direct micro injection, nanoparticle-mediated nucleic acid delivery, and the like. In some embodiments, the composition may be introduced by mRNA delivery and ribonucleoprotein (RNP) complex delivery.

a. Lentiviral Vector

CRISPR/dCas9 systems have the potential to revolutionize the field of epigenetics by enabling direct manipulation of specific regulatory sequences and epigenetic marks. The technology offers the unprecedented opportunity to fine-tune a particular epigenetic mark and correcting disease-associated expression aberrations. However, to achieve an effective epigenome directed modifications, stable transduction of the dCas9-effector tool is often necessary, in particular, when applied to primary cells or iPSCs. Delivery platform based on lentiviral vectors (LVs) is feasible and highly efficient for CRISPR-Cas9 components due to their ability to accommodate large DNA payloads and efficiently and stably transduce a wide range of dividing and non-dividing cells. LVs also display low cytotoxicity and immunogenicity and have a minimal impact on the life cycle of the transduced cells. Herein, an optimized all-in-one lentiviral vectors was adopted for highly-efficient delivery of CRISPR/dCas9-DNMT3A components. Using this LV system, efficient transduction (hiPSC)-derived dopaminergic neurons was achieved, which resulted in an effective and targeted modification of the methylation state of the CpGs within SNCA intron 1.

In some embodiments, the vector may be a lentiviral vector. The large packaging capacity of lentiviral vectors, a commonly used method to stably deliver CRISPR/Cas9 components in vitro, can accommodate the 4.2 kb S. pyogenes Cas9, epigenetic modulator fusions, a single gRNA, and associated regulatory elements required for expression. In some embodiments, the lentiviral vector may comprise the nucleic acid sequence encoding the composition for epigenome modification of a SNCA gene, including the nucleic acid sequence encoding the Cas fusion protein of SEQ ID NO. 14 and the nucleic acid sequence encoding the at least one gRNA comprising the nucleic acid sequence of at least one of SEQ ID NOs: 2-5, or complement thereof. In some embodiments, the lentiviral vector comprises a polynucleotide sequence of SEQ ID NO 38, SEQ ID NO 41. SEQ ID NO 40. or SEQ ID NO. 39.

In some embodiments, the lentiviral vector may be a modified lentiviral vector. For example, the lentiviral vector may be modified to increase vector titer. In some embodiments, the viral vector may be an episomal integrase-deficient lentiviral vector (IDLV). The IDLV may comprise the nucleic acid sequence encoding the composition for epigenome modification of a SNCA gene, including the nucleic acid sequence encoding the Cas fusion protein of SEQ ID NO: 14 and the nucleic acid sequence encoding the at least one gRNA comprising the nucleic acid sequence of at least one of SEQ ID NOs: 2-5, or complement thereof.

Episomal integrase-deficient lentiviral vectors (IDLVs) are an ideal platform for delivery of large genetic cargos where only transient expression of the transgene is desired. IDLVs retain residual (integrase-independent and illegitimate) integration rates of ˜0.2%-0.5% (one integration event per 200-500 transduced cells), which could be further reduced by packaging a novel 3′ polypurine tract (PPT)-deleted lentiviral vector into integrase-deficient particles. While efficacious for in vitro delivery, under certain circumstances, lentiviral delivery is typically not suitable for in vivo gene regulation due to concerns for insertional mutagenesis.

In contrast, the IDLV may display lower capacity to induce off-target mutations than other lentiviral vectors.

In some embodiments, the viral vector may include an episomal integrase-competent lentiviral vector (ICLV). The ICLV may comprise the nucleic acid sequence encoding the composition for epigenome modification of a SNCA gene, including the nucleic acid sequence encoding the Cas fusion protein of SEQ ID NO: 14 and the nucleic acid sequence encoding the at least one gRNA comprising the nucleic acid sequence of at least one of SEQ ID NOs: 2-5, or complement thereof.

b. Adeno-Associated Virus Vectors

The composition may also include a different viral vector delivery system. In certain embodiments, the vector is an adeno-associated virus (AAV) vector. The AAV vector is a small virus belonging to the genus Dependovirus of the Parvoviridae family that infects humans and some other primate species. AAV vectors may be used to deliver the composition for epigenome modification of a SNCA gene using various construct configurations. For example, AAV vectors may deliver Cas fusion protein and gRNA expression cassettes on separate vectors or on the same vector. Alternatively, if the small Cas9 proteins, derived from species such as Staphylococcus aureus or Neisseria meningitidis, are used then both the Cas fusion protein and up to two gRNA expression cassettes may be combined in a single AAV vector within the 4.7 kb packaging limit

In certain embodiments, the AAV vector is a modified AAV vector. For example, the modified AAV vector may be an AAV-SASTG vector (Piacentino et al (2012) Human Gene Therapy 23:635-646). The modified AAV vector may deliver nucleases to skeletal and cardiac muscle in vivo. The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5 and AAV/SASTG vectors that efficiently transduce skeletal muscle or cardiac muscle by systemic and local delivery (Seto et al. Current Gene Therapy (2012) 12:139-151). The modified AAV vector may be AAV2i8G9 (Shen et al. J. Biol. Chem. (2013) 288:28814-28823).

### 4. Pharmaceutical Compositions

The disclosure provides for pharmaceutical compositions comprising the composition, isolated polynucleotide, vector, or host cell for epigenome modification of a SNCA gene. The pharmaceutical composition may comprise about 1 ng to about 10 mg of DNA encoding the composition, polynucleotide, vector, or host cell for epigenome modification of a SNCA gene. For example, about 1 ng to about 100 ng, about 10 ng to about 250 ng, about 50 ng to about 500 ng, about 100 ng to about 750 ng, about 500 ng to about 1 mg, about 750 ng to about 2 mg, about 1 mg to about 5 mg, 2 mg to about 6 mg, about 3 mg to about 7 mg, about 4 mg to about 8 mg, about 5 mg to about 10 mg, or any value in between. The pharmaceutical compositions according to the present invention are formulated according to the mode of administration to be used. In cases where pharmaceutical compositions are injectable pharmaceutical compositions, they are aqueous, sterile-filtered and pyrogen free. An isotonic formulation is preferably used Generally, additives for isotonicity may include sodium chloride, dextrose, mannitol, sorbitol, lactose, and any combinations of the foregoing. In some cases, isotonic solutions such as phosphate buffered saline are preferred. In some cases, the pharmaceutical compostions further comprise one or more stabilizers. Stabilizers include, but are not limited to, gelatin and albumin. In some embodiments, a vasoconstriction agent is added to the formulation.

The pharmaceutical composition containing the DNA targeting system may further comprise a pharmaceutically acceptable excipient. The pharmaceutically acceptable excipient may be functional molecules as vehicles, adjuvants, carriers, or diluents. The method of administration will dictate the type of carrier to be used. Any suitable pharmaceutically acceptable excipient for the desired method of administration may be used. The pharmaceutically acceptable excipient may be a transfection facilitating agent. The transfection facilitating agent may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate. The poly-L-glutamate may be present in the pharmaceutical composition at a concentration less than 6 mg/ml. The pharmaceutical composition may include transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

### 5. Methods of Modulating SNCA Gene Expression

The present disclosure provides for methods of in vivo modulation of expression of a SNCA gene. The method can include in vivo modulation of expression of a SNCA gene in a cell. The method can include in vivo modulation of expression of a SNA gene in a subject. The method can include administering to the cell or subject the presently disclosed composition, polynucleotide, vector, host cell, or pharmaceutical composition for epigenome modification of a SNCA gene. The method can include administering to the cell or subject a pharmaceutical composition comprising the same.

In some embodiments, the disclosure provides a method of in vivo modulating expression of a SNCA gene in a cell or a subject, the method comprising contacting the cell or subject with (a)(i) a fusion protein or (a)(ii) a nucleic acid sequence encoding a fusion protein, or any other way for co-expressing bi/poly-cistronic system (internal ribosome-entry site (IRES), cleavage peptides (p2A, t2A and others), utilization of different promoters. etc., wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, deacetylase activity, or a combination thereof; and (b)(i) at least one guide RNA (gRNA) that targets the fusion molecule to a target region within the SNCA gene or (b)(ii) a nucleic acid sequence encoding at least one gRNA that targets the fusion protein to a target region within the SNCA gene, in an amount sufficient to modulate expression of the gene. The method may comprise administering to the cell or subject any of (a)(ii) and (b)(ii), (a)(i) and (b)(i), (a)(i) and (b)(ii), or (a)(ii) and (b)(i).

In some embodiments, administration of the composition, polynucleotide, vector, host cell, or pharmaceutical composition for epigenome modification of a SNCA gene may result in reduced expression of the SNCA gene in the cell or subject. For example, the method may result in a reduction in SNCA gene expression of at least about 5%, 10%, 15%, 20%, 25%, 35%, 50%, 75%, 80%, 85%, 90%, 95%, 97%, 98%, 99%, or 100% as compared to a control. In some embodiments, the expression of SNCA gene may be reduced by at least 20%. In some embodiments, the expression of SNCA gene may be reduced by at least 90%. The method may reduce SNCA gene expression to physiological levels in a control.

In some embodiments, administration of the composition, polynucleotide, vector, host cell, or pharmaceutical composition for epigenome modification of a SNCA gene may result in a reduction in levels of α-synuclein in the cell or subject. For example, the method may result in reduction in levels of α-synuclein of at least about 5%, 10%, 15%, 20%, 25%, 35%, 50%, 75%, 80%, 85%, 90%, 95%, 97%, 98%, 99%, or 100% as compared to a control. In some embodiments, levels of α-synuclein may be reduced by at least 25%. In some embodiments, levels of α-synuclein may be reduced by at least 36%.

In some embodiments, administration of the composition, polynucleotide, vector, host cell, or pharmaceutical composition for epigenome modification of a SNCA gene may result in reduced mitochondrial superoxide production in the cell or subject. For example, the method may result in a reduction in mitochondrial superoxide production at least about 5%, 10%, 15%, 20%, 25%, 35%, 50%, 75%, 80%, 85%, 90%, 95%, 97%, 98%, 99%. or 100% as compared to a control. In some embodiments. mitochondrial superoxide production may be reduced by at least 25%. In some embodiments, administration of the composition, polynucleotide, vector, host cell, or pharmaceutical composition for epigenome modification of a SNCA gene may result in increased cell viability. For example, cell viability may be increased at least 1 fold compared to control. For example, cell viability may be increased at least 1 fold, at least 1.2 fold, at least 1.4 fold, at least 1.6 fold, at least 1.8 fold, at least 2 fold, at least 2.5 fold, at least 5 fold, or at least 10 fold compared to control. In some embodiments, cell viability may be increased at least 1.4 fold compared to control. In some embodiments, administration of the composition, polynucleotide, vector, host cell, or pharmaceutical composition for epigenome modification of a SNCA gene may result in reduced mitochondrial superoxide production and/or increased cell viability compared to control. For example, mitochondrial superoxide production may be reduced by at least 25% and/or cell viability may be increased at least 1.4 fold. In some embodiments, administration of the composition, polynucleotide, vector, host cell, or pharmaceutical composition for epigenome modification of a SNCA gene may reverse DNA damage and/or rescue aging-related abnormal nuclei, such as increasing nuclear circularity or decreasing folded nuclei.

### 6. Methods of Treating Disease

The present disclosure provides for methods of treating a disease or disorder associated with elevated SNCA gene expression. The method can include administering to the subject the presently disclosed composition, polynucleotide, vector, host cell, or pharmaceutical composition for epigenome modification of a SNCA gene. The method can include administering to a cell the presently disclosed composition, polynucleotide, vector, host cell, or pharmaceutical composition for epigenome modification of a SNCA gene. The cell may be in a subject. In some embodiments, administration of the composition, polynucleotide, vector, host cell, or pharmaceutical composition for epigenome modification of a SNCA gene may reverse DNA damage and/or rescue aging-related abnormal nuclei, such as increasing nuclear circularity or decreasing folded nuclei, thereby treating and/or ameliorating the conditions associated with the disease or disorder associated with elevated SNCA gene expression.

In some embodiments, the disclosure provides a method of treating a disease or disorder associated with elevated SNCA expression levels in a subject, the method comprising administering to the subject or a cell in the subject (a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, deacetylase activity; or a combination thereof; and (b)(i) at least one guide RNA (gRNA) that targets the fusion molecule to a target region within the SNCA gene or (ii) a nucleic acid sequence encoding at least one gRNA that targets the fusion molecule to a target region within the SNCA gene, in an amount sufficient to modulate expression of the gene. The method may comprise administering to the subject or cell in the subject any of (a)(ii) and (b)(ii), (a)(i) and (b)(i), (a)(i) and (b)(ii), or (a)(ii) and (b)(i).

The disease may or disorder may be a neurodegenerative disorder. In some embodiments, the neurodegenerative disorder is a SNCA-related disease or disorder. An SNCA-related disease or disorder may be a disease or disorder characterized by abnormal expression of SNCA gene compared to control subjects without the SNCA-related disease or disorder. In some embodiments, the SNCA-related disease or disorder is characterized by increased expression of SNCA gene compared to control. In other embodiments, the SNCA-related disease or disorder is characterized by decreased expression of SNCA gene compared to control. In some embodiments, the SNCA-related disease or disorder is a neurodegenerative disorder. The neurodegenerative disorder may be a synucleinopathy Synucleinopathies are neurodegenerative diseases characterized by the abnormal accumulation of aggregates of alpha-synuclein protein. Accumulation of aggregates may occur in neurons, nerve fibres, or glial cells. Synucleionopathies include Parkinson's disease, dementia with Lewy bodies, and multiple system atrophy. For example, the neurodegenerative disorder can be Parkinson's disease. As another example, the neurodegenerative disorder can be dementia with Lewy bodies.

### 7. Methods of Delivery

Provided herein is a method for delivering the presently disclosed composition for epigenome modification of a SNCA gene to a cell. Cells may be transfected with the herein described nucleic acid compositions. The nucleic acid compositions may be delivered via electroporation Cells may be transfected via electroporation, for example. The delivered nucleic acid molecule may be expressed in the cell, wherein the resultant protein is delivered to the surface of the cell. Electroporation methods may use BioRad Gene Pulser Xcell or Amaxa Nucleofector IIb devices. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as a cationic transfection agent. Cationic transfection agents include, but are not limited to, siLentifect™, TransFectin™, Lipofectamine™ 2000, Lipofectamine® 3000, Lipofectamine™ MessengerMAX, and Lipofectamine™ RNAiMAX. The vector-mediated gene-transfer and the associated production are outlined in Example 14.

Upon delivery of the presently disclosed genetic constructor composition to the tissue, and thereupon the vector into the cells of the mammal, the transfected cells will express the gRNA molecule(s) and the Cas fusion protein molecule. The genetic construct or composition may be administered to a mammal to alter gene expression or to re-engineer or alter the genome. The mammal may be human, non-human primate, cow, pig, sheep, goat, antelope, bison, water buffalo, bovids, deer, hedgehogs, elephants, llama, alpaca, mice, rats, or chicken, and preferably human, cow, pig, or chicken.

The genetic construct (e.g., a vector) encoding the gRNA molecule(s) and the Cas fusion protein molecule can be delivered to the mammal by DNA injection (also referred to as DNA vaccination) with and without in vivo electroporation, liposome mediated, nanoparticle facilitated, and/or recombinant vectors. The recombinant vector can be delivered by any viral mode. The viral mode can be recombinant lentivirus, recombinant adenovirus, and/or recombinant adeno-associated virus. A presently disclosed genetic construct (e.g., a vector) or a composition comprising thereof can be introduced into a cell for epigenome modification.

### 8. Routes of Administration

The presently disclosed composition, polynucleotide, vector, host cell, or pharmaceutical composition for epigenome modification of a SNCA gene can be administered to the subject or cell in a subject by any suitable route. For example, the disclosed composition, polynucleotide, vector, host cell, or pharmaceutical composition for epigenome modification of a SNCA gene can be administered to a subject or a cell in a subject by different routes including orally, parenterally, sublingually, transdermally, rectally, transmucosally, topically, via inhalation, via buccal administration, intrapleurally, intravenous, intraarterial, mitraperitoneal, subcutaneous, intramuscular, intranasal, intrathecal, and intraarticular or combinations thereof. In certain embodiments, the presently disclosed composition, polynucleotide, vector, host cell, or pharmaceutical composition for epigenome modification of a SNCA gene is administered to a subject intramuscularly, intravenously or a combination thereof. In some embodiments, the disclosed composition, polynucleotide, vector, host cell, or pharmaceutical composition for epigenome modification is administered directly to the central nervous system of the subject. For example, direct administration to the central nervous system of the subject may comprise intracranial or intraventricular injection. For veterinary use, the presently disclosed genetic constructs (e.g., vectors) or compositions may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gone guns”, or other physical methods such as electroporation (“EP”), “hydrodynamic method”, or ultrasound.

The presently disclosed composition, polynucleotide, vector, host cell, or pharmaceutical composition for epigenome modification of a SNCA gene may be delivered to the mammal by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the skeletal muscle or cardiac muscle.

### 9. Cell Types

Any of these delivery methods and/or routes of administration can be utilized with a myriad of cell types, for example, including, but not limited to eukaryotic cells or prokaryotic cells. In some embodiments, the eukaryotic cell can be any eukaryotic cell from any eukaryotic organism. Non-limiting examples of eukaryotic organisms include mammals, insects, amphibians, reptiles, birds, fish, fungi, plants, and/or nematodes. In some embodiments, the cell is a mammalian cell. In some embodiments, the cell is a human cell. In some embodiments, the cell is a neuronal cell. For example, the cell may be a midbrain dopaminergic neuron (mDA) The cell may be a basal forebrain cholinergic neuron (BFCN). In other embodiments, the cell may be a neural progenitor cell. For example, the cell may be a dopaminergic (ventral midbrain) Neural Progenitor Cell (MD NPC). The cell may comprise a mutation in the SNCA gene. For example, the cell may comprise a mutation in the SNCA gene that causes increased SNCA gene expression in the cell or subject. In some embodiments, the cell may comprise a SNCA gene triplication (SNCA-Tri), wherein the levels of SNCA are elevated compared to physiological levels in a control cell that does not have SNCA-Tri. The cell may be a human induced Pluripotent Stem Cell (hiPSC). For example, the cell may be an hiPSC derived from a patient with a disease or disorder. For example, the cell may be an hiPSC derived from a patient diagnosed or at risk of developing Parkinson's Disease. The cell may be an hiPSC derived from a patient diagnosed with or at risk of developing Dementia with Lewy Bodies.

### 10. Kits

Provided herein is a kit, which may be used for epigenome modification of a SNCA gene. The kit may comprise the disclosed composition, polynucleotide, vector, or pharmaceutical composition for epigenome modification of a SNCA gene. The kit may comprise instructions for using the disclosed composition, polynucleotide, vector, or pharmaceutical composition for epigenome modification of a SNCA gene. Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### 11. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein Having now described the present disclosure in detail, the same will be more clearly understood by reference to the following examples, which are merely Intended only to illustrate some aspects and embodiments of the disclosure, and should not be viewed as limiting to the scope of the disclosure. The disclosures of all journal references. U.S. patents, and publications referred to herein are hereby incorporated by reference in their entireties.

The present invention has multiple aspects, illustrated by the following non-limiting examples.

### Example 1

### Materials and Methods

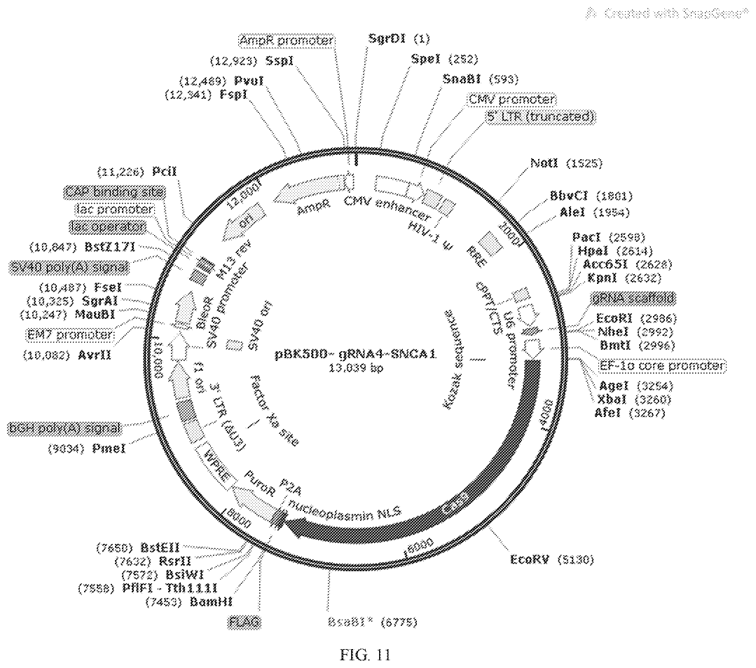

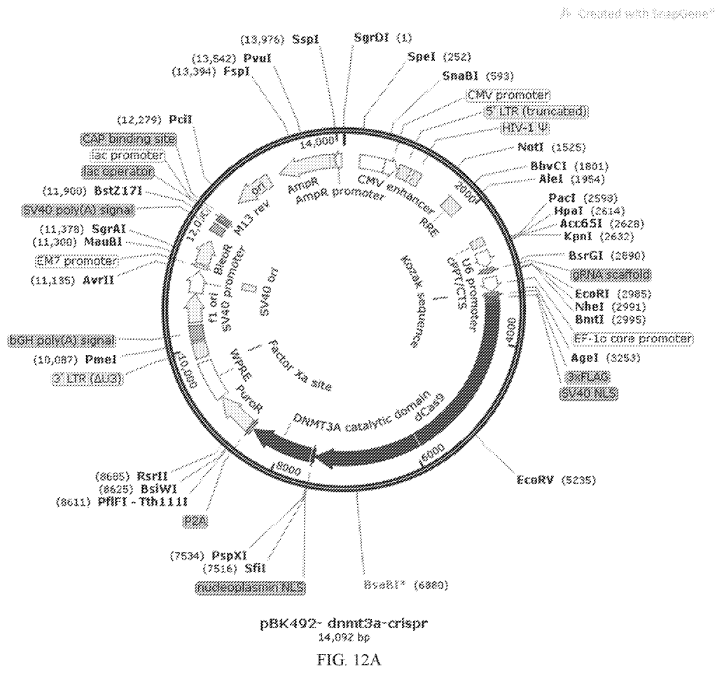

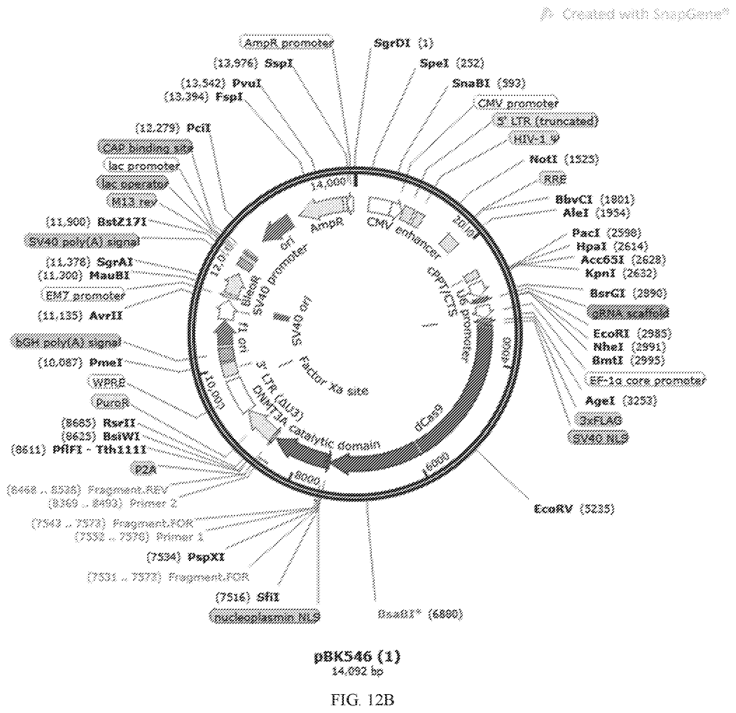

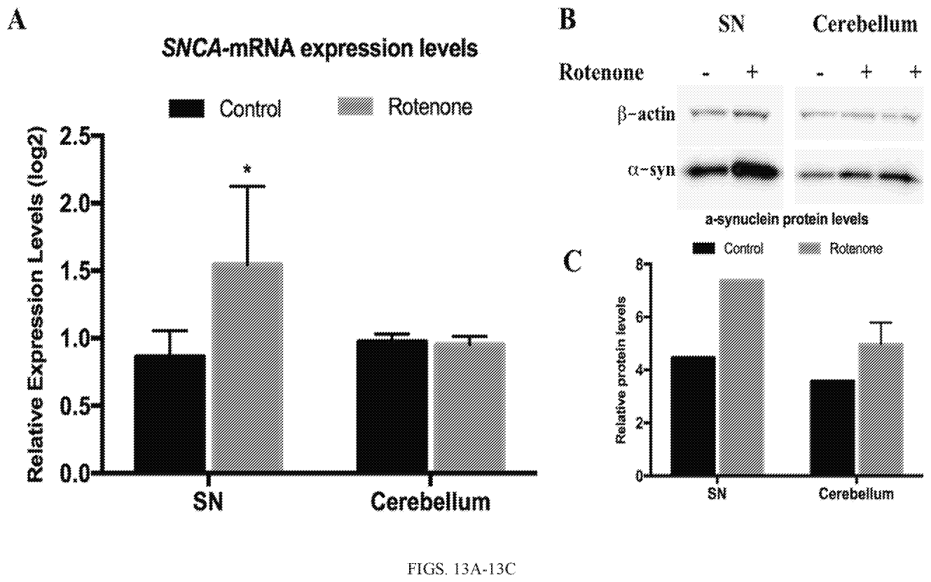

Plasmid design and construction. dCas9-DNMT3A transgene was derived from pdCas9-DNMT3A-EGFP (Addgene plasmid #71666) and cloned into pBK301 (production-optimized lentiviral vector), as follows: pBK456 plasmid was generated by cloning the dCas9 fragment digested with AgeI-BamHI restriction enzymes into pBK301 Next, DNMT3A catalytic domain was transferred from pdCas9-DNMT3A-EGFP into pBK456 by amplifying DNMT3A fragment from the plasmid with the primers containing the BamHI-restriction sites: BamHI-429/R 5′-GAGCGGATCCCCCTCCCG-3′ (SEQ ID NO: 15), BamHI-429/L5-CTCTCCACTGCCGGATCCGG-3′ (SEQ ID NO: 16). The pBK456 was then digested with BamHI restriction enzyme for the cloning, resulting in the pBK492 plasmid (no-gRNA plasmid). Next, an extra-BsmBI site located in the DNMT3A fragment was eliminated by site-directed mutagenesis to create pBK546 (SEQ ID NO. 39; see FIG. 12B). This plasmid comprised dCas9-DNMT3A-p2a-puromycin expressed from the EFS-NC promoter and gRNA-cloning site (BsmBI-BsrGI-BsmBI) located downstream of the U6 promoter. Four gRNA sequences targeting intron1-SNCA gene were used: 1) 5′-TTGTCCCTTTGGGGAGCCTA-3′ (SEQ ID NO: 2); 2) 5′-AATAATGAAATGGAAGTGCA-3′ (SEQ ID NO: 3); 3) 5′-GGAGGCTGAGAACGCCCCCT-3′ (SEQ ID NO: 4); 4) 5′-CTGCTCAGGGTAGATAGCTG-3′ (SEQ ID NO: 5). The gRNA-contained plasmids were named: pBK497/gRNA1; pBK498/gRNA2; pBK499/gRNA3; pBK500 gRNA4 (SEQ ID NO: 38; see FIG. 11), respectively. All plasmids were verified by restriction digestion analysis and Sanger sequencing. The target sequences for the gRNA sequences are shown in Table 1.

TABLE 1

| | | SEQ | Target | SEQ |
|---|---|---|---|---|
| gRNA | Sequence | ID NO: | sequence | ID NO: |
| gRNA1 | ttgtcccttt | 2 | ttgtccctttgg | 6 |
| | ggggagccta | | ggagcctaagg | |
| gRNA2 | aataatgaaa | 3 | aataatgaaatg | 7 |
| | tggaagtgca | | gaagtgcaagg | |
| gRNA3 | ggaggctgag | 4 | ggaggctgagaa | 8 |
| | aaCGccccct | | CGccccctCGg | |
| gRNA4 | ctgctcaggg | 5 | ctgctcagggta | 9 |
| | atgatagctg | | gatagctgagg | |

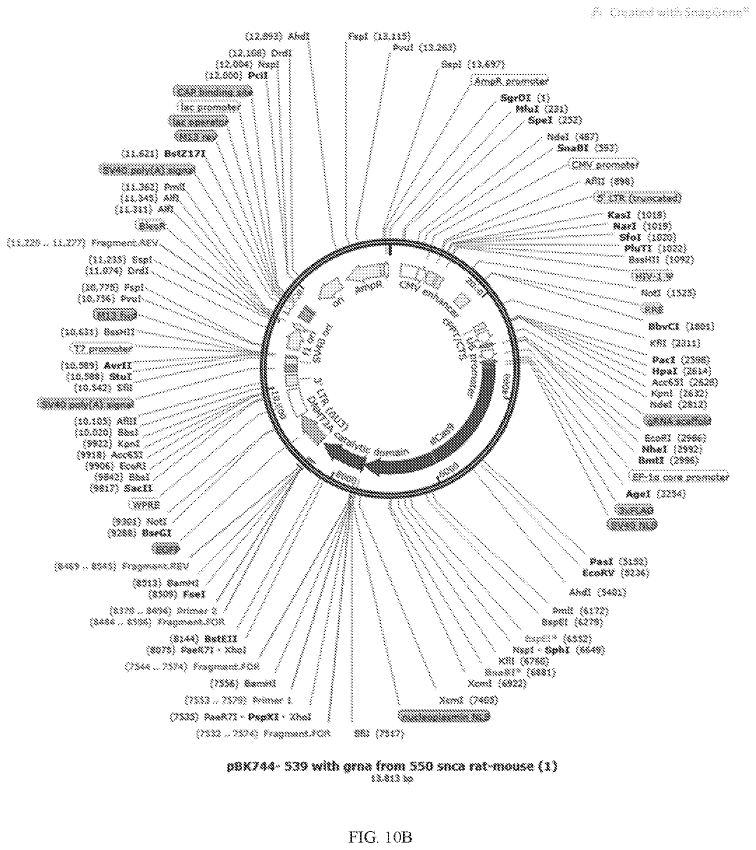

The following plasmids were created to target rat/mouse Snca-intron 1 sequences pBK539 was created to replace puromycin with GFP marker. The replacement is necessary for evaluation of the transgene expression in vivo. PBK539 (SEQ ID NO: 40: see FIG. 10A) was created as follows: the GFP fragment was derived from pBK201a (pLV-GFP) by digestion with FseI restriction. The fragment was gel-purified and cloned into pBK546 vector digested with FseI. The resulted plasmid pBK539 harbors dCas9-DNMT3A-p2a-GFP transgene. This parental plasmid was further used to create pBK744 (SEQ ID NO: 41; see FIG. 10B) To this end, the plasmid was digested with BsmBI and cloned with gRNA harbored the following sequence: 5′-TTTTTCAAGCGGAAACGCTA-3′ (SEQ ID NO. 42)

Vector production Lentiviral vectors were generated using a transient transfection protocol 15 μg vector plasmid, 10 μg psPAX2 packaging plasmid (Addgene, #12260 generated in Dr Didier Trono's lab, EPFL, Switzerland), 5 μg pMD2 G envelope plasmid (Addgene #12259, generated in Dr. Trono's lab), and 2.5 μg pRSV-Rev plasmid (Addgene #12253, generated in Dr. Trono's lab) were transfected into 293T cells. Vector particles were collected from filtered conditioned medium at 72 h post-transfection. The particles were purified using the sucrose-gradient method and concentrated>250-fold by ultracentrifugation (2 h at 20,000 rpm). Vector and viral stocks were aliquoted and stored at −80° C.

Tittering vector preparations. Titers were determined for the vectors expressed puromycin-selection marker by counting puromycin-resistant colonies and by p24^{gag}ELISA method equating 1 ng p24gag to 1×10⁴ viral particles. The multiplicity of infections (MOIs) was calculated by the ratio of the number of viral particles to the number of cells. The p24^{gag} ELISA assay was carried out as per the instructions in the HIV-1 p24 antigen capture assay kit (NIH AIDS Vaccine Program). Briefly, high-binding 96-well plates (Costar) were coated with 100 μL monoclonal anti-p24 antibody (NIH AIDS Research and Reference Reagent Program, catalog #3537) diluted 1:1500 in PBS. Coated plates were incubated at 4° C. overnight, then blocked with 200 μL 1% BSA in PBS and washed three times with 200 uL 0.05% Tween 20 in cold PBS Next, plates were incubated with 200 μL samples. inactivated by 1% Triton X-100 for 1 h at 37° C. HIV-1 standards (catalog no SP968F) were subjected to a 2-fold serial dilution and applied to the plates at a starting concentration equal to 4 ng/mL. Samples were diluted in RPMI 1640 supplemented with 0.2% Tween 20 and 1% BSA, applied to the plate and incubated at 4° C. overnight. Plates were then washed six times and incubated at 37° C. for 2 h with 100 μL polyclonal rabbit anti-p24 antibody (catalog #SP451 T), diluted 1:500 in RPMI 1640, 10% FBS, 0.25% BSA, and 2% normal mouse serum (NMS; Equitech-Bio). Plates were then washed as above and incubated at 37° C. for 1 h. with goat anti-rabbit horseradish peroxidase IgG (Santa Cruz), diluted 1:10,000 in RPMI 1640 supplemented with 5% normal goat serum (NGS; Sigma), 2% NMS, 0.25% BSA, and 0.01% Tween 20. Plates were washed as above and incubated with TMB peroxidase substrate (KPL) at room temperature for 10 min. The reaction was stopped by adding 100 uL 1 N HCL. Plates were read by Microplate Reader (The iMark™ Microplate Absorbance Reader, Bio-Rad) at 450 nm and analyzed in Excel. All experiments were performed in triplicates.

Cell culture, Neural Progenitor Cells differentiation and characterization. Human induced pluripotent stem cell (hiPSC) line from a patient with a triplication of the SNCA gene (SNCA-Tri, ND34391) was purchased from the NINDS Human Cell and Data Repository. The ND34391 cell line shows a normal karyotype. The hiPSCs were cultured under feeder-independent conditions in mTeSR™1 medium (StemCell Technologies) onto hESC-qualified Matrigel coated plates. Cells were passaged using Gentle Cell Dissociation Reagent (StemCell Technologies) according to the manufacturer's manual.

The dopaminergic neurons are the primary neuronal type affected in PD, therefore a specific protocol to differentiate the hiPSC into dopaminergic (ventral midbrain) Neural Progenitor Cells (MD NPCs) was used. The hiPSCs were differentiated into MD NPCs using an embryoid body-based protocol. biPSCs were dissociated with Accutase (StemCell Technologies) and seeded into Aggrewell 800 plates (10,000 cells per microwell; Stem Cell Technologies) in Neural Induction Medium (NIM—Stem Cell Technologies) supplemented with Y27632 (10 μM) to form Embryoid Bodies (EBs). On day 5, EBs were replated onto matrigel-coated plates in NIM On day 6, NIM was supplemented with 200 ng/mL SHH (Peprotech) leading to the formation of neural rosettes. On day 12, neural rosettes were selected with Neural Rosette Selection reagent (used per the manufacturer's instructions, StemCell Technologies) and replated in matrigel-coated plates in N2B27 medium supplemented with 3 μM CHIR99021, 2 μM SB431542, 5 μg/ml BSA. 20 ng/ml bFGF, and 20 ng/ml EGF, leading to the formation of MD NPCs. MD NPCs were passaged every two days using Accutase (StemCell Technologies). The successful differentiation was assessed by Real-Time PCR and immunocytochemistry using MD NPC-specific markers listed in Tables 2 and 3, respectively.

The stably transduced MD NPC lines carrying the different gRNA-dCas9-DNMT3A transgenes, were split every 5 days and cultured onto matrigel coated plates in puromycin selection medium. Molecular and cellular characterizations were performed after 7-14 days of culturing.

TABLE 2
TaqMan Assays used for characterization of hiPSC-derived
MD NPC cells and for SNCA-mRNA quantification

| Target | Assay ID | Marker |
|---|---|---|
| SNCA | Hs00240906 | |
| FoxA2 | Hs00232764 | MD Prog |
| Nestin | Hs04187831 | NPC |
| GAPDH | Hs99999905 | House-keeping |
| PPIA | Hs99999904 | House-keeping |

TABLE 3
Primary antibodies used for characterization of
hiPSC-derived MD NPC cells by Immunocytochemistry

| | Company | Catalog No. | Dilution | Marker |
|---|---|---|---|---|
| α-synuclein | Abcam | Ab138501 | 1:150 | α-synuclein |
| | | | | quantification |
| FOXA2 | Abcam | Ab60721 | 1:250 | MD prog |
| Nestin | Abcam | Ab18102 | 1:200 | NPC |

Transduction and puromycin-selection. MD NPCs were transduced with LV/gRNA-dCas9-DNMT3A vectors at the multiplicity of infections (MOIs)=2. Sixteen hours post-transduction the media was replaced, and at 48-hours post-transduction puromycin was applied at the final concentration of 1 ug/mL. The cells were maintained on the puromycin selection medium for 21 days to obtain the five stable MD NPC-lines that carry each of the different LV/dCas9-DNMT3A vectors.

DNA extraction, bisulfite conversion and pyrosequencing gDNA was extracted from each stably transduced cell line using DNeasy Blood and Tissue Kit (Qiagen) per manufacturers' instructions. gDNA samples (800 ng) were treated with sodium bisulfite using the Zymo EZ DNA Methylation™ Kit (Zymo Research). Pyrosequencing assays were designed using the PyroMark assay design software version 1.0.6 (Biotage: Uppsala. Sweden) for specific evaluation of the methylation status at 23 CpGs in the SNCA intron1 region [Chr4: 89,836,150-89,836,593 (GRCh38/hg38)]. Assays were validated for linearity and range on a PyroMark Q96 MD pyrosequencer using mixtures of unmethylated (U) and methylated (M) bisulfite modified DNAs in the following ratios: 100 U:0M, 75 U:25M, 50 U:50M, 25 U:75M, 0 U:100M (EpiTect Control DNA Set; Qiagen). Bisulfite modified DNA (20 ng) was added to the PyroMark PCR Master Mix (Qiagen) and subjected to PCR using the following conditions: 95° C. for 15 m, 50 cycles of 94° C. for 30 s, 56° C. for 30 s and 72° C. for 30 s with a final 10 m extension step at 72° C. Primers for amplification and sequencing are listed in Table 4. Pyrosequencing was conducted using PyroMark Gold Q96 Reagents (Qiagen) following the manufacture's protocol. Methylation values for each CpG site were calculated using Pyro Q-CpG software 1.0.9 (Biotage). Each stably transduced cell-line was analyzed in two independent experiments.

TABLE 4
Pyrosequencing assays for evaluation
of the methylation levels of the 23 CpG
at SNCA intron 1

| Primer Forward | Primer Reverse | Sequencing Primer | CpG |
|---|---|---|---|
| (5′-3′) | (5′-3′) | (5′-3′) | Covered |
| TTTTTGGGGAGTTTA | AACCTCCTTACACTTC | GGGGAGTTTAAGGAAA | 1 |
| AGGAAAGA | CATTTCAT* | GA | |
| (SEQ ID NO: 17) | (SEQ ID NO: 18) | (SEQ ID NO: 19) | |
| TGGGGAGTTTAAGGA | ACCTCCTTACACTTCC | GGTTGAGAGATTAGGT | 2, 3, 4, 5, |
| AAGAGATTT | ATTTCATT* | TGTT | 6, 7 |
| (SEQ ID NO: 20) | (SEQ ID NO: 21) | (SEQ ID NO: 22) | |
| TTGGGGAGTTTAAGG | ACCTCCTTACACTTCC | AGAGAGGATGTTTTAT | 7, 8 |
| AAAGAGAT | ATTTCATT* | G | |
| (SEQ ID NO: 23) | (SEQ ID NO: 24) | (SEQ ID NO: 25) | |
| TTTTTGGGGAGTTTA | CCTCCTTACACTTCCA | CTTACACTTCCATTTC | 9, 8 |
| AGGAAAGA* | TTTCATT | ATTAT | |
| (SEQ ID NO: 26) | (SEQ ID NO: 27) | (SEQ ID NO: 28) | |
| TGGGGAGTTTAAGGA | CCCTCAACTATCTACC | GAGTTTGGTAAATAAT | 10, 11, 12, |
| AAGAGATTT | CTAAACA* | GAA | 13, 14, 15, |
| (SEQ ID NO: 29) | (SEQ ID NO: 30) | (SEQ ID NO: 31) | 16, 17 |
| GTGTAAGGAGGTTAA | ACAACAAACCCAAATA | AGGTTAAGTTAATAGG | 17, 18, 19, |
| GTTAATAGG | TAATAATTCTAAT* | TGGTAA | 20, 21, 22 |
| (SEQ ID NO: 32) | (SEQ ID NO: 33) | (SEQ ID NO: 34) | |
| TTTTTGGGGAGTTTA | CTCAAACAAACAACAA | CTCAAACAAACAACAA | 23, 22, 21, |
| AGGAAAGA* | ACCCAAAT | ACCCAAAT | 20 |
| (SEQ ID NO: 35) | (SEQ ID NO: 36) | (SEQ ID NO: 37) | |

Primers for amplification and sequencing are listed
*indicates biotinylated primers.

RNA extraction and expression analysis. Total RNA was extracted from each stably transduced MD NPC line using TRIzol reagent (Invitrogen) followed by purification with an RNeasy kit (Qiagen) used per the manufacturer's protocol. RNA concentration was determined spectrophotometrically at 260 nm, while the quality of the purification was determined by 260 nm/280 nm ratio that showed values between 1.9 and 2.1, indicating high RNA quality. cDNA was synthesized using MultiScribe RT enzyme (Applied Biosystems) using the following conditions: 10 min at 25° C. and 120 min at 37° C.

Real-time PCR was used to quantify the levels of the MD NPC markers and SNA expression levels. Briefly, duplicates of each sample were assayed by relative quantitative real-time PCR using TaqMan expression assays and the ABI QuantStudio 7. ABI MGB probe and primer set assays (Applied Biosystems) that were used are listed in Table 2. Each cDNA (20 ng) was amplified in duplicate in at least two independent runs for two independent experiments (overall≥8 repeats), using TaqMan Universal PCR master mix reagent (Applied Biosystems) and the following conditions: 2 min at 50° C., 10 min at 95° C., 40 cycles: 15 sec at 95° C., and 1 min at 60° C. As a negative control for the specificity of the amplification, we used RNA control samples that were not converted to cDNA (no-RT) and no-cDNA-RNA samples (no-template) in each plate. No amplification product was detected in control reactions. Data were analyzed with a threshold set in the linear range of amplification. The cycle number at which any particular sample crossed that threshold (Ct) was then used to determine fold difference, whereas the geometric mean of the two control genes served as a reference for normalization. Fold difference was calculated as 2^{-ΔΔCt} (31); ΔCt=[Ct(target)−Ct (geometric mean of reference)]. ΔΔCt=[ΔCt(sample)]−[ΔCt(calibrator)]. The calibrator was a particular RNA sample, obtained from the control MD NPCs, used repeatedly in each plate for normalization within and across runs. The variation of the ΔCt values among the calibrator replicates was smaller than 10%.

Immunocytochemistry and Imaging. Prior to immunostaining, MD NPCs were plated onto Matrigel Coated Cells Imaging Coverglasses (Eppendorf, 0030742060) MD NPCs were fixed in 4% paraformaldehyde and permeabilized in 0.1% Triton-X100. Immunocytochemistry was performed as follows cells were blocked in 5% goat serum for 1 hour before incubating with primary antibodies overnight at 4° C. (Table 3). Secondary antibodies (AlexaFluor, Life Technologies) were incubated for 1 hour at room temperature Nuclei were stained with NucBlue® Fixed Cell ReadyProbes® Reagent (ThermoFisher). according to the manufacturers' instructions. Images were acquired on the Leica SP5 confocal microscope using a 40× objective. The staining was performed in two independent experiments, 50 cells were analyzed in each experiment (n=100 cells).

Western blotting. Expression levels of human α-synuclein protein in the stably transduced MD NPC lines were determined by Western blotting with the α-synuclein rabbit monoclonal antibody (ab138501, Abcam) and with mAb β-actin (Transduction Labs) for normalization Cell were scraped from the dish and homogenized in 10× volume of 50 mM Tris-HC, pH 7.5, 150 mM NaCl, 1% Nonidet P-40, in the presence of a protease and phosphatase inhibitor cocktail (Sigma, St. Louis, Mo.) Samples were sonicated 3 times for 15 see each cycle. Total protein concentrations were determined by the DC Protein Assay (Bio-Rad, Hercules, Calif.), and 50 μg of each sample were run on 4-20% Tris-glycine SDS-PAGE gels. Proteins were transferred to nitrocellulose membranes. and blots were blocked with 5% milk PBS Tween 20. Primary antibody was incubated at 4° C. overnight. Secondary antibodies were goat anti-rabbit 770 and goat anti-mouse 680 (1:10000, Biotium). Fluorescence immunoreactivity was imaged on a LI-COR Odyssey and quantified using Image Studio Lite Software. α-synuclein expression was normalized to β-actin expression in the same lane. The experiment was repeated twice and represents two independent biological replicates.

Mitochondrial superoxide and Cell viability assays. MD NPCs were seeded at 3.5×10⁴ cells/mm² and cultured in high glucose N227 medium without phenol red in black 96-well plates (Greiner). High Throughput Screening plate reader analysis (FLUOstar Omega, BMG) was conducted Briefly, 24 hours after plating, MD NPCs were treated with 20 μM rotenone for 18 h or with DMSO only. The MitoSox assay was used for the detection of mitochondria-associated superoxide levels. Adherent NPCs in 96-well plates were incubated with 2 μM MitoSOX™ (Ex./Em. 510 nm/580 nm) and 2 μM MitoTracker® Green (485 nm/520 nm) (Life Technologies) in high glucose medium without phenol red for 15 min at 37° C. in the dark. Cells were washed twice with medium containing 1 μM Hoechst 33342. Fluorescence was detected by sequential readings, and MitoSOX™ signals were normalized to mitochondrial content (Mitrotracker®) and cell number (Hoechst).

The C12 resazurin assay was used to measure cell viability. Briefly, cells were prepared as above and then loaded with 3 μM C-12 Resazurin (Ex./Em: 563/587 nm) (Life Technologies) in high glucose medium without phenol red for 30 min at 37° C. in the dark. Cells were washed twice with medium containing 1 μM Hoechst 33342. C12-Resazurin fluorescence intensities were normalized to Hoechst fluorescence Each experiment was performed in 6 technical replicates per MD NPCs transduced line, and each experiment was repeated twice and represents two independent biological replicates.

Global DNA methylation. DNA from each stably transduced MD NPC line was extracted using DNeasy Blood and Tissue Kit (Qiagen). Global DNA methylation was assessed using a commercially available 5-methyleytosine (5-mC)-based immunoassay platform (MethylFlash™ Global DNA Methylation (5-mC) ELISA Kit, Epigentek). according to the manufacturer's instructions. Briefly, purified DNA (100 ng) and unmethylated (negative) control DNA (10 ng) were incubated in strip wells with a solution to promote DNA binding and adherence to the well. The samples in the strip-wells were treated with solutions containing the diluted 5-mC capture and the detection antibodies. The methylated fraction of DNA was quantified colorimetrically by absorbance readings using a FLUOstar Optima. BMG. The percentage of methylated DNA was calculated as a proportion of the optical density (OD), according to manufacturers' instructions using the formula;

$$5 mC ( \% ) = \frac{Sample OD - Negative Control OD}{( Slope * ng DNA )} * 100$$

The percentage of 5-mC was determined using two replicates in each of the two independent experiments.

Statistical analysis. The significance of the differences between the MD NPCs stable lines and across the different conditions were analyzed statistically using the following pairwise comparisons tests (GraphPad Prism7): (i) Two-group comparisons using Student's t tests; (ii) Multiple comparisons using Dunnett's method.

### Example 2

### Development of the Novel Lentiviral Vector System for Efficient Delivery of Epigenetic-CRISPR/Cas9 Based Tools

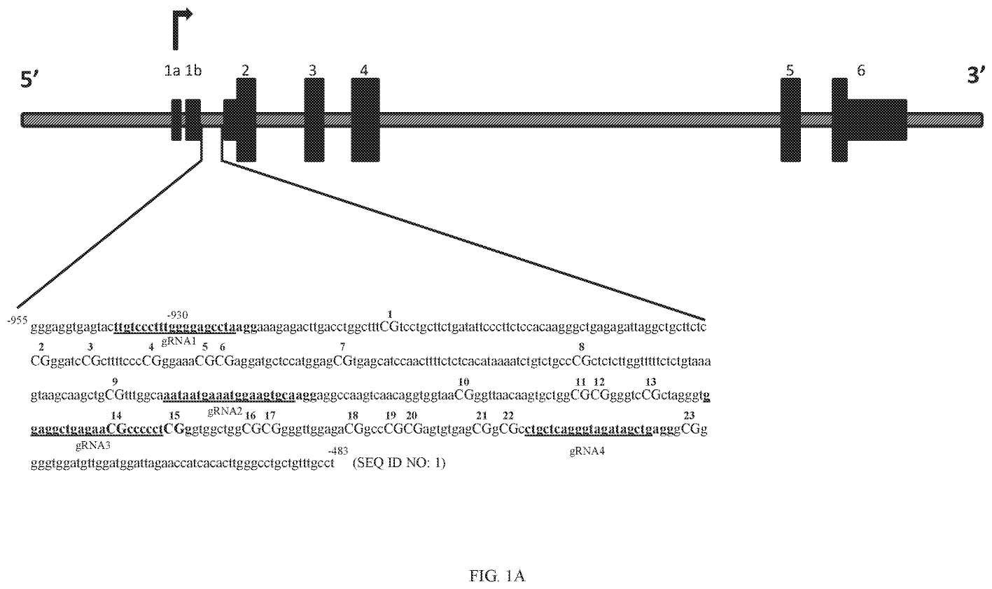

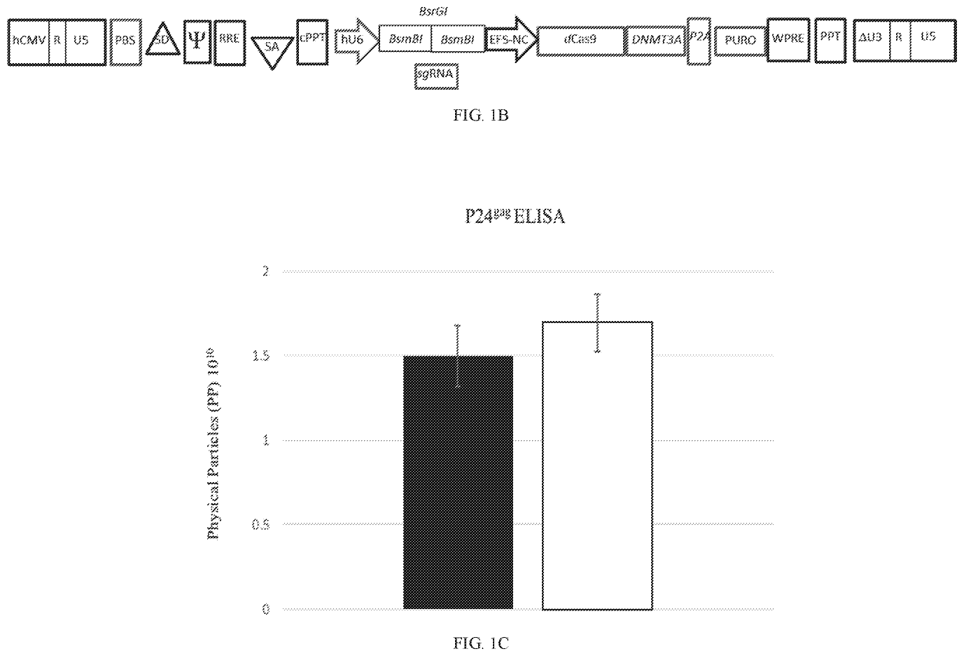

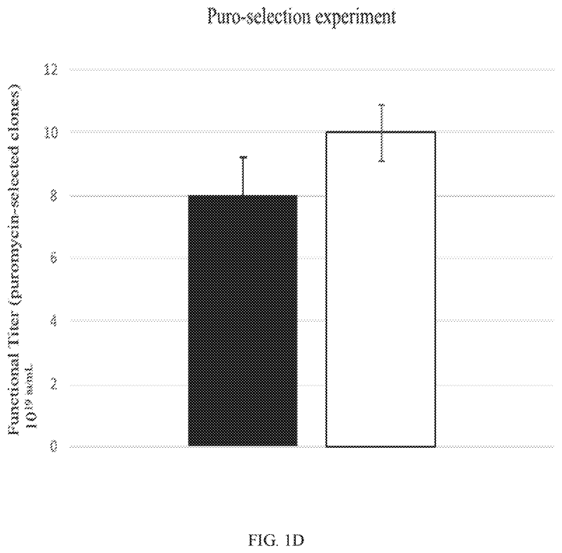

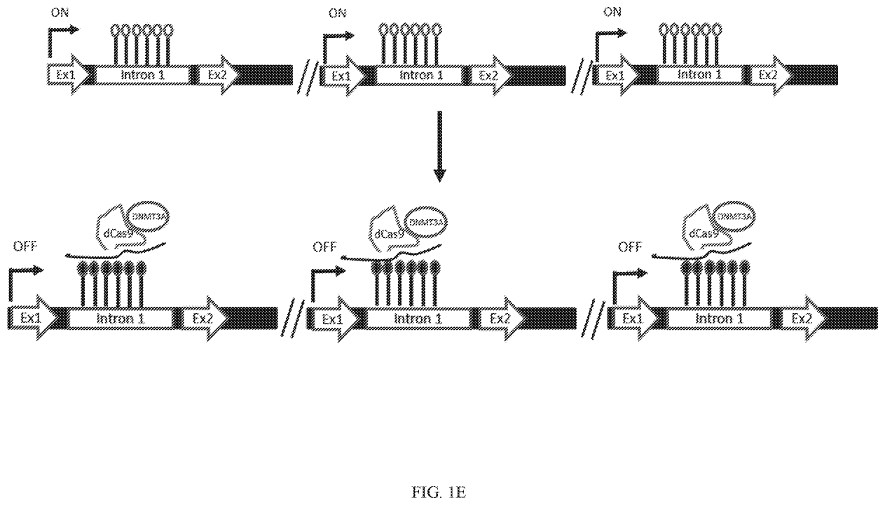

One shortcoming of all-in-one integrating lentiviral vector systems used for the delivery of CRISPR/Cas9-based materials is low production titers. Methods to overcome such problems have included development of binary-plasmid vector systems in which the Cas9 and gRNA components are delivered separately. This approach has improved production yields, but is not suitable for gene-editing applications including in-vivo screening and disease-modeling. The second generation of all-in-one vectors that have been recently developed show increase in production titer and transduction efficiency over the first-generation systems, but these are still about 25-fold lower production yields compared with traditional vectors. The ability to simultaneously deliver Cas9 and sgRNA through a single vector enables facile and robust in vivo gene editing. which is particularly advantageous for developing a translatable gene therapy-products. The present disclosure relates to an effective means of lentiviral vector-mediated CRISPR/Cas9-gene transfer by including in the LV-expression cassette Sp1-transcription factor binding sites (upstream from human U6 (hU6) promoter). and a state-of-art U3′ deletion that eliminates the TATA box from 5′ U3 (FIG. 1B). This novel system can be efficiently packaged into integrase-competent lentiviral particles (ICLV) and integrase-deficient lentiviral particles (IDLV). Furthermore, the system is capable of mediating rapid and robust gene editing in human embryonic kidney (HEK293T) cells and post-mitotic brain neurons in vivo.

To further develop the lentiviral vector system for epigenetic-based gene editing perturbations, the backbone was further modified by integrating into it a dCas9-DNMT3A transgene and creating ICLV-dCas9-DNMT3A-puromycin/GFP and IDLV-dCas9-DNMT3A-puromycin/GFP vectors (for the IDLV vectors a point mutation (D64E) has been introduced into the catalytic domain of the Int gene (FIG. 1B). The production titers of the resulting vectors were measured using a p24gag ELISA assay. The titers for both ICLV-dCas9-DNMT3A and IDLV-dCas9-DNMT3A were found to be at the range of 1-2×10¹⁰ vg/ml, which is comparable with the titers obtained from naïve-lentiviral vector systems (FIG. 1C). We further assessed the production efficiency of the novel ICLV-system using an antibiotic-resistance (puromycin) colony forming assay (FIG. 1D) The ICLV-dCas9-DNMT3A and a naïve ICLV vector (LV-CMV-Puro) vectors demonstrated similar packaging efficiency and expression capability (FIG. 1D).

### Example 3

### Results—Targeted Methylation of SNCA-Intron 1 Using all-in-One Lentiviral Vector-dCas9-DNMT3A System

SNCA intron 1 contains a region of CpG island (CGI) [Chr4: 89,836,150-89,836,593 (GRCh38-hg38)] that comprised of 23 CpGs (FIG. 1A), in which the methylation status altered along with increased SNCA expression. Furthermore, SNCA intron 1 sub-region may be differentially methylated in disease state CpG sites within this sub-region of intron 1 could be candidate targets for epigenetically manipulation, associated with fine regulation of SNCA transcription, whereas enhancement in DNA-methylation in these CpG sites may allow tight downregulation of SNCA expression and reversion of PD related phenotype. To evaluate this premise, an all-in-one gRNA-dCas9-DNMT3A lentiviral vector was constructed using the production- and expression-optimized backbone that contains a repeat of transcription factor Sp1-binding sites upstream from human U6 (hU6) promoter, and a state-of-the-art deletion within the U3′ region of 3′ long terminal repeat (LTR) (FIG. 1B) This backbone vector is highly efficient in delivering and expressing CRISPR/Cas9 components. The backbone has been cloned with a fused version of dCas9-DNMT3A protein expressed downstream from gRNA-cassette (FIG. 1B). Four gRNAs targeting different CpGs within SNCA intron were designed and cloned into the parental vector 1 (FIG. 1A).

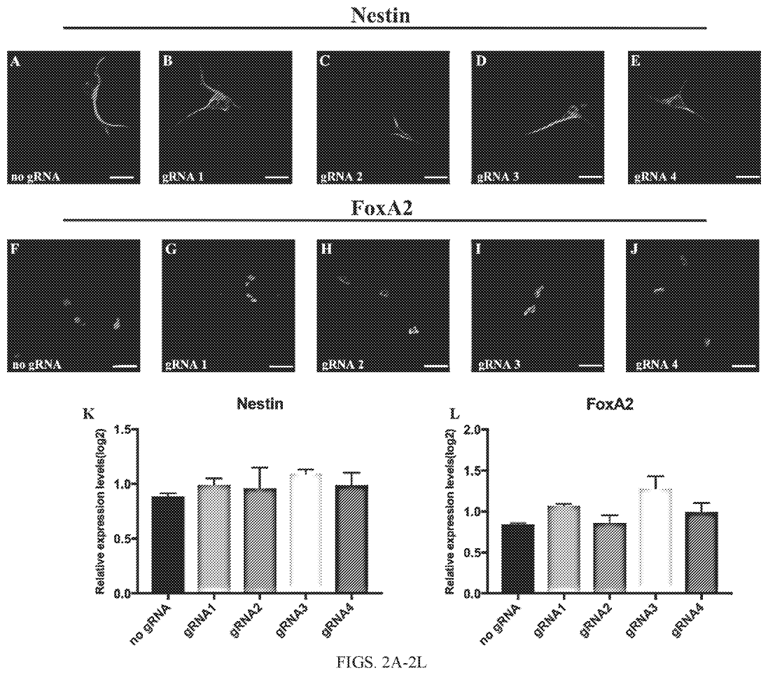

Patients with the triplication of the SNCA locus show a constitutively double expression of the SNCA-mRNA expression levels, and manifest early onset of PD. Therefore, the SNCA-Tri cell lines represent an adequate model to study PD in the context of the overexpression of SNA. To test whether the enhancement in DNA-methylation in the CpG islands within intron 1 will downregulate SNCA gene expression as proposed in FIG. 1C, the gRNA-dCas9-DNMT3A expression cassette was packaged into lentiviral vector and the resulting particles were transduced into hiPSC line derived from a patient with SNCA triplication (SNCA-Tri) that was differentiated into dopaminergic progenitor neurons (MD NPC), the primarily neuronal type affected in PD. To revalidate the neuronal type and differentiation stage, the stably transduced hiPSC-derived MD NPC lines were characterized by immunofluorescent and real-time RT-PCR using Nestin and forkhead box protein A2 (FOXA2), specific markers for MD NPCs (FIG. 2)

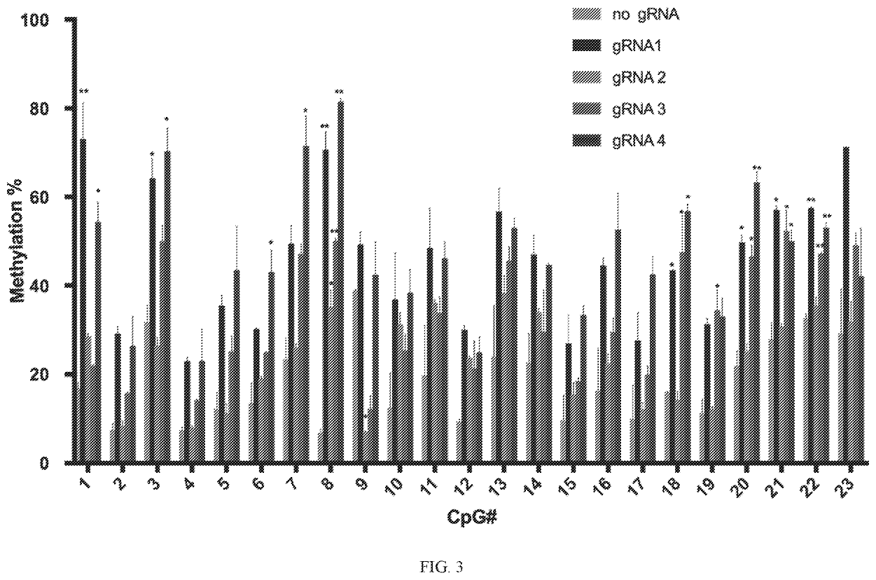

Next, the percentage of the methylation of each of the individual 23 CpGs in SNCA intron 1 was quantitatively determined for each of the five stably transduced hiPSC-derived MD NPC lines. FIG. 3 and Table 5 present the % of methylation at the individual CpG sites for each hiPSC-derived MD NPC line stably carrying a gRNA-dCas9-DNMT3A transgene and indicate the significance of the increase in methylation % relative to the control MD NPC no-gRNA line. Each gRNA-dCas9-DNMT3A transgene led to significant increased methylation of several CpGs across SNCA intron 1 compared to the line carrying the dCas9-DNMT3A no-gRNA transgene. It is worth nothing that while some significantly hypermethylated CpGs were exclusive for a particular MD NPC line (gRNA2 CpG 9, gRNA3 CpG 19: gRNA4 CpG 6 and 7), several CpGs were modified in multiple gRNA transgene cell lines (gRNA 1 and 4>CpG 1, 3, all gRNAs>CpG 8, gRNA 1, 3 and 4>CpG 18, 20-22) (FIG. 3, Table 5).

TABLE 5
% of methylation at the individual 23 CpG sites
in the hiPSC-derived MD NPC lines stably carrying
the different gRNA-dCas9-DNMT3A transgenes

| | | | | | p value |
|---|---|---|---|---|---|
| | | | | p value | (Corrected for 23 |
| | | Average | S.E.M | (Dunnett's) | comparisons) |
| CpG 1 | no gRNA | 16.885 | 0.815 | | |
| | gRNA1 | 73.05 | 5.88 | 0.00002 | 0.00046 |
| | gRNA2 | 28.64 | 0.35 | 0.109 | 2.507 |
| | gRNA3 | 21.915 | 0.175 | 0.6218 | 14.3014 |
| | gRNA4 | 54.37 | 3.18 | 0.001 | 0.023 |
| CpG 2 | no gRNA | 7.53 | 1.01 | | |
| | gRNA1 | 29.14 | 1.11 | 0.0031 | 0.0713 |
| | gRNA2 | 8.355 | 0.785 | 0.996 | 22.908 |
| | gRNA3 | 15.755 | 0.175 | 0.1304 | 2.9992 |
| | gRNA4 | 26.42 | 4.71 | 0.0056 | 0.1288 |
| CpG 3 | no gRNA | 31.815 | 2.635 | | |
| | gRNA1 | 64.13 | 3.19 | 0.0013 | 0.0299 |
| | gRNA2 | 26.515 | 1.265 | 0.5283 | 12.1509 |
| | gRNA3 | 49.97 | 2.57 | 0.0167 | 0.3841 |
| | gRNA4 | 70.3 | 3.65 | 0.0006 | 0.0138 |
| CpG 4 | no gRNA | 7.455 | 0.435 | | |
| | gRNA1 | 22.97 | 0.58 | 0.0144 | 0.3312 |
| | gRNA2 | 8.015 | 0.265 | 0.9991 | 22.9793 |
| | gRNA3 | 14.145 | 0.125 | 0.2403 | 5.5269 |
| | gRNA4 | 23.005 | 5.065 | 0.0143 | 0.3289 |
| CpG 5 | no gRNA | 12.285 | 2.505 | | |
| | gRNA1 | 35.48 | 1.69 | 0.0194 | 0.4462 |
| | gRNA2 | 11.33 | 1.44 | 0.9989 | 22.9747 |
| | gRNA3 | 25.145 | 2.485 | 0.1511 | 3.4753 |
| | gRNA4 | 43.5 | 7.11 | 0.0055 | 0.1265 |
| CpG 6 | no gRNA | 13.54 | 3.17 | | |
| | gRNA1 | 30.225 | 0.115 | 0.0076 | 0.1748 |
| | gRNA2 | 19.1 | 0.3 | 0.3059 | 7.0357 |
| | gRNA3 | 24.905 | 0.095 | 0.0365 | 0.8395 |
| | gRNA4 | 43.005 | 3.515 | 0.0006 | 0.0138 |
| CpG 7 | no gRNA | 23.39 | 3.33 | | |
| | gRNA1 | 49.46 | 2.87 | 0.005 | 0.115 |
| | gRNA2 | 25.95 | 0.74 | 0.9257 | 21.2911 |
| | gRNA3 | 47.115 | 1.565 | 0.0075 | 0.1725 |
| | gRNA4 | 71.48 | 4.78 | 0.0003 | 0.0069 |
| CpG 8 | no gRNA | 6.815 | 0.525 | | |
| | gRNA1 | 70.7 | 2.89 | 0.0001 | 0.0023 |
| | gRNA2 | 35.255 | 2.565 | 0.0003 | 0.0069 |
| | gRNA3 | 50.065 | 0.435 | 0.0001 | 0.0023 |
| | gRNA4 | 81.535 | 0.425 | 0.0001 | 0.0023 |
| CpG 9 | no gRNA | 38.895 | 0.175 | | |
| | gRNA1 | 49.245 | 2.025 | 0.113 | 2.599 |
| | gRNA2 | 7.135 | 0.155 | 0.0012 | 0.0276 |
| | gRNA3 | 12.215 | 2.085 | 0.0027 | 0.0621 |
| | gRNA4 | 42.465 | 5.255 | 0.7606 | 17.4938 |
| CpG 10 | no gRNA | 12.365 | 5.615 | | |
| | gRNA1 | 36.895 | 7.495 | 0.0407 | 0.9361 |
| | gRNA2 | 31.28 | 1.86 | 0.0996 | 2.2908 |
| | gRNA3 | 25.36 | 2.57 | 0.2743 | 6.3089 |
| | gRNA4 | 38.41 | 3.67 | 0.0325 | 0.7475 |
| CpG 11 | no gRNA | 19.835 | 7.875 | | |
| | gRNA1 | 48.495 | 6.315 | 0.0241 | 0.5543 |
| | gRNA2 | 36.13 | 0.53 | 0.164 | 3.772 |
| | gRNA3 | 33.815 | 2.565 | 0.2427 | 5.5821 |
| | gRNA4 | 46.1 | 2.63 | 0.0339 | 0.7797 |
| CpG 12 | no gRNA | 9.435 | 0.245 | | |
| | gRNA1 | 30.015 | 0.685 | 0.0043 | 0.0989 |
| | gRNA2 | 23.705 | 0.215 | 0.0207 | 0.4761 |
| | gRNA3 | 21.265 | 4.425 | 0.0426 | 0.9798 |
| | gRNA4 | 24.935 | 2.525 | 0.0148 | 0.3404 |
| CpG 13 | no gRNA | 24.07 | 8.15 | | |
| | gRNA1 | 56.695 | 3.745 | 0.0095 | 0.2185 |
| | gRNA2 | 38.45 | 2.69 | 0.1774 | 4.0802 |
| | gRNA3 | 45.54 | 2.28 | 0.0501 | 1.1523 |
| | gRNA4 | 53.04 | 1.61 | 0.0157 | 0.3611 |
| CpG 14 | no gRNA | 22.66 | 4.59 | | |
| | gRNA1 | 47.05 | 3.03 | 0.0185 | 0.4255 |
| | gRNA2 | 33.96 | 0.55 | 0.2343 | 5.3889 |
| | gRNA3 | 29.68 | 6.54 | 0.5564 | 12.7972 |
| | gRNA4 | 44.675 | 0.235 | 0.0278 | 0.6394 |
| CpG 15 | no gRNA | 9.615 | 4.025 | | |
| | gRNA1 | 26.95 | 4.56 | 0.0245 | 0.5635 |
| | gRNA2 | 15.465 | 1.855 | 0.4927 | 11.3321 |
| | gRNA3 | 18.48 | 0.48 | 0.2184 | 5.0232 |
| | gRNA4 | 33.455 | 1.405 | 0.0065 | 0.1495 |
| CpG 16 | no gRNA | 16.245 | 6.775 | | |
| | gRNA1 | 44.505 | 1.255 | 0.0143 | 0.3289 |
| | gRNA2 | 22.395 | 1.505 | 0.7005 | 16.1115 |
| | gRNA3 | 29.59 | 2.13 | 0.1909 | 4.3907 |
| | gRNA4 | 52.68 | 5.71 | 0.0048 | 0.1104 |
| CpG 17 | no gRNA | 9.955 | 5.325 | | |
| | gRNA1 | 27.655 | 4.455 | 0.042 | 0.966 |
| | gRNA2 | 12.145 | 1.085 | 0.975 | 22.425 |
| | gRNA3 | 19.89 | 1.35 | 0.245 | 5.635 |
| | gRNA4 | 42.575 | 2.775 | 0.0033 | 0.0759 |
| CpG 18 | no gRNA | 15.97 | 0.11 | | |
| | gRNA1 | 43.49 | 0.15 | 0.0023 | 0.0529 |
| | gRNA2 | 14.16 | 1.33 | 0.9638 | 22.1674 |
| | gRNA3 | 47.63 | 5.71 | 0.0012 | 0.0276 |
| | gRNA4 | 56.825 | 1.105 | 0.0004 | 0.0092 |
| CpG 19 | no gRNA | 11.215 | 2.255 | | |
| | gRNA1 | 31.28 | 0.97 | 0.0042 | 0.0966 |
| | gRNA2 | 12.24 | 0.32 | 0.9906 | 22.7838 |
| | gRNA3 | 34.44 | 3.18 | 0.0022 | 0.0506 |
| | gRNA4 | 33.06 | 2.93 | 0.0029 | 0.0667 |
| CpG 20 | no gRNA | 21.87 | 2.39 | | |
| | gRNA1 | 49.72 | 1.19 | 0.0003 | 0.0069 |
| | gRNA2 | 25.14 | 1.32 | 0.5342 | 12.2866 |
| | gRNA3 | 46.525 | 1.825 | 0.0005 | 0.0115 |
| | gRNA4 | 63.27 | 1.66 | 0.0001 | 0.0023 |
| CpG 21 | no gRNA | 27.865 | 2.565 | | |
| | gRNA1 | 57.1 | 0.6 | 0.0005 | 0.0115 |
| | gRNA2 | 30.8 | 0.36 | 0.7065 | 16.2495 |
| | gRNA3 | 52.39 | 3.19 | 0.001 | 0.023 |
| | gRNA4 | 50.015 | 1.715 | 0.0017 | 0.0391 |
| CpG 22 | no gRNA | 32.68 | 0.68 | | |
| | gRNA1 | 57.5 | 0.13 | 0.0001 | 0.0023 |
| | gRNA2 | 35.665 | 1.245 | 0.0961 | 2.2103 |
| | gRNA3 | 47.225 | 0.265 | 0.0001 | 0.0023 |
| | gRNA4 | 53.07 | 0.78 | 0.0001 | 0.0023 |
| CpG 23 | no gRNA | 29.19 | 7.07 | | |
| | gRNA1 | 71.26 | 0.14 | 0.0054 | 0.1242 |
| | gRNA2 | 31.84 | 3.17 | 0.9837 | 22.6251 |
| | gRNA3 | 49.125 | 1.885 | 0.0976 | 2.2448 |
| | gRNA4 | 42.12 | 7.64 | 0.3064 | 7.0472 |

### Example 4

### Downregulation of SNCA-mRNA and Protein Levels

Previous reports show that changes in intron 1 methylation regulate SNCA transcription. The present example tested whether DNA-methylation editing of SNCA-intron 1 can reduce the endogenous expression level of SNCA-mRNA and α-synuclein protein using the hiPSC-derived MD NPC lines carrying the dCas9-DNMT3A gRNAs.

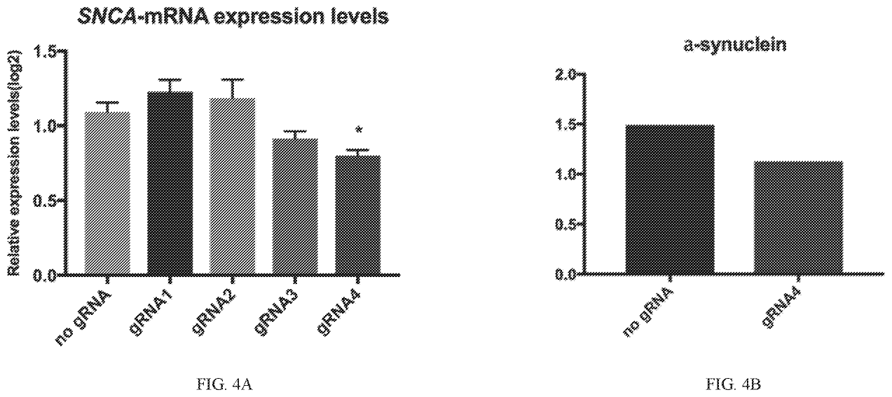

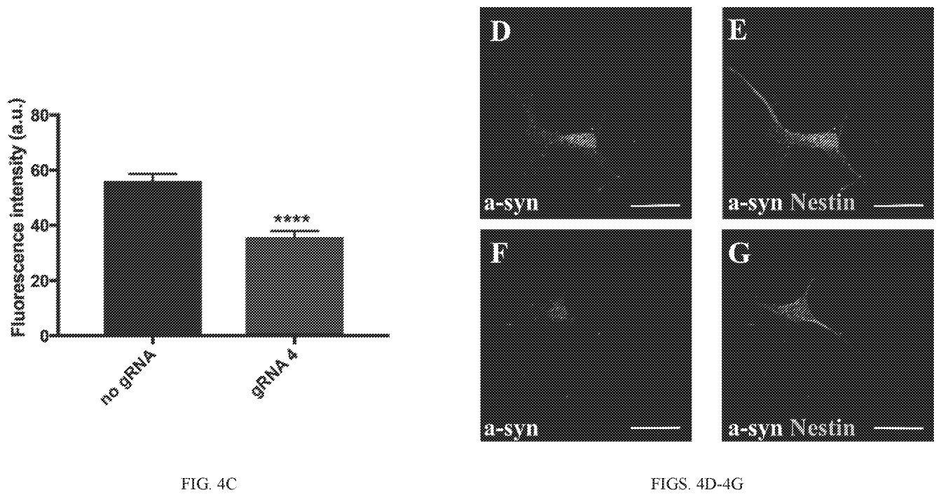

First, the SNCA-mRNA expression levels in hiPSC-derived MD NPC transduced with each of the gRNA-dCas9-DNMT3A vectors was measured. The expression level of SNCA-mRNA in the MD NPC line carrying the gRNA4-dCas9-DNMT3A transgene was significantly lower, amounting to ˜30% reduction (p=0.006; Student's t test), than that observed for the control MD NPC line carrying the dCas9-DNMT3A no-gRNA counterpart (FIG. 4A) The MD NPC with the gRNA3-contained transgene also showed a reduction in SNCA-mRNA levels compared to MD NPC with the no-gRNA transgene, however, this reduction was subtler and didn't reach statistical significance (17% reduction, p=0.06; Student's t test). No significant effects on SNCA-mRNA expression were observed in MD NPC lines with the gRNA1—or the gRNA2-contained transgenes (p=0.2286 and p=0.5248, respectively), indicating that the modified CpGs and/or the extent of the change in methylation rate were not sufficient to drive alteration in transcript expression in these lines. The integrated results of the DNA-methylation profiles with the changes in SNCA-mRNA expression for all MD NPC lines provide clues for the CpGs sites within SNCA intron 1 that are associated with transcriptional regulation of SNCA gene. Accordingly, CpG sites 6, 7 are strong candidate targets for methylation manipulation towards normalizing SNCA expression levels.

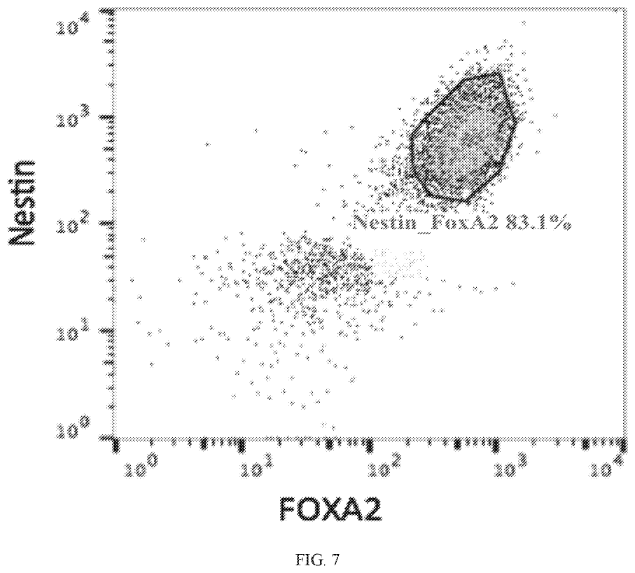

Next, the effect of the system on α-synuclein protein expression levels in the MD NPC line stably transduced with the gRNA4-dCas9-DNMT3A vector was evaluated. In accordance with the SNCA-mRNA results, the endogenous α-synuclein protein abundance was decreased by nearly 25%, compared with those in the control MD NPC line that carried the no-gRNA transgene (p=0.0055: Student's t test) (FIG. 4B). α-synuclein levels in the ‘pure’ population of MD NPCs were further validated by immunofluorescent using double staining for SNCA and the MD NPC marker, Nestin. Analysis of the double stained cells confirmed the reduction in the endogenous α-synuclein levels, amounting to ˜36% lower levels in the gRNA4MD NPC line vs the control no-gRNA line (p<0.0001; Student's t test) (FIG. 4C-G) Of note, the successful differentiation rate of MD NPC is ˜80%, this may explain the greater effect on α-synuclein levels observed by double immunofluorescent approach as it constrained the analysis to the differentiated neurons only vs western blot and real-time PCR analyses that comprised of the whole cell culture (FIG. 7).

Collectively, these consistent data suggest that hypermethylation of intron 1 conferred by the dCas9-DNMT3A transgene that contained gRNA4 was sufficient for altering endogenous SNCA-mRNA expression and α-synuclein protein levels significantly (p: 0.006 and 0.0055, respectively), resulting in an increase in methylation levels and relative lower SNCA-mRNA and protein abundance, compared the control cell carrying the no-gRNA transgene (FIG. 4).

### Example 5

### Rescue of SNCA-Tri Cellular Phenotypes

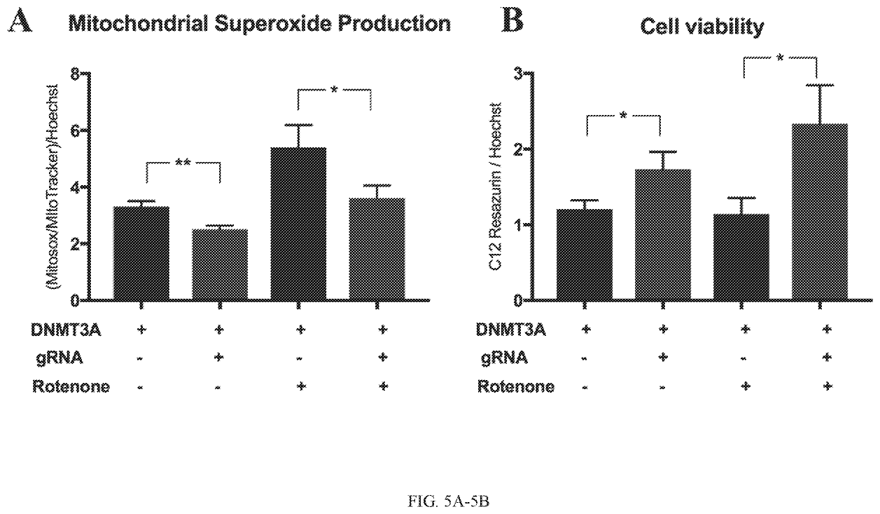

PD is characterized by loss of neurons in the substantia nigra and elsewhere. and overexpression of SNCA in neuronal cell culture inducing apoptotic cell death. In addition, mitochondria dysfunction, measured by higher mitochondrial reactive oxygen species (ROS) production, has been associated with PD. In accordance, the SNCA-Tri hiPSC-derived neurons show reduced viability and increased mitochondria associated superoxide production wider exposure to the environmental mitochondrial complex I toxin rotenone. The effect of the reduction in α-synuclein levels mediated by intron 1 hypermethylation on the cellular phenotypes characteristic of the SNCA-Tri hiPSC-derived NPC, i.e. mitochondrial superoxide production and cell viability, was determined by comparing the MD NPC line carrying the gRNA4-contained transgene to the control no-gRNA transgene. MD NPCs expressing the cassette that contains gRNA4 ameliorated the increased mitochondria-associated superoxide production (2.5 vs 3.3, p=0.0016; Student's t test) (FIG. 5A) and demonstrated increased cellular viability (1.7 vs 1.2, p=0.0492; Student's t test) (FIG. 5B) Similarly, under exposure to rotenone (20 μM, 18 hrs) the mitochondria-associated superoxide production was significantly lower (3.6 vs 5.4, p=0.0462; Student's t test) (FIG. 5A) and the viability was significantly higher (2.3 vs 1.1, p=0.0365; Student's t test) (FIG. 5B) in the MD NPCs transduced with the gRNA4-Cas9-DNMT3A vector in comparison to the control no-gRNA counterpart. Overall the effects of the α-synuclein reduction on mitochondria-associated superoxide production and cellular viability, in the cell line expressing the gRNA4, were more pronounced when the cells were challenged with rotenone (25% less superoxide production vs 33% upon rotenone exposure and 1.4-fold increase in viability vs 2-fold with rotenone). These results indicated that the MD NPC line with the gRNA4 is more resistant to stress conditions compared to no-gRNA control cells. Moreover, the gRNA4 MD NPC line exhibited less vulnerability to rotenone compared to the effect of rotenone on the control MD NPC carrying the no-gRNA vector, as measured by 44% vs 63% increase in mitochondria-associated superoxide production, respectively (FIG. 5) Collectively, the results demonstrated that the hypermethylation mediated reduction in SNCA-mRNA accompanied by lower α-synuclein protein levels, rescued the phenotypic perturbations of the SNCA-Tri hiPSC-derived neurons.

### Example 6

### Minimal Effect of gRNA4-dCas9-DNMT3A Transgene on Global Methylation

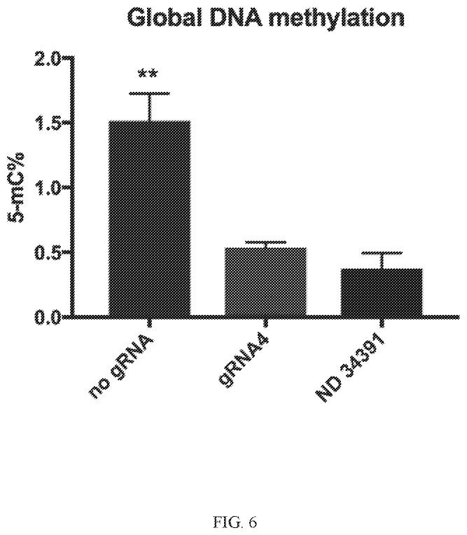

The above examples demonstrate the ability of the gRNA4-dCas9-DNMT3A transgene to mediate robust and sustained methylation across SNCA intron 1 that is sufficient to reverse disease related cellular phenotypes. The target-specificity of the system was next evaluated. To this end, ELISA-based immunoassay was employed to quantify the global DNA-methylation by measuring the percentage of the 5-methylcitosine (5-mC %) (40) of the stably transduced hiPSC-derived MD NCP samples that carry gRNA4 and no-gRNA compared to the untransduced SNCA-Tri MD NPC line (FIG. 6). The hiPSC-derived MD NPC line that constitutively expresses the gRNA4-dCas9-DNMT3A transgene showed no significant change in 5-mC %. compared to the original SNCA-Tri MD NPC line. 0.53% vs. 0.37%, respectively (p=0.97) (FIG. 6). On the other hand, the SNCA-Tri/no-gRNA dCas9-DNMT3A line demonstrated a significant increase in global DNA-methylation (5-mC % 0.37% vs 1.51%, p=0.009) (FIG. 6). The steady global DNA-methylation observed in the cell line carrying the gRNA4-dCas9-DNMT3A transgene suggests that the off-target of the DNA methylation is minimal. Thus, supporting the validity and safety of the system to specifically target the methylation of the CpG island region in SNCA intron 1. In contrast, the transgene that does not contain a gRNA does not sustain a target-specific modification of the DNA-methylation and resulted in increased global methylation.

### Example 7

### Discussion

The human induced Pluripotent Stem Cells (hiPSC)-derived neuron system is a powerful tool to model more accurately aspects of human neurodegenerative diseases including PD It represents a valuable in-vitro system for better understanding the molecular mechanisms underlying neurological diseases and for defining cellular disease processes, and also for efficient drug screening. The advent of hiPSCs derived from PD patients with a genomic triplication of the SNCA gene (SNCA-Tri) provides a unique and valuable tool for the development of novel therapeutic avenues that target SNCA expression levels. Herein, this model system is used to evaluate epigenome editing as a strategy, for tight downregulation of SNCA back to normal physiological levels required to maintain neuronal function.

Herein, all-in-one lentiviral vectors expressing four gRNAs targeting different regions of the CpG islands in SNCA intron 1 were used. The transduction of each of the gRNA-vectors resulted in the enhancement of DNA methylation of multiple CpGs within SNCA intron 1. However, only one gRNA, gRNA4, positioned at the 3′ of the CpG island region resulted in repression of SNCA-mRNA levels. Noteworthy, each gRNA vector resulted in a specific modification of the DNA-methylation profile across the human SNCA intron 1. Substantial changes of specific CpG sites within the 23 sites may influence transcription efficiency more effectively than others. Therefore, hypermethylation of these particular CpG sites may be involved for turning the methylation editing into transcriptional deactivation. Based on the combined results presented herein, CpG sites 6 and 7 may be strong targets for pharmaceutical methylation editing to exert tight regulation for achieving normalized SNCA expression levels.

Accurate and efficient targeting is the ultimate goal for gene therapy in PD caused by SNCA dysregulation, and epigenome editing is an attractive strategy toward therapeutic intervention. The outcomes of this work address a critical obstacle essential in the development of therapeutic drugs, as it's important to develop new strategies to reduce SNCA overexpression in a controlled manner.

### Example 8

### Downregulation of SNCA Expression in Rat Cell Line

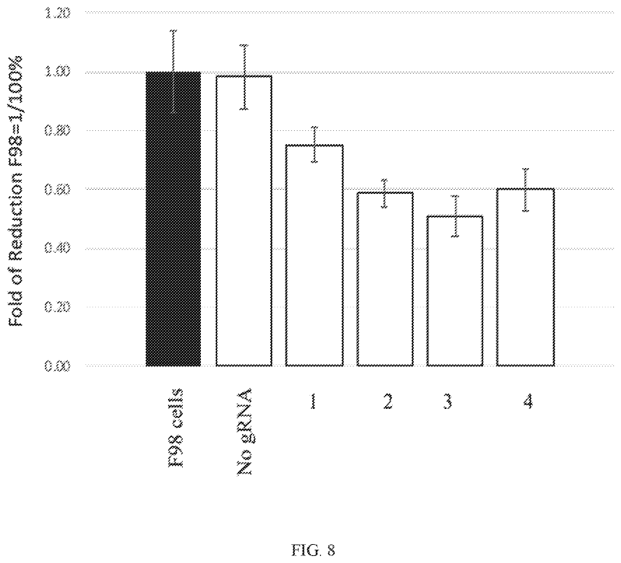

SNCA-mRNA in rat F98 cell line were transduced with lentiviral vector harboring gRNA-dCas9-DNMT3A transgenes. Levels of SNCA-mRNA were assessed using quantitative real-time RT-PCR 14 days post-transduction. FIG. 8 shows the levels of SNCA-mRNA in the different lines (four different gRNA were designed and used, bars 1-4) that were measured by Cyber green-based gene expression assay and calculated relatively to the geometric mean of GAPDHmRNA and PPIA-mRNA reference controls using the 2^{-ΔΔCT} method. Each bar represents the mean of three biological replicates. The results are presented as a fold of reduction from to the naïve (untrasduced) F98 cells (lane 1; black bar). Lane 2: gRNA1: Lane 3: gRNA 2; Lane 4: gRNA3 (pBK744); Lane 5: gRNA 4; Lane 6: gRNA 5. No gRNA control is used in the experiment (pBK539). The error bars represent as the S. D.

### Example 9

### Use of IDLV

Episomal integrase-deficient lentiviral vectors (IDLVs) are an ideal platform for delivery of large genetic cargos where only transient expression of the transgene is desired IDLVs retain residual (integrase-independent and illegitimate) integration rates of ˜0.2%-0.5% (one integration event per 200-500 transduced cells), which could be further reduced by packaging a novel 3′ polypurine tract (PPT)-deleted lentiviral vector into integrase-deficient particles. IDLVs have garnered significant interest among researchers for precise in vivo analysis of genetic diseases, since they significantly reduce the risk of insertional mutagenesis inherent in integrating delivery platforms. The ability to simultaneously deliver Cas9 and sgRNA through a single vector enables facile and robust in vivo gene editing, which is particularly advantageous for developing translatable gene therapy products. Nevertheless, many viral vector platforms, especially those intended for clinical applications are not fully suitable for carrying oversized CRISPR-Cas9 systems. In addition, the production and expression efficiency of these vectors are low. To address these shortcoming, an all-in-one IDLV-CRISPR/Cas9 system for highly efficient gene editing in vitro and in vivo was developed. These vectors permit efficient, rapid, and sustainable CRISPR/Cas9-mediated gene editing in HEK293T cells and post-mitotic brain neurons in vivo. Furthermore, the IDLV-CRISPR/Cas9 system is expressed transiently and has a significantly lower capacity to induce off-target mutations than its integrating counterparts. Taken together, IDLVs are a robust, effective, and safe means for in vivo delivery of programmable nucleases, with substantial advantages over other delivery platforms.

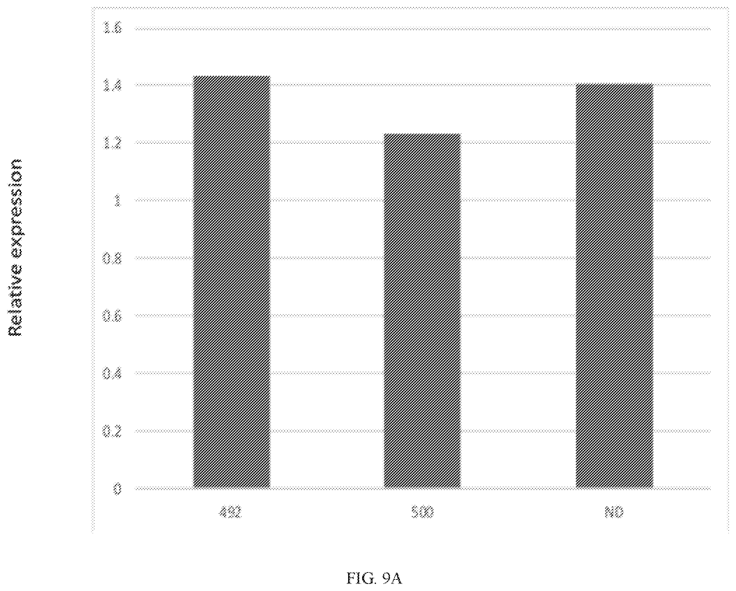

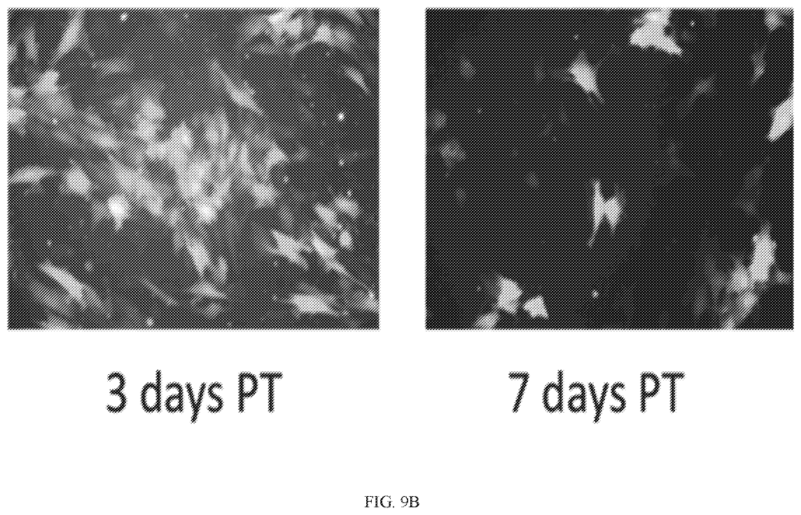

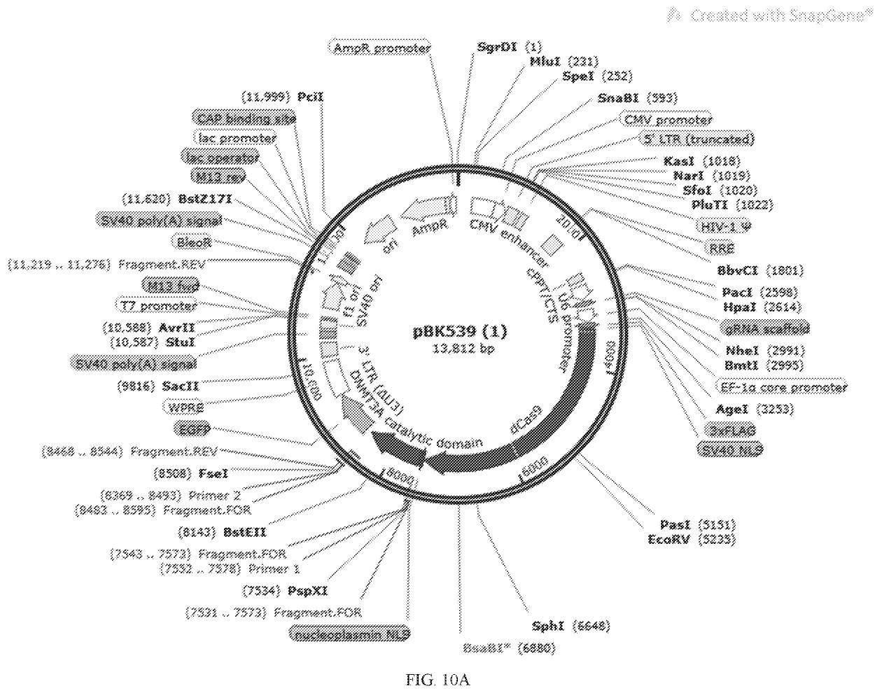

Here, the vector expression cassette was further modified to establish a novel epigenetic editing mean. The novel IDLV vector harbored all-in-one gRNA/CRISPR/dCas9-DNMT3A transgene for efficient and specific targeting DNA methylation within hypomethylated CpG island in the SNCA intron 1 region of neural progenitor cells (NPCs) derived from human induced pluripotent stein cells (hiPSCs) harbored a triplication of the SNCA loci. Levels of SNCA-mRNA were assessed using quantitative real-time RT-PCR 7 days post-transduction. The levels of SNCA-mRNA in the different lines were measured by TaqMan based gene expression assay and calculated relatively to the geometric mean of GAPDH-mRNA and PPIA-mRNA reference controls using the 2^{-DDCT} method (FIG. 9A). In FIG. 9A, each bar represents the mean of four biological and to technical replicates (n=8) for a particular MD NPC line. Lane 1 shows 492 with no gRNA control vector; lane 2 shows 500-gRNA-dCas9-DNMT3A vector and lane 3 shows naïve (untransduced NDs). The error bars represent the S.E.M. We demonstrate that IDLV-gRNA/CRISPR/dCas9-DNMT3A system, similarly to ICLV-gRNA/CRISPR/dCas9-DNMT3A, displayed close to 20% reduction in the SNCA gene expression by 7 days pt (FIG. 9A). Importantly, we show close to 90% reduction in IDLV genomes by day 7 post-transduction (FIG. 9B). These results clearly demonstrate that gRNA/CRISPR/dCas9-DNMT3A delivered by IDLVs is capable of mediating rapid, and sustained reversion of gene activation, and such may be a valid therapeutic strategy for disorders that involve expression dysfunction.

### Example 10

### Rescue of Aging Phenotypes

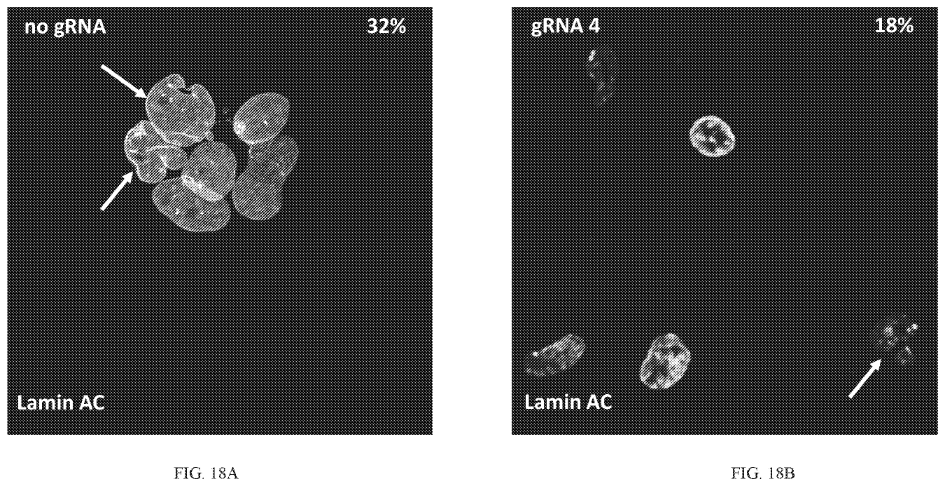

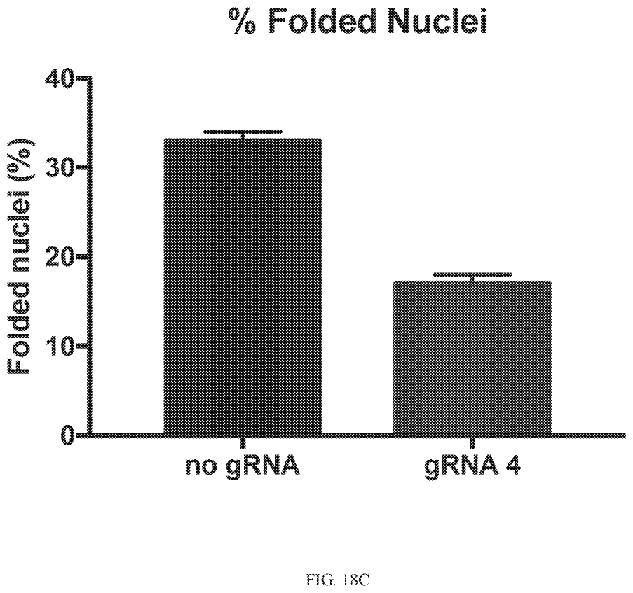

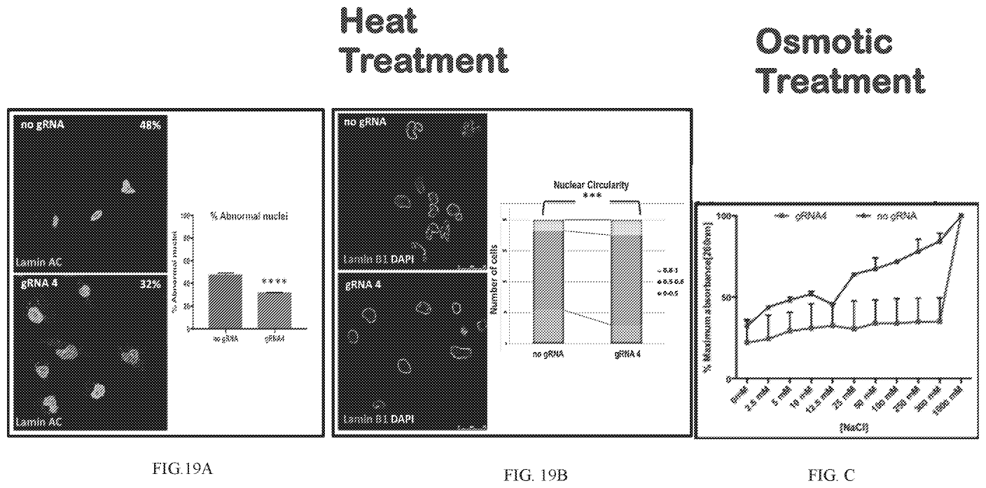

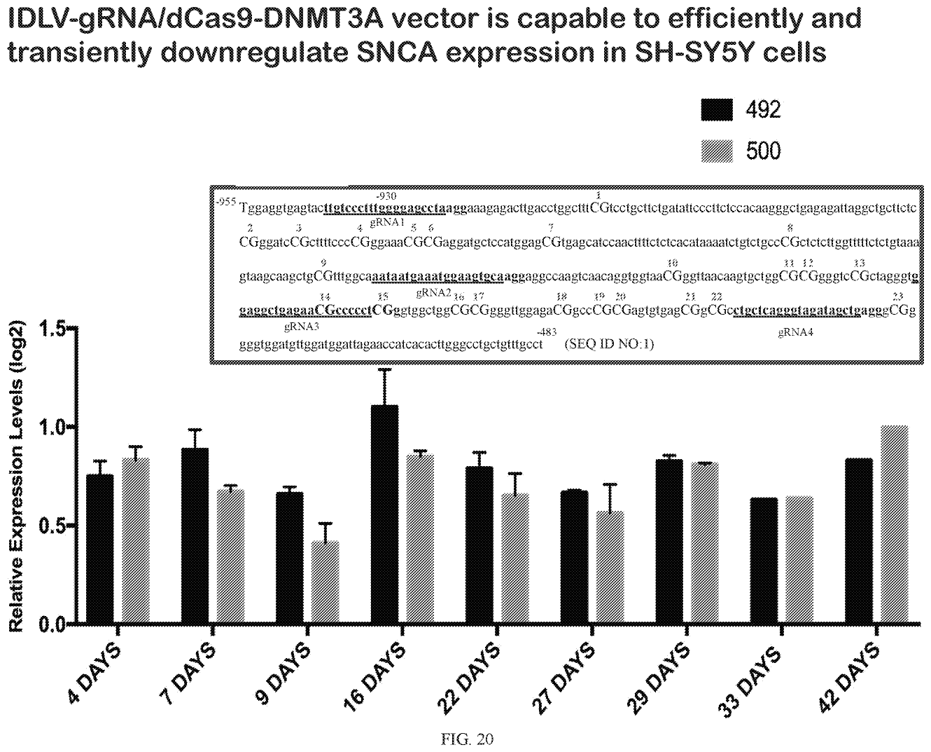

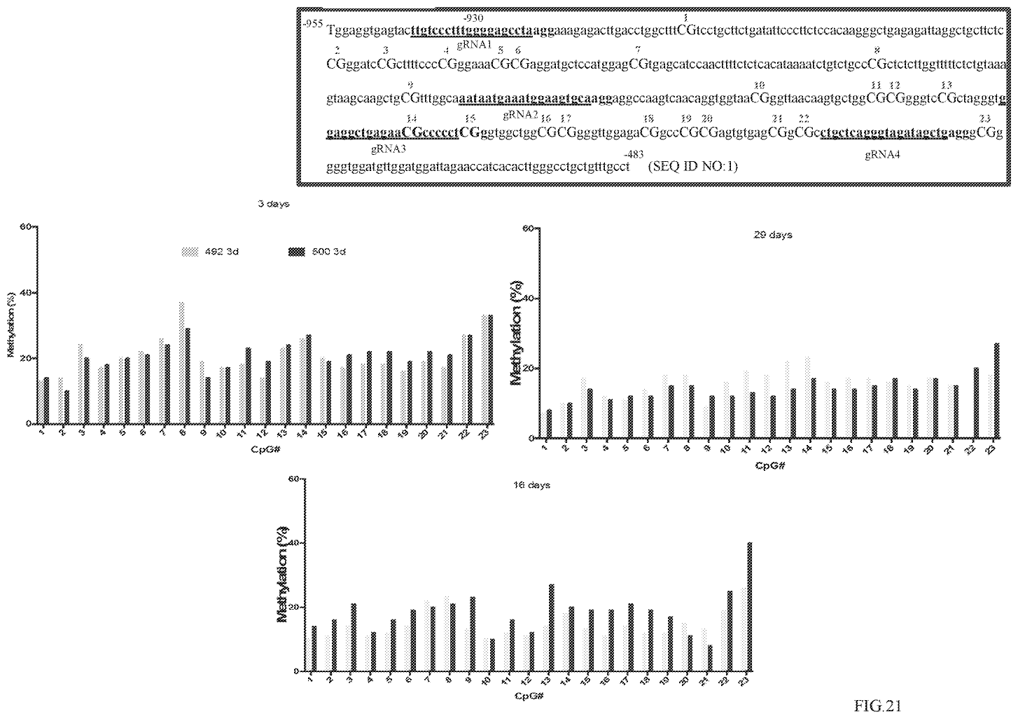

Nuclear folding was analyzed by immunocytochemistry, as described below, using the Lamin A/C marker (Lamin A/C antibody: Ab108595, Abcam), and folded nuclear envelope shape was considered as abnormal. >100 cells per staining were analyzed for two independent experiments (see FIGS. 18A-18C).

Immunocytochemistry: Prior to immunostaining, cells were plated onto PLO/Laminin Coated Cells Imaging Coverglasses (Eppendorf, 0030742060). Cells were fixed in 4% paraformaldehyde and permeabilized in 0.1% Triton-X100. Immunocytochemistry was performed as follow: cells were blocked in 5% goat serum for 1 hour before incubating with primary antibodies overnight at 4° C. Secondary antibodies (Alexa fluor, Life Technologies) were incubated for 1 hour at room temperature. Nuclei were stained with NucBlue® Fixed Cell ReadyProbes® Reagent (ThermoFisher), according to the manufacturers' instructions. Images were acquired on the Leica SP5 confocal microscope using a 40× objective.

The disclosed examples demonstrate the effect of SNCA upregulation (increased expression) on multiple aging-related markers. In general, SNCA multiplication exacerbated neuronal nuclear aging and showed aging signatures already in juvenile stage.

Lamins are involved in the structural integrity of the nuclear envelope and loss of the integrity of the nuclear envelope has been associated with aging. Nuclear envelope integrity was assessed by using the marker Lamin A/C⁹, whereas folded nuclei were counted as abnormal. hiPSC-derived BFCN and mDA derived from a healthy subject showed 13.5% and 14.5% abnormal nuclei, while 2-fold increase in SNCA expression detected in neurons derived from a patient with SNCA triplication (SNCA-Tri) led to significantly higher levels of folded (abnormal) nuclei 56% and 45%, respectively. Thus, overexpression of SNCA resulted in significant increase in nuclei folding, indicating exacerbation of aging signature.

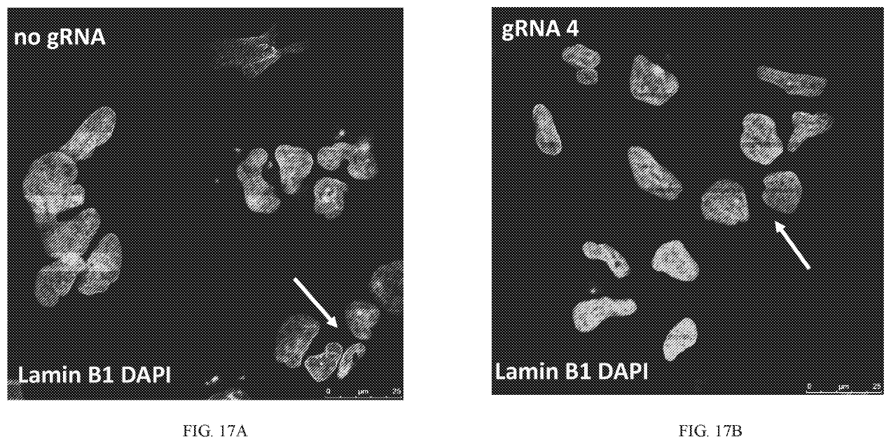

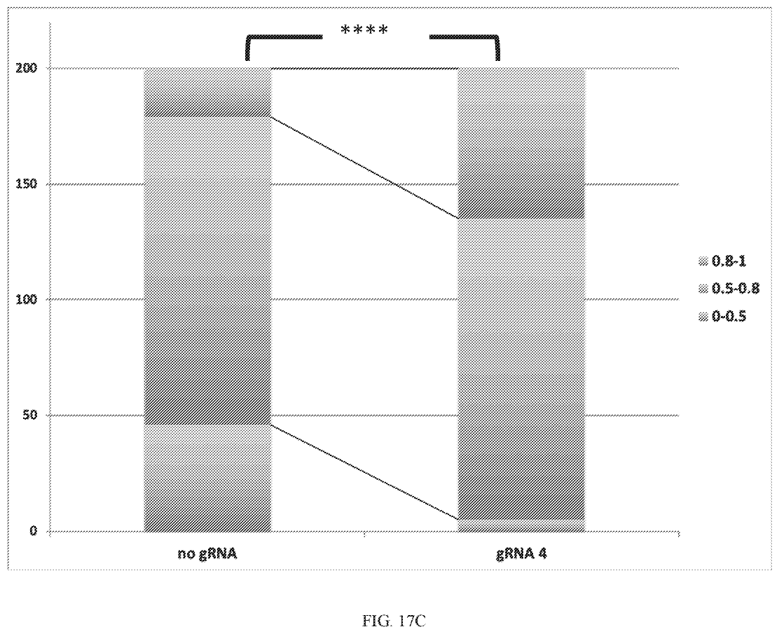

The effect of the reduction in α-synuclein levels mediated by intron 1 hypermethylation on the cellular phenotypes characteristics of the SNCA-Tri hiPSC-derived NPC that are characteristic of aging, i.e. nuclei folding/nuclear circularity, was determined by comparing the MD NPC line carrying the gRNA4-contained transgene to the control no-gRNA transgene. MD NPCs expressing the cassette that contains gRNA4 reversed the increased in abnormal nuclei, demonstrating the rescue of the aging related phenotypes (FIGS. 17-18).

These results extended on the effect of hypermethylation mediated reduction in SNCA-mRNA accompanied by lower α-synuclein protein levels, to the reversion of phenotypic perturbations related to aging.

### Example 11

### Use of CRISPR-Based Epigenome Modifier Based System

To further the understanding of the genetic etiologies and molecular mechanisms that are commonly perturbed in synucleinopathies, and those that may underlie the heterogeneity amongst the different diseases in this group, it is important to characterize in depth isogenic hiPSC-derived models of different pathology-relevant neurons derived from patients and healthy subjects in the context of aging. hiPSCs reprogrammed from fibroblasts obtained from old donors are characterized by molecular and cellular features such as, telomere size, oxidative damage, mitochondrial metabolism, transcriptomic and epigenetic signatures, that are more similar to embryonic stem cells Thus, there is a concern that hiPSC-derived models are not fully suitable for the study of age related conditions.

To address these issues, an optimized and alternative new approach to induce aging in hiPSS-derived neurons was developed. Human induced pluripotent stem cells (hiPSCs) from an apparently healthy individual and a patient with a triplication of the SNCA gene (SNCA670) were purchased from Coriell cell repositories and from the NINDS Human Cell and Data Repository, respectively. GM23280 and ND34391 lines have a normal karyotype. hiPSCs were cultured under feeder-independent conditions in mTeSR™1 medium onto hESC-qualified Matrigel coated plates. Cells were passaged using Gentle Cell Dissociation Reagent (StemCell Technologies) according to the manufacturer's manual. The dopaminergic neurons (mDA) derive from the Ventral Midbrain (MD), while the Basal Forebrain Cholinergic Neurons (BFCN) derive from the Medial Ganglionic Eminence (MGE). Specific protocols were used to differentiate hiPSCs to mDA and BFCN. Differentiation into mDA was performed using an embryoid body-based protocol. hiPSCs were dissociated with Accutase (StemCell Technologies) and seeded into Aggrewell 800 plates (10,000 cells per microwell; Stem Cell Technologies) in Neural Induction Medium (NIM—Stem Cell Technologies) supplemented with Y27632 (10 μM) to form Embryoid Bodies (EBs). On day 5, EBs were replated onto matrigel-coated plates in NIM On day 6, NIM was supplemented with 200 ng/mL SHH (Peprotech) leading to the formation of neural rosettes. On day 12, neural rosettes were selected with Neural Rosette Selection reagent (used per the manufacturer's instructions, StemCell Technologies) and replated in matrigel-coated plates in N2B27 medium supplemented with 3 μM CHIR99021, 2 μM SB431542, 5 μg/ml BSA, 20 ng/ml bFGF, and 20 ng/690 ml EGF, leading to the formation of Neural Precursor Cells (NPCs). Differentiation of NPCs into mDA was initiated 1 day after passaging the NPCs on poly-L-ornithine/laminin-coated plates. NPC maintenance medium was substituted by final differentiation medium consisting of N2B27 medium supplemented with 100 ng/ml FGF8(Peprotech), 2 μM Purmorphamine, 300 ng/ml Dibutyryl cAMP (db-cAMP), and 200 μM L695 ascorbic acid (L-AA) for 14 days. From days 14, cells were fed with maturation medium consisting of 20 ng/ml GDNF, 20 ng/ml BDNF, 10 μM DAPT, 0.5 mM db-cAMP, and 200 μM L-AA. Medium was changed every other day. The differentiation into BFCN was performed as follows. EBs were formed into Aggrewell 800 plates in NIM. On day 5, EBs were replated and the medium was changed daily. From day 8, neural rosettes were grown into NEM (7 parts KO-DMEM to 3 parts F12, 2 mM Glutamax, 1% penicillin and streptomycin, supplemented with 2% B27 (all Life Technologies), plus 20 ng/ml FGF, 20 ng/ml EGF, 5 g/ml heparin, 20 M SB431542 and 10 M Y27632, 1.5M Purmorphamine. On day 12, neural rosettes were selected with Neural Rosette Selection Reagent and replated in NEM onto Matrigel-coated plates. On day 23, Y27632 was withdrawn and final differentiation was performed onto PLO-laminin coated plates in the presence of BrainPhys Medium (Stemcell Technologies) supplemented with N2, B27, BDNF, GDNF, L-ascorbic acid, and db-cAMP until day 45-50. Medium was changed every other day.

To generate juvenile and aged neurons, NPCs were passaged every two days in their respective medium. NPCs were passaged with Accutase (StemCell Technologies) and plated on Matrigel coated plates (2.5*10⁴ cells/cm²). To generate the Juvenile neurons, final differentiation procedures were applied to the NPCs at passages P2-P5 following the protocol outlined above. For the generation of the Aged neurons, NPCs underwent multiple passaging and at passages P14-P16 were differentiated to final neurons.

The above described aged neurons will be used in experiments involving the disclosed compositions. For example, the above described aged neurons may be used with the disclosed compositions in methods for reducing expression of SNCA. For example, the above described IDLV comprising the disclosed composition for epigenome modification of a SNCA gene may be added to the above described aged neurons. Levels of SNCA, α-synuclein, and other markers of aging may be measured in accordance with the methods described herein.

RNA extraction and expression analysis to determine levels of SNCA-mRNA: Total RNA was extracted from each stably transduced MD NPC line using TRIzol reagent (Invitrogen) followed by purification with an RNeasy kit (Qiagen) used per the manufacturer's protocol. RNA concentration was determined spectrophotometrically at 260 nm, while the quality of the purification was determined by 260 nm/280 nm ratio that showed values between 1.9 and 2.1, indicating high RNA quality. cDNA was synthesized using MultiScribe RT enzyme (Applied Biosystems) using the following conditions: 10 min at 25° C. and 120 min at 37° C. Real-time PCR was used to quantify the levels of the MD NPC markers and SNCA expression levels. Briefly, duplicates of each sample were assayed by relative quantitative real-time PCR using TaqMan expression assays and the ABI QuantStudio 7. The particular assays are: Hs00240906 for SNCA target and Hs99999905 and Hs99999904 for the house keeping references, GAPDH and PPIA, respectively.

Each cDNA (20 ng) was amplified in duplicate in at least two independent runs for two independent experiments (overall≥8 repeats), using TaqMan Universal PCR master mix reagent (Applied Biosystems) and the following conditions: 2 min at 50° C., 10 min at 95′C, 40 cycles. 15 sec at 95 (C, and n mm at 60° C. As a negative control for the specificity of the amplification, we used RNA control samples that were not converted to cDNA (no-RT) and no-cDNA/RNA samples (no-template) in each plate. No amplification product was detected in control reactions. Data were analyzed with a threshold set in the linear range of amplification. The cycle number at which any particular sample crossed that threshold (Ct) was then used to determine fold difference, whereas the geometric mean of the two control genes served as a reference for normalization. Fold difference was calculated as 2^{-ΔΔCt}; ΔCt=[Ct(target)−Ct (geometric mean of reference)]. ΔΔCt=[ΔCt(sample)]−[ΔCt(calibrator)]. The calibrator was a particular RNA sample, obtained from the control MD NPCs, used repeatedly in each plate for normalization within and across runs. The variation of the ΔCt values among the calibrator replicates was smaller than 10%.

Western blotting to determine levels of α-synuclein protein: Expression levels of human α-synuclein protein in the stably transduced MD NPC lines were determined by Western blotting with the α-synuclein rabbit monoclonal antibody (ab138501, Abeam; 1:1000) and with mAb β-actin (AM4302, Ambion; 1:5000) for normalization. Cell were scraped from the dish and homogenized in 10× volume of 50 mM Tris-HCl, pH 7.5, 0.150 mM NaCl, 1% Nonidet P-40, in the presence of a protease and phosphatase inhibitor cocktail (Sigma. St. Louis, Mo.). Samples were sonicated 3 times for 15 sec each cycle. Total protein concentrations were determined by the DC Protein Assay (Bio-Rad, Hercules, Calif.), and 25 μg of each sample were run on 12% Tris-glycine SDS-PAGE gels. Proteins were transferred to nitrocellulose membranes, and blots were blocked with 5% milk PBS Tween 20. Primary antibodies were incubated at 4° C. overnight (Abcam, ab138501, 1:1000; Thermofisher AM4302, 1:5000). Horseradish Peroxidase-conjugated secondary antibodies were incubated for 1 h at room temperature (Abcam; 1:10000). Signal was detected with HyGLO Quick Spray (Denville Scientific) and immunoblot were imaged using ChemiDoc MP Imaging System (Biorad). The densitometry was measured using ImageJ software, and α-synuclein expression was normalized to β-actin expression in the same lane. The experiment was repeated twice and represents two independent biological replicates.

Immunocytochemistry quantification of α-synuclein aggregates: Immunofluorescent images of α-synuclein aggregates were analyzed using Leica Application Suite X software. Aggregates number and size were analyzed for 50 cells per cell-line. The baseline for number of aggregates per cells included in the analysis was determined in reference to the number of aggregates observed in the Control cell lines. Size of aggregates was defined in 3 groups: small (<1 μm), medium (1-2 μm), and large (2-5 μm). Frequency distribution plots represent aggregates number and size binned by arbitrary unit increments based on the natural groupings of the data.

Comet assay: Comet assay was used to measure DNA damage in hiPSC-derived neurons applying a protocol as follows. Briefly, mature neurons were lysed in alkaline conditions by placing the slides in A 1 solution [1.2M NaCl, 100 mM Na₂EDTA, 0.1% sodium lauryl sarcosinate, 0.26M NaOH (pH>13)] at 4° C. in the dark for 18-20 hr. Slides were washed three times using A2 solution [(0.03M NaOH, 2 mM Na-EDTA (pH 12.3)], and electrophoresis was conducted for 25 min at a voltage of 0.6V/cm in fresh A2 solution Slides were then washed twice in distilled H₂O for 5 min., subsequently immerged in 70% ethanol, dried for 15 min at room temperature and stained with SYBR Green for 30 min After washing the excess of staining, cells were imaged using a Zeiss Axio Observer Widefield Fluorescence Microscope. Comets were analyzed using the OpenComet Software to determine the Olive Tail Moment, the parameter selected as the quantitative measure for each comet. The OTM was determined in 100 cells, 50 cells per each of two independent comet experiments.

### Example 12

### Validation of Epigenome-Editing Approach In Vivo

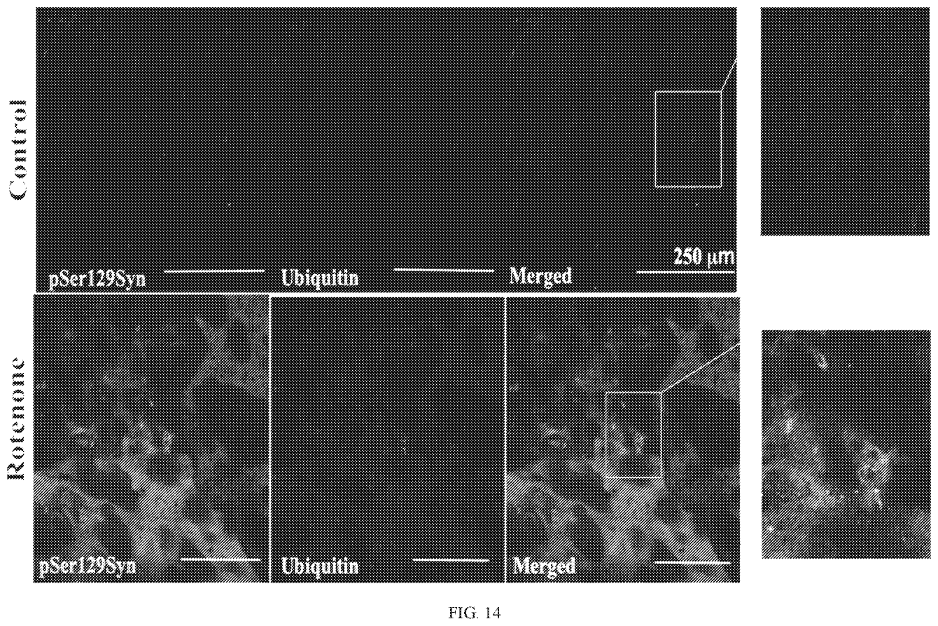

As the principal step towards moving the developed approach for modulating gene expression of SNCA via a DNA methylation-CRISPR/Cas9 tool forward into clinical setting, the capability of the SNCA-targeted LV-gRNA/dCas9-DNMT3A-2 system to reduce SNCA overexpression in a fine-tuned and precise manner was validated in the rats exposed to rotenone. Briefly, four Lewis rats, retired breeders at 6-9 months old, were treated with rotenone administered at 2.75-3.0 mg/mL via daily i.p. injections for the duration of 5 days. Control animals (n=4) received the vehicle (rotenone diluent) The SNCA expression levels were analyzed in the substantia nigra (SN), and the cerebellum as a control brain region. A significant increase in the levels of SNCA-mRNA (FIG. 13A) and protein (FIGS. 13B-13C) were found in the SN, amounting to >50% higher levels (P<0.05, student's 1-test). In the cerebellum, no increase in SNCA-mRNA was detected (FIG. 13A), while SNCA protein expression was moderately expression was moderately elevated (FIGS. 13B-13C). The therapeutic development was designed to target the regulation of SNCA transcription, therefore, the results of elevated SNCA expression at the mRNA levels demonstrate the suitability of the rotenone induced PD rat model for in vivo validation studies of the LV-gRNA-dCas9-DNMT3A system. The predominant modification of alpha-synuclein in Lewy body (LB) is phosphorylation on the serine residue at position 129 (pSer129Syn) which is a specific marker for all alpha-synuclein pathogenic aggregates. Thus, the reactivity to pSer129Syn was evaluated. Immunofluorescence (IF) analysis of the fixed brains using a PSer129 antibody showed an increase in pSer129Syn in the rats treated with rotenone compared to the control rats (FIG. 14).

Furthermore, inclusions (aggregations) of the phosphorylated alpha-synuclein were detected in the rats treated with rotenone and found evidence of co-localization of the phosphorylated alpha-synuclein with ubiquitin (FIG. 14). These results attest the feasibility of the PD rat model to capture pathologic phenotypes of PD. In summary, the PD animal model replicates key phenotypic aspects of PD and hence provides an excellent tool to test our system in vivo.

In attempting to correct the rotenone-induced overexpression of SNCA on the mRNA level, the rats were treated with viral particles delivered into SN by stereotaxic injections. Two weeks post-injections, the rats were treated with rotenone or the vehicle for 5 days.

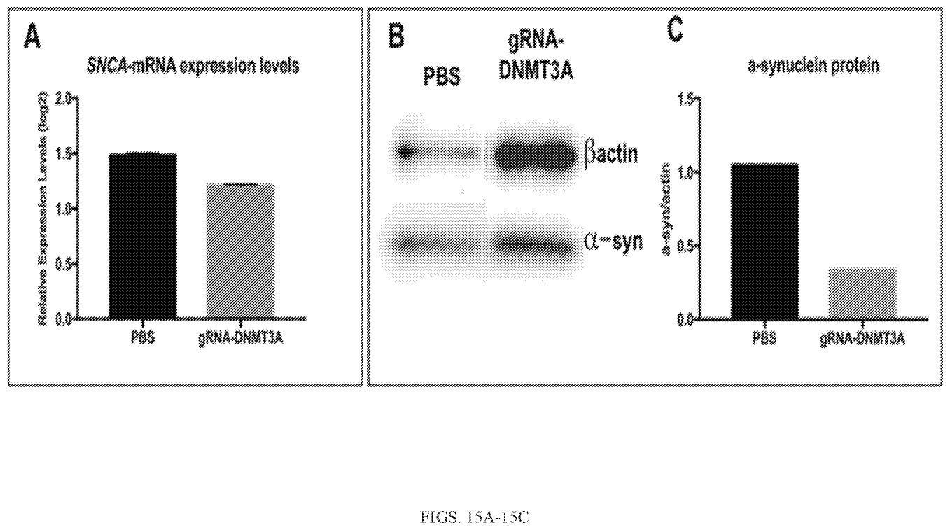

As described in FIG. 15A, the SNCA mRNA levels were augmented following the LV-gRNA-dCas9-DNMT3A delivery. The reduction in the alpha-synuclein expression levels by about 50% was demonstrated in the SN of the rats treated with the vector (2.5×10⁷ viral particles was used for the injections) (the SD bars were calculated per two animals from each groups injected either with PBS or the virus carried gRNA) (FIGS. 15B and 15C).

### Example 13

### Rescuing of Neuronal Nuclei PD Phenotype

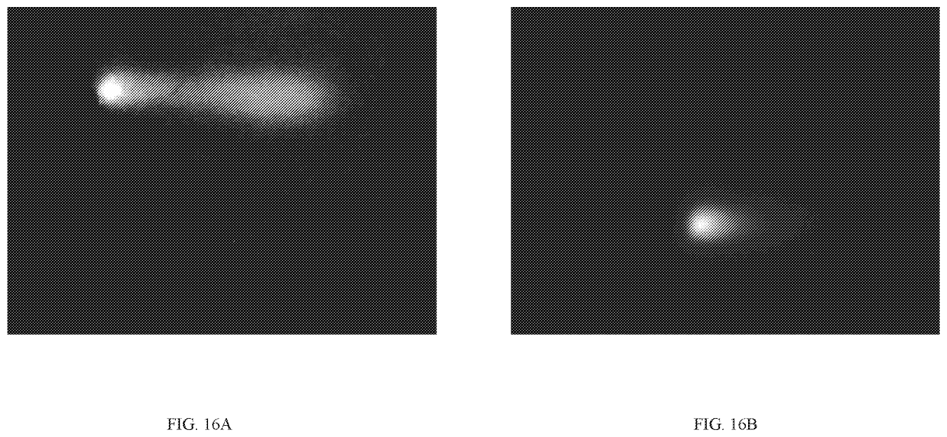

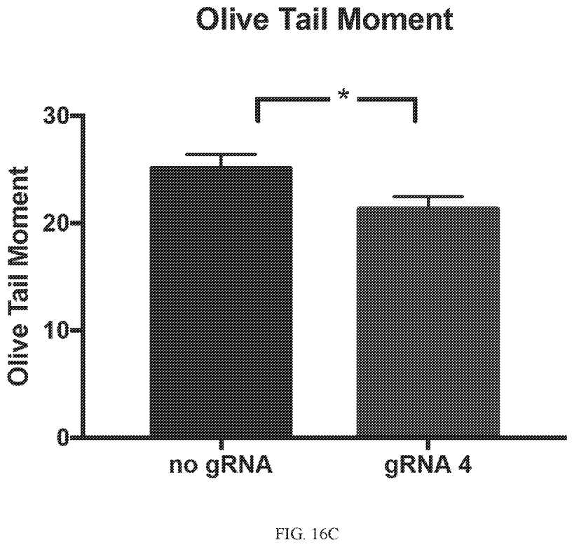

DNA damage was analyzed using the comet assay, specifically, measures of the Olive Tail Moment (OTM). The OTM is a comprehensive measure of DNA damage that includes the smallest detectable parts of migrating DNA as well as the number of broken DNA in the tail. The imaging was performed using a Zeiss Axio Observer Widefield Fluorescence Microscope, Germany. Comets were analyzed using the OpenComet Software, MA, USA; to determine the OTM, the parameter selected as the quantitative measure for each comet. The OTM was determined in 100 cells, 50 cells per each of two independent Comet experiments. The vector carrying gRNA 4 (gRNA4-dCas9-DNMT3A) showed a significant lower OTM value indicating it reversed the DNA damaged phenotype (FIGS. 16A-16C).

Overexpression (˜2-fold) of SNCA gene correlates with an exacerbation of aging-related phenotypes of the nuclear envelope Analysis of the nuclear circularity was performed using the Lamin B1 marker Nuclear circularity was quantified using the built-in ImageJ, circularity plug-in and assessed based on the Lamin B1 marker. A circularity value of 1.0 indicates a perfect circle. A value approaching 0 indicates an increasingly elongated polygon. Quantification of the nuclear envelope circularity demonstrated an increase in the nuclear envelope circularity in the NPC line that was transduced with gRNA4 versus no-grna control-vector. The data are plotted as frequency distributions of for 200 cells. n=2, One hundred cells per staining were analyzed for two independent experiments independent experiments, ****P 0.0001>according to Kolmogorov-Smirnov test Nuclear circularity was quantified using the built-in ImageJ. circularity plug-in and assessed based on the Lamin B1 marker. A circularity value of 1.0 indicates a perfect circle. A value approaching 0 indicates an increasingly elongated polygon. The data represents the mean of two independent experiments. The vector with gRNA 4 (gRNA4-dCas9-DNMT3A) showed a significant increase in the nuclear circularity indicating it rescued the phenotype of abnormal nuclei (FIGS. 17A-17C).

FIGS. 18A-18C show the analysis of the nuclear folding and bubbling using the Lamin A/C marker. The vector with gRNA 4 (gRNA4-dCas9-DNMT3A) showed a significant decrease in folded nuclei indicating it rescued the phenotype of abnormal nuclei shape.

### Example 14

### Protocol for Lentiviral Vector Design and Production

LVs represent an effective means of delivering CRISPR/dCas9 components for several reasons: (i) capacity to carry bulky DNA inserts, (ii) high-efficiency of transducing a broad range of cells including both dividing and non-dividing cells 30, (iii) ability to induce minimal cytotoxic and immunogenic responses.

Lentiviral platforms have a major advantage, over the most popular vector platform, adeno-associated vector (AAV), imprinted in the ability of the former to accommodate larger genetic inserts. AAVs can be generated at significantly higher yields but possess low packaging capacity (<4.8 kb) compromising their use for delivering all-in-one CRISPR/Cas9 systems. The protocol herein described further outlines the strategy to increase production and expression capabilities of the vectors, via modification in cis of the elements within the vector expression cassette. The strategy highlights the system's ability to produce viral particles in the range of 1010 viral units (VU)/mL.

TABLE 6
Table of Materials

| | | |
|---|---|---|
| Materials | Company | Catalog Number |

Equipment

| | | |
|---|---|---|
| Optima XPN-80 Ultracentrifuge | Beckman Coulter | A99839 |
| 0.22 μM filter unit, 1 L | Corning | 430513 |
| 0.45-μm filter unit, 500 mL | Corning | 430773 |
| 100 mm TC-Treated Culture Dish | Corning | 430167 |
| 15 mL conical centrifuge tubes | Corning | 430791 |
| 150 mm TC-Treated Cell Culture dishes | Corning | 353025 |
| with 20 mm Grid | | |
| 50 mL conical centrifuge tubes | Corning | 430291 |
| 6-well plates | Corning | 3516 |
| Aggrewell 800 | StemCell Technologies | 34811 |
| Allegra 25R tabletop centrifuge | Beckman Coulter | 369434 |
| BD FACS | Becton Dickinson | 338960 |
| Conical bottom ultracentrifugation tubes | Seton Scientific | 5067 |
| Conical tube adapters | Seton Scientific | PN 4230 |
| Eppendorf Cell Imaging Slides | Eppendorf | 30742060 |
| High-binding 96-well plates | Corning | 3366 |
| Inverted fluorescence microscope | Leica | DM IRB2 |
| QIAprep Spin Miniprep Kit (50) | Qiagen | 27104 |
| Reversible Strainer | StemCell Technologies | 27215 |
| SW32Ti rotor | Beckman Coulter | 369650 |
| VWR ® Disposable Serological Pipets, | VWR | 93000-694 |
| Glass, Nonpyrogenic | | |
| VWR ® Vacuum Filtration Systems | VWR | 89220-694 |
| xMark ™ Microplate Absorbance plate | Bio-Rad | 1681150 |
| reader | | |

Cell culture reagents

| | | |
|---|---|---|
| Human embryonic kidney 293T (HEK 293T) | ATCC | CRL-3216 |
| cells | | |
| Accutase | StemCell Technologies | 7920 |
| Anti-Adherence Rinsing Solution | StemCell Technologies | 7010 |
| Anti-FOXA2 Antibody | Abcam | Ab60721 |
| Anti-Nestin Antibody | Abcam | Abl8102 |
| Antibiotic-antimycotic solution, 100X | Sigma Aldrich | A5955-100ML |
| B-27 Supplement (50X), minus vitamin A | Thermo Fisher Scientific | 12587010 |
| BES | Sigma Aldrich | B9879 - BES |
| Bovine Albumin Fraction V (7.5% solution) | Thermo Fisher Scientific | 15260037 |
| CHIR99021 | StemCell Technologies | 72052 |
| Corning Matrigel hESC-Qualified Matrix | Corning | 08-774-552 |
| Cosmic Calf Serum | Hyclone | SH30087.04 |
| DMEM-F12 | Lonza | 12-719 |
| DMEM, high glucose media | Gibco | 11965 |
| DNeasy Blood & Tissue Kit | Qiagen | 69504 |
| EpiTect PCR Control DNA Set | Qiagen | 596945 |
| EZ DNA Methylation Kit | Zymo Research | D5001 |
| Gelatin | Sigma Aldrich | G1800-100G |
| Gentamicin | Thermo Fisher Scientific | 15750078 |
| Gentle Cell Dissociation Reagent | StemCell Technologies | 7174 |
| GlutaMAX | Thermo Fisher Scientific | 35050061 |
| Human Recombinant bFGF | StemCell Technologies | 78003 |
| Human Recombinant EGF | StemCell Technologies | 78006 |
| Human Recombinant Shh (C24II) | StemCell Technologies | 78065 |
| MEM Non-Essential Amino Acids | Thermo Fisher Scientific | 11140050 |
| Solution (100X) | | |
| mTeSR1 | StemCell Technologies | 85850 |
| N-2 Supplement (100X) | Thermo Fisher Scientific | 17502001 |
| Neurobasal Medium | Thermo Fisher Scientific | 21103049 |
| Non-Essential Amino Acid (NEAA) | Hyclone | SH30087.04 |
| PyroMark PCR Kit | Qiagen | 978703 |
| RPMI 1640 media | Thermo Fisher Scientific | 11875-085 |
| SB431542 | StemCell Technologies | 72232 |
| Sodium pyruvate | Sigma Aldrich | S8636-100ML |
| STEMdiff Neural Induction Medium | StemCell Technologies | 5835 |
| STEMdiff Neural Progenitor Freezing | StemCell Technologies | 5838 |
| Medium | | |
| TaqMan Assay FOXA2 | Thermo Fisher Scientific | Hs00232764 |
| TaqMan Assay GAPDH | Thermo Fisher Scientific | Hs99999905 |
| TaqMan Assay Nestin | Thermo Fisher Scientific | Hs04187831 |
| TaqMan Assay OCT4 | Thermo Fisher Scientific | Hs04260367 |
| TaqMan Assay PPIA | Thermo Fisher Scientific | Hs99999904 |
| Trypsin-EDTA 0.05% | Gibco | 25300054 |
| Y27632 | StemCell Technologies | 72302 |

p²⁴ ELISA reagents

| | | |
|---|---|---|
| Monoclonal anti-p²⁴ antibody | NIH AIDS Research and | 3537 |
| | Reference Reagent | |
| | Program | |
| Goat anti-rabbit horseradish peroxidase IgG | Sigma Aldrich | 12-348 |
| Working concentration 1:1500 | | |
| Goat serum, Sterile, 10 mL | Sigma | G9023 |
| Working concentration 1:1000 | | |
| HIV-1 standards | NIH AIDS Research and | SP968F |
| | Reference Reagent | |
| | Program | |
| Normal mouse serum, Sterile, 500 mL | Equitech-Bio | SM30-0500 |
| Polyclonal rabbit anti-p²⁴ antibody | NIH AIDS Research and | SP451T |
| | Reference Reagent | |
| | Program | |
| TMB peroxidase substrate | KPL | 5120-0076 |
| Working concentration 1:10,000 | | |

Plasmids

| | | |
|---|---|---|
| pMD2.G | Addgene | 12253 |
| pRSV-Rev | Addgene | 52961 |
| psPAX2 | Addgene | 12259 |

Restriction enzymes

| | | |
|---|---|---|
| BsmBI | New England Biolabs | R0580S |
| BsrGI | New England Biolabs | R0575S |
| EcoRV | New England Biolabs | R0195S |
| KpnI | New England Biolabs | R0142S |
| PacI | New England Biolabs | R0547S |
| SphI | New England Biolabs | R0182S |

Table 6 materials may be found in Tagliafierro L., et al. (J. Vis. Exp. 2019 Mar. 29:145).

Culturing HEK-293T Cells and Plating Cells for Transfection—NOTES: Human Embryonic Kidney 293T (HEK-293T) are cultured in complete high glucose DMEM (10% bovine calf serum, 1× antibiotic-antimycotic, Ix sodium pyruvate, lx non-essential amino acid, 2 mM L-Glutamine) at 37° C. 5/CO₂. For the reproducibility of the protocol, it is recommended to test calf serum when switching to a different lot/batch. Up to six 15 cm plates are needed for lentiviral production.

Use low passage cells to start a new culture (lower than passage 20). Once the cells reach 90-95% confluency, aspirate media and gently wash with sterile 1×PBS.

Add 2 mL of Trypsin-EDTA 0.05% and incubate at 37° C. for 3-5 min. To inactivate the dissociation reagent, add 8 mL of complete high glucose DMEM, and pipette 10-15 times with a 10 mL serological pipette to create a single cell suspension of 4×10 cells/mL.

For the transfections, coat 15 cm plates with 0.2% gelatin. Add 22.5 mL high glucose medium and seed the cells by adding 2.5 mL of cell suspension (total ˜1×107 cells/plate). Incubate plates at 37° C. with 5% CO₂ until 70-80% confluency is reached.

Transfecting HEK-293T Cells—Prepare 2×BES-buffered solution BBS and 1 M CaCl₂, according to 35. Filter solutions by passing it throughout a 0.22 μM filter and store at 4° C. The transfection mix has to be clear prior to its addition onto the cells. If the mix becomes cloudy during incubation, prepare fresh 2×BBS (pH=6.95).

To prepare the plasmid mix use the four plasmids as listed (the following mix is sufficient for one 15 cm plate: 37.5 μg of the CRISPR/dCas9-transfer vector (pBK492 (DNMT3A-PURO-NO-gRNA or pBK539, DNMT3A-GFP-NO-gRNA; 25 μg of pBK240 (psPAX2): 12.5 μg pMD2.G; 6.25 μg of pRSV-rev (FIG. 26A) Calculate volume of the plasmids based on the concentrations and add the required quantities into 15-ml conical tube. Add 312.5 μL 1 M CaCl2 and bring up to 1.25 mL final volume using sterile dd-H₂O. Gently add 1.25 mL of 2×BBS solution while vortexing the mix. Incubate for 30 min at room temperature. Cells are ready for transfection once they are 70-80% confluent.

Aspirate the media and replace it with 22.5 mL of freshly-prepared high glucose DMEM without serum. Add 2.5 mL of the transfection mixture dropwise to each 15-cm plate. Swirl the plates and incubate at 37° C. with 5% CO2 for 2-3 h.

After 3 h, add 2.5 mL (10%) serum per plate and incubate overnight at 37° C. 5% CO₂.

Day 1 after transfection—1 d after transfection, observe the cells to ensure that there is no or minimal cell death, and that the cells formed a confluent culture (100%) Change media by adding 25 mL of freshly-prepared high glucose DMEM+10% serum to each plate.

Incubate at 37° C. 5% CO₂ for 48 h.

Harvesting Virus—Collect the supernatant from all the transfected cells and pool in 50 mL conical tubes. Centrifuge at 400-450× g for 10 min. Filter the supernatant through a 0.45 μm vacuum filter unit. After filtration, the supernatant can be kept at 4° C. for short-term storage (up to 4 days). For long-term storage, prepare aliquots and store the aliquots at −80° C.

NOTE: The non-concentrated viral preparations are expected to be ˜2-3×10⁷ vu/mL (see herein for titer determination). It is highly recommended to prepare single-use aliquots, since multiple freeze-thaw cycles will result in a 10-20% loss in functional titers.

Concentration of Viral Particles—NOTE: For the purification, a two steps double-sucrose method involving a sucrose gradient step and a sucrose cushion step is performed (FIG. 26B).

To create a sucrose gradient, prepare the conical ultracentrifugation tubes in the following order: 0.5 mL 70% sucrose in 1×PBS, 0.5 mL 60% sucrose in DMEM, 1 mL 30% sucrose in DMEM, 2 mL 20% sucrose in 1×PBS.

Carefully, add the supernatant, collected in Step 1.4, to the gradient. Since the total volume collected from four 15 cm plates is 100 mL, use six ultracentrifugation tubes to process the viral supernatant.

Equally distribute viral supernatant among each ultracentrifugation tube. To avoid tube breakage during centrifugation, fill ultracentrifugation tubes to at least three-fourths their total volume capacity. Balance the tubes with 1×PBS Centrifuge samples at 70,000×g for 2 h at 17° C.

NOTE: To maintain the sucrose layer during the acceleration and deceleration steps, allow the ultracentrifuge to slowly accelerate and decelerate the rotor from 0 to 200 g and from 200 g to 0 during the first and last 3 min of the spin, respectively.

Gently collect 30-60% sucrose fractions into clean tubes. Add 1×PBS (cold) up to 100 ml of total volume. Mix by pipetting multiple times

Carefully, stratify the viral preparation on a sucrose cushion by adding 4 mL of 20% sucrose (in 1×PBS) to the tube. Continue by pipetting ˜20-25 mL of the viral solution per each tube. Fill with 1×PBS, if the volume of the tubes is less than three-fourths. Carefully balance the tubes. Centrifuge at 70.000× g for 2 h at 17° C. Empty the supernatant and invert the tubes on paper towels to allow the remaining liquid to drain.

Remove all the liquid by cautiously aspirating the remaining liquid. At this step, pellets containing the virus is barely visible as small translucent spots. Add 70 μL of 1×PBS to the first tube to resuspend the pellet. Thoroughly pipette the suspension and transfer it to the next tube until all pellets are resuspended.

Wash the tubes with additional 50 μL 1×PBS and mix as before. At this step, the volume of the final suspension is ˜120 μL and appears slightly milky. To obtain a clear suspension, proceed with a 60 s centrifugation at 10,000×g. Transfer the supernatant to a new tube, make 5 μL aliquots, and store them at −80° C.

NOTE: Lentiviral vector preparations are sensitive to the repeated cycles of freezing and thawing. In addition, it is suggested that the remaining steps are done in tissue-culture containment, or designated areas qualified in terms of being at adequate levels of biosafety standards. (FIG. 26B).

Quantification of Viral Titers—NOTE: The estimation of viral titers is performed using the p24-enzyme-linked immunosorbent assay (ELISA) method (p24gag ELISA) and according to the NIH AIDS Vaccine Program protocol for HIV-1 p24 Antigen Capture Assay, with slight modifications.

Use 200 μL of 0.05/Tween 20 in cold 1×PBS (PBS-T) to wash three times the wells of a 96 well plate.

To coat the plate, use 100 μL of monoclonal anti-p24 antibody diluted 1:1500 in 1×PBS Incubate the plate overnight at 4° C.

Prepare blocking reagent (1% BSA in 1×PBS) and add 200 μL to each well to avoid non-specific binding. Use 200 μL PBS-T to wash the well three times for at least 1 h at room temperature.

Proceed with samples preparation: when working with concentrated vector preparations dilute vector 1:100 by using 1 μL of the sample, 89 μL of dd-H20, and 10 μL of Triton X-100 (final concentration of 10%) For non-concentrated preparations, dilute samples 1:10.

Obtain HIV-1 standards by using a 2-fold serial dilution (starting concentration is 5 ng/mL).

Dilute concentrated samples (prepared in Step 16.4) in RPMI 1640 supplemented with 0.2% Tween 20 and 1% BSA to obtain 1:10,000, 1:50,000, and 1:250,000 dilutions. Similarly, dilute non-concentrated samples (prepared in Step 1.6.4) in RPMI 1640 supplemented with 0.2% Tween 20 and 1% BSA to establish 1:500, 1:2500, and 1:12,500 dilutions.

Add samples and standards on the plate in triplicates. Incubate overnight at 4° C.

The next day, wash the wells six times.

Add 100 μL polyclonal rabbit anti-p24 antibody, diluted 1:1000 in RPMI 1640, 10% FBS, 0.25% BSA, and 2% normal mouse serum (NMS) and incubate at 37° C. for 4 h.

Wash the wells six times. Add goat anti-rabbit horseradish peroxidase IgG diluted 1:10,000 in RPMI 1640 supplemented with 5% normal goat serum, 2% NMS, 0.25% BSA, and 0.01% Tween 20. Incubate at 37° C. for 1 h.

Wash the well six times. Add TMB peroxidase substrate and incubate at room temperature for 15 min.

To stop the reaction, add 100 μL of 1 N HCL. In a microplate reader, measure absorbance at 450 nm.

Measurement of fluorescent reporter intensity—Use the viral suspension to obtain a ten-fold serial dilution (from 10⁻¹ to 10⁻⁵) in 1×PBS.

Plate 5×10⁵ HEK-293T cells in each well of a 6-well plate. Apply 10 μL of each viral dilution to the cells and incubate at 37° C. 5% CO₂ for 48 h.

Proceed to the Fluorescence Activated Cell Sorting (FACS) analysis as follows: detach cells by adding 200 μL of 0.05% Trypsin-EDTA solution Incubate cells at 37° C. for 5 min and resuspend them in 2 mL of DMEM medium (with serum). Collect samples into a 15 mL conical tube and centrifuge at 400 g at 4° C. Resuspend the pellet in 500 μL of cold 1×PBS.

Fix cells by adding 500 μl of 4% PFA and incubate for 10 min at room temperature.

Centrifuge at 400 g at 4° C. and resuspend the pellet in 1 mL of 1×PBS. Analyze GFP expression using a FACS instrument.

To determine the virus functional titer, use the following formula:

Transducting units (TU) per nL=Tg/Tn×N×1000/V

Tg=number of GFP-positive cells, Tn=total number of cells; N=total number of transduced cells; V=volume used for transduction (in μL).

Counting GFP-positive cells—NOTE Determine the Multiplicity of Infection (MOI) that is employed for transduction Test a wide range of MOIs (from MOI=1 to MOI=:10)

Seed 3-4×10⁵ HEK-293T cells per each well of a 6 well plate.

When cells reach >80% confluency, transduce with the vector at the MOI-of-interest.

Incubate at 37° C., 5% CO₂, and monitor the GFP signal in the cells for 1-7 days.

Count the number of GFP-positive cells. Employ a fluorescent microscope (PLAN 4× objective, 0.1 N. A, 40× magnification) using a GFP filter (excitation wavelength. 470 nm, emission wavelength: 525 nm). Use untransduced cells to set the control population of GFP-negative cells.

Employ the following formula to determine the functional titer of the virus.

Transducting units (TU) per mL=(N)×(D)×(M)×V

NOTE: N=number of GFP-positive cells, D=dilution factor, M=magnification factor V=volume of virus used for transduction. Calculate results following this example for the calculation: for 10 GFP-positive cells (N) counted at a dilution (D) of 10⁻⁴ (1:10,000) at 20× magnification (M) in a 10 μL sample (V), the TU per mL will be (10×10⁴)×(20)×(10)×(100)=2×10⁸ vu/mL.

MD NPCs Differentiation

Culturing hiPSCs—NOTE: Human Induced Pluripotent Stem Cells (hiPSCs) from a patient with the triplication of the SNCA locus, ND34391, were obtained from the NINDS catalogue (See Table 6).

Culture hiPSCs under feeder-independent condition in feeder-free ESC-iPSC culture medium (See Table 6) onto hESC-qualified basic matrix membrane (BMM)-coated plates (See Table 6). Wash confluent colonies with 1 mL DMEM-F12, add 1 mL of dissociation reagent (see Table 6), and incubate for 3 min at room temperature.

Aspirate the dissociation reagent and add 1 mL of feeder-free ESC-iPSC culture medium.

Scrape plate using a cell lifter and resuspend colonies in 11 mL of feeder-free ESC-iPSC culture medium by pipetting 4-5 times using borosilicate pipettes.

Plate 2 mL of colony suspension onto BMM-coated plates and place the plate at 37° C. 5% CO₂. Perform a daily medium change and split cells every 5-7 d.

Differentiation into MD NPCs—NOTE: The differentiation of hiPSCs into Dopaminergic Neural Progenitor Cells (MD NPCs), has been performed using a commercially-available Neural Induction Medium protocol per manufacturers' instructions, with slight modifications (see Table 6). The 1st d of the differentiation is considered as day 0. High-quality hiPSCs are required for efficient neural differentiation. The induction of MD NPCs was performed as using an embryoid body (EB)-based protocol.

Prior to start the differentiation of hiPSCs, prepare microwell culture plates (see Table 6) according to manufacturers' instructions.

After preparing the microwell culture plate, add 1 mL of Neural Induction Medium (NIM, see Table 6) supplemented with 10 μM of Y-27632.

Set the plate aside until ready to use.

Wash hiPSCs with DMEM-F12, add 1 mL cell detachment solution (see Table 6), and incubate 5 min at 37° C. 5% CO₂.

Resuspend single cells in DMEM-F12 and centrifuge at 300 g for 5 min.

Carefully aspirate supernatant and resuspend cells in NIM+10 μM Y-27632 to obtain a final concentration of 3×10⁶ cells/mL.

Add 1 mL of the single-cell suspension to a single well of the microwell culture plate and centrifuge the plate at 100 g for 3 min.

Examine the plate under the microscope to ensure even distribution of the cells among microwell and incubate cells at 37° C. 5% CO₂.

Day 1-day 4—Perform a daily partial medium change.

Using a 1 mL micropipette, remove 1.5 mL of the medium and discard. Slowly, add 1.5 mL of fresh NIM without Y-27632.

Repeat step 2.2.10 until day 4.

Day 5: Coat 1 well of a 6-well plate with BMM.

Place a 37 μm Reversible Strainer (see Table 6) on top of a 50 mL conical tube (waste). Point the arrow of the reversible strainer upwards.

Remove the medium from the microwell culture plate without disturbing the formed EBs.

Add 1 mL of DMEM-F12 and promptly collect the EBs with the borosilicate pipette and filter through the strainer.

Repeat steps until all EBs are removed from the microwell culture plate.

Invert the strainer over a new 50 mL conical tube and add 2 mL of NIM to collect all the EBs.

Plate 2 mL of the EBs suspension into a single well of the BMM-coated plate using a borosilicate pipette. Incubate EBs at 37° C. 5% CO₂.

Day 6: Prepare 2 mL of NIM+200 ng/mL SHH (See Table of Material) and perform a daily medium change.

Day 8: Examine the percentage of neuronal induction.

Count all attached EBs and specifically determine the number of each individual EB that is filled with neural rosettes. Quantify neural rosette induction using the following formula:

$$\frac{\# of EBs with \geq 50 \% neural rosettes}{Total \# of EBs} \times 100$$

Note: If neural induction is <75% neural rosette selection may be inefficient.

Day 12: Prepare 250 mL of N2B27 medium as follows 119 mL Neurobasal Medium, 119 mL DMEM/F12 Medium, 2.5 mL Glutamax, 2.5 mL NEAA, 2.5 mL N2 supplement, 5 mL B27 without Vitamin A, 250 μL Gentamicin 50 mg/mL, 19.66 ?l BSA 7 mg/mL.

To prepare 50 mL of complete N2B27 medium add 3 μM CHIR99021, 2 μM SB431542, 20 ng/mL bFGF, 20 ng/mL EGF. and 200 ng/mL SHH.

Note: It is important to prepare completed medium right before use.

Aspirate medium from the wells containing the neural rosettes and wash with 1 mL of DMEM-F12.

Ad 1 mL of Neural Rosette Selection Reagent (see Table 6) and incubate at 37° C. 5% CO₂ for 1 h.

Remove the Selection Reagent and using a 1 mL pipettor aim directly at the rosette clusters.

Add the suspension to a 15 mL conical tube, and repeat (remove the Selection Reagent and using a 1 mL pipettor aim directly at the rosette clusters and add to canonical tube) until the majority of the neural rosette clusters have been collected.

Note: To avoid contamination with non-neuronal cell-types, do not over-select.

Centrifuge rosette suspension at 350 g for 5 min Aspirate supernatant and resuspend the neural rosettes in N2B27+200 ng/mL SHH. Add neural rosette suspension to a BMM-coated well and incubate the plate at 37° C. 5% CO₂.

Day 13-day 17. Perform a daily medium change using completed N2B27 medium. Passage cells when cultures are 80-90 confluency.

To split cells, prepare a BMM-Coated Plate.

Wash cells with 1 mL DMEM-F12, aspirate medium and add 1 mL dissociation reagent (See Table 6).

Incubate for 5 min at 37° C., add 1 mL of DMEM-F12 and dislodge attached cells by pipetting up and down. Collect NPC suspension to a 15 mL conical tube. Centrifuge at 300 g for 5 min.

Aspirate supernatant and resuspend cells in 1 mL of complete N2B27+200 ng/mL SHH.

Count cells and plate at a density of 1.25×10⁵ cells/cm² and incubate cells at 37° C. 5% CO₂.

Change medium every other day using complete N2B27+200 ng/mL SHH.

Note: At this passage, NPCs are considered Passage P0. SHH can be withdrawn from the N2B27 medium at P2

Passage cells once they reach 80-90% confluency.

At this stage, confirm that cells express Nestin and FoxA2 markers by using immunocytochemistry and qPCR. This protocol leads to the generation of 85% double-positive cells for the Nestin and FoxA2 markers.

For passaging cells, repeat steps in paragraphs [00318]-[00324] Freeze cells starting from passage P2 For freezing cells, repeat steps 2. [00318]-[00324] and resuspend cell pellet at 2-4×10⁶ cells/mL using cold Neural Progenitor Freezing Medium (see Table 6).

Transfer 1 mL of cell suspension into each cryovial and freeze cells using a standard slow-rate controlled cooling system. For long term storage, keep cells in liquid-nitrogen.

Thawing MD NPCs—Prepare BMM-coated plate and warm complete N2B27. Add 10 mL of warm DMEM-F12 to a 15 mL conical tube. Place cryovial in a 37° C. heat block for 2 min.

Transfer cells from the cryovial to the tube containing DMEM-F12. Centrifuge 300 g for 5 min.

Aspirate the supernatant, resuspend cells in 2 mL N2B27, and add cell suspension to 1 well of a BMM-coated plate. Incubate cells at 37° C. 5% CO₂.

Transduction of MD NPCs and analysis of methylation changes.

Transduction of MD NPCs.

Transduce MD NPCs at 70% confluency with LV-gRNA/dCas9-DNMT3A vectors at the multiplicity of infections (MOIs)=2. Replace N2B27 medium 16 h post-transduction.

48 h post transduction add N2B27 media supplemented with from 1 to 5 μg/mL puromycin to obtain the stable MD NPC-lines. Cells are ready for downstream applications (DNA, RNA, protein analyses, and phenotypic characterization, freezing and passaging as described herein.)

Differentiation of MD NPCs. The EB-based protocol described herein, allows the differentiation of MD NPCs. See Tagliafierro, L., et al., J. Vis. Exp. 2019 Mar. 29: 145. This differentiation protocol produces 83.3% of cells double positive for the Nestin and FOXA2 markers, confirming the successful differentiation of these cells.

Validation of the pyrosequencing assays for the SNCA-intron1 methylation profile. Seven pyrosequencing assays were established to evaluate the DNA methylation status in the SNCA intron 1 See Kantor et al., Mol. Ther. 2018: Nov. 7:26(11): 2638-2649. The Chr4: 89,836,150-89,836,593 (GRCh38/hg38) region contains 23 CpGs. The designed assays were validated for linearity using different mixtures of unmethylated (U) and methylated (M) bisulfite converted DNAs as standards. Mixtures were used in the following ratios: 100 U:0M, 75 U:25M, 50 U:50M, 25 U:75M, 0 U:100M. All seven assays were validated and showed linear correlation R2>0.93). Using the validated assays, we were able to determine the methylation levels at the 23 CpGs in the SNCA intron 1 treated and untreated with gRNA 1-4 vectors (FIG. 3).

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art Such changes and modifications, including without limitation those relating to the chemical structures. substituents, derivatives, intermediates, syntheses, compositions, formulations, or methods of use of the invention, may be made without departing from the spirit and scope thereof.

For reasons of completeness, various aspects of the invention are set out in the following numbered clause:

Clause 1. A composition for epigenome modification of a VNA gene, the composition comprising: (a)(i) a fusion protein or (a)(ii) a nucleic acid sequence encoding a fusion protein, the fusion protein comprising two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, deacetylase activity, or combination thereof, and (b)(i) at least one guide RNA (gRNA) or (b)(ii) a nucleic acid sequence encoding at least one guide gRNA, wherein the at least one gRNA targets the fusion protein to a target region within the SNCA gene.

Clause 2. The composition of clause 1, wherein the at least one gRNA targets the fusion protein to a target region within intron 1 of the SNCA gene.

Clause 3. The composition of clause 2, wherein the composition modifies at least one CpG island region within intron 1 of the SNCA gene.

Clause 4. The composition of clause 3, wherein the at least one CpG island region comprises CpG1, CpG2, CpG3, CpG4, CpG5, CpG6, CpG7, CpG8, CpG9, CpG10, CpG11, CpG12, CpG13, CpG14, CpG15, CpG16, CpG17, CpG18, CpG19, CpG20, CpG21, CpG22, CpG23, or a combination thereof.

Clause 5 The composition of clause 3 or 4, wherein the at least one CpG island region comprises CpG1, CpG3, CpG6, CpG7, CpG8, CpG9, CpG18, CpG19, CpG20, CpG21, CpG22, or a combination thereof.

Clause 6. The composition of any one of clauses 3-5, wherein the second polypeptide domain comprises a peptide having methylase activity and the fusion protein methylates at least one CpG island region within intron 1 of the SNCA gene.

Clause 7 The composition of any one of clauses 1-6, wherein the at least one gRNA comprises a polynucleotide sequence of at least one of SEQ ID NO: 2, SEQ ID NO: 3, SEQ ID NO: 4, SEQ ID NO: 5, complement thereof, variant thereof, or a combination thereof.

Clause 8. The composition of clause 1, wherein the at least one gRNA targets the fusion protein to a target region within intron 4 of the SNCA gene, and optionally, wherein the target region within intron 4 is a H3K4Me3, H3K4Me1 and/or H3K27Ac mark.

Clause 9. The composition of any one of clauses 1-8, wherein the second polypeptide domain comprises DNA (cytosine-5)-methyltransferase 3A (DNMT3A), a functional fragment thereof, and/or a variant thereof.

Clause 10. The composition of any one of clauses 1-9, wherein the fusion protein represses the transcription of the SNCA gene.

Clause 11. The composition of any one of clauses 1-10, wherein the Cas protein comprises a Cas9 endonuclease having at least one amino acid mutation which knocks out nuclease activity of Cas9.

Clause 12. The composition of clause 11, wherein the at least one amino acid mutation is at least one of D10A and H840A.

Clause 13. The composition of clause 11 or 12, wherein the Cas protein comprises an amino acid sequence of SEQ ID NO: 10.

Clause 14. The composition of any one of clauses 1-13, wherein the second polypeptide domain is fused to the C-terminus, N-terminus, or both, of the first polypeptide domain.

Clause 15. The composition of any one of clauses 1-14, further comprising a nuclear localization sequence.

Clause 16 The composition of anyone of clauses 1-15, further comprising a linker connecting the first polypeptide domain to the second polypeptide domain.

Clause 17 The composition of anyone of clauses 1-16, wherein the second polypeptide domain comprises an amino acid sequence of SEQ ID NO: 11.

Clause 18 The composition of any one of clauses 1-17, wherein the fusion protein comprises an amino acid sequence of SEQ TD NO: 13.

Clause 19 The composition of anyone of clauses 1-18, wherein the fusion protein is encoded by a polynucleotide sequence comprising a polynucleotide sequence of SEQ ID NO 14.

Clause 20 The composition of anyone of clauses 1-19, comprising administering to, or provided in, the subject any of (a)(ii) and (b)(ii), (a)(i) and (b)(i), (a)(i) and (b)(ii), or (a)(ii) and (b)(i).

Clause 21. The composition of any one of clauses 1-20, wherein the nucleic acid of (a)(ii) and/or (b)(ii) comprises DNA or RNA.

Clause 22. The composition of any one of clauses 1-21, wherein one or both of (a) and (b) are packaged in a viral vector.

Clause 23. The composition of any one of clauses 1-22, wherein (a) and (b) are packaged in the same viral vector.

Clause 24. The composition of clause 22 or 23, wherein the viral vector comprises a lentiviral vector.

Clause 25. The composition of any one of clauses 22-24, wherein the viral vector comprises an episomal integrase-deficient lentiviral vector (IDLV) or an episomal integrase-competent lentiviral vector (ICLV).

Clause 26. The composition of any one of clauses 22-25, wherein the viral vector comprises a polycistronic-protein composition comprising multiple promoters, p2a; t2a; IRES, or combinations thereof.

Clause 27 An isolated polynucleotide encoding the composition of any one of clauses 1-26.

Clause 28. A vector comprising the isolated polynucleotide of clause 27.

Clause 29. The vector of clause 28, wherein the vector is a viral vector.

Clause 30. The vector of clause 28 or 29, wherein the viral vector is a lentiviral vector.

Clause 31 The vector of any one of clauses 28-30, wherein the viral vector is an episomal integrase-deficient lentiviral vector (IDLV) or an episomal integrase-competent lentiviral vector (ICLV).

Clause 32. A host cell comprising the isolated polynucleotide of clause 27 or the vector of any one of clauses 28-31.

Clause 33. A pharmaceutical composition comprising at least one of the composition of clauses 1-26, the isolated polynucleotide of clause 27, the vector of any one of clauses 28-31, the host cell of clause 32, or combinations thereof.

Clause 34. A kit comprising at least one of the composition of clauses 1-26, the isolated polynucleotide of clause 27, the vector of any one of clauses 28-31, or combinations thereof.

Clause 35. A method of in vivo modulation of expression of a SNCA gene in a cell or a subject the method comprising contacting the cell or subject with at least one of the composition of clauses 1-26, the isolated polynucleotide of clause 27, the vector of any one of clauses 28-31, the pharmaceutical composition of clause 33, or combinations thereof, in an amount sufficient to modulate expression of the gene.

Clause 36. A method of treating a disease or disorder associated with elevated SN-4 expression levels in a subject, the method comprising administering to the subject or a cell in the subject at least one of the composition of clauses 1-26, the isolated polynucleotide of clause 27, the vector of any one of clauses 28-31, the pharmaceutical composition of clause 33, or combinations thereof.

Clause 37. A method of in vivo modulating expression of a SNCA gene in a cell or a subject, the method comprising contacting the cell or subject with: (a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b)(i) at least one guide RNA (gRNA) that targets the fusion molecule to a target region within the SNCA gene or (b)(ii) a nucleic acid sequence encoding at least one gRNA that targets the fusion protein to a target region within the SNCA gene, in an amount sufficient to modulate expression of the gene.

Clause 38. A method of treating a disease or disorder associated with elevated SNCA expression levels in a subject, the method comprising administering to the subject or a cell in the subject: (a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity: and (b)(i) at least one guide RNA (gRNA) that targets the fusion molecule to a target region within the SNCA gene or (b)(ii) a nucleic acid sequence encoding at least one gRNA that targets the fusion molecule to a target region within the SNCA gene, in an amount sufficient to modulate expression of the gene.

Clause 39. The method of clause 37 or 38, wherein the at least one gRNA or nucleic acid sequence encoding the at least one gRNA targets the fusion protein to a target region within intron 1 of the SNCA gene.

Clause 40. The method of clause 39, wherein the fusion protein modifies at least one CpG island region within intron 1 of the SNCA gene.

Clause 41. The method of clause 40, wherein the at least one CpG island region comprises CpG1, CpG2, CpG3, CpG4, CpG5, CpG6, CpG7, CpG8, CpG9, CpG10, CpG11, CpG12, CpG13, CpG14, CpG15, CpG16, CpG17, CpG18, CpG19, CpG20, CpG21, CpG22, CpG23, or a combination thereof.

Clause 42 The method of clause 40 or 41, wherein the at least one CpG island region comprises CpG1, CpG3, CpG6, CpG7, CpG8, CpG9, CpG18, CpG19, CpG20, CpG21, CpG22, or a combination thereof.

Clause 43. The method of any one of clauses 40-42, wherein the second polypeptide domain comprises a peptide having methylase activity and the fusion protein methylates at least one CpG island region within intron 1 of the SNCA gene.

Clause 44. The method of any one of clauses 37-43, wherein the at least one gRNA comprises a polynucleotide sequence of at least one of SEQ ID NO: 2, SEQ ID NO: 3, SEQ ID NO: 4, SEQ ID NO: 5, complement thereof, variant thereof, or a combination thereof.

Clause 45 The method of clause 37 or 38, wherein the at least one gRNA or nucleic acid sequence encoding the at least one gRNA targets the fusion protein to a target region within intron 4 of the SNCA gene, and optionally, wherein the target region within intron 4 is a H3K4Me3. H3K4Me1 and/or H3K27Ac mark.

Clause 46 The method of any one of clauses 37-45. wherein the second polypeptide domain comprises DNA (cytosine-5)-methyltransferase 3A (DNMT3A), a functional fragment thereof, and/or a variant thereof.

Clause 47. The method of any one of clauses 37-46, wherein the fusion protein represses the transcription of the SNCA gene.

Clause 48. The method of any one of clauses 37-47, wherein the Cas protein comprises a Cas9 endonuclease having at least one amino acid mutation which knocks out nuclease activity of Cas9.

Clause 49. The method of clause 48, wherein the at least one amino acid mutation is at least one of D10A and H840A.

Clause 50. The method of clause 48 or 49, wherein the Cas protein comprises an amino acid sequence of SEQ ID NO: 10.

Clause 51. The method of anyone of clauses 37-50, wherein the second polypeptide domain is fused to the C-terminus, N-terminus, or both, of the first polypeptide domain.

Clause 52. The method of anyone of clauses 37-51, further comprising a nuclear localization sequence.

Clause 53. The method of any one of clauses 37-52, further comprising a linker connecting the first polypeptide domain to the second polypeptide domain.

Clause 54. The method of any one of clauses 37-53, wherein the second polypeptide domain comprises an amino acid sequence of SEQ ID NO: 11.

Clause 55. The method of any one of clauses 37-54, wherein the fusion protein comprises an amino acid sequence of SEQ ID NO: 13.

Clause 56 The method of any one of clauses 37-55, wherein the fusion protein is encoded by a polynucleotide sequence comprising a polynucleotide sequence of SEQ ID NO: 14.

Clause 57 The method of anyone of clauses 37-56, comprising administering to, or provided in, the subject any of: (a)(ii) and (b)(ii), (a)(i) and (b)(i), (a)(i) and (b)(ii), or (a)(ii) and (b)(i).

Clause 58. The method of any one of clauses 37-57, wherein the nucleic acid of (a)(ii) and/or (b)(ii) comprises DNA or RNA

Clause 59. The method of any one of clauses 37-58, wherein one or both of (a) and (b) are packaged in a viral vector.

Clause 60. The method of any one of clauses 37-59, wherein (a) and (b) are packaged in the same viral vector.

Clause 61. The method of clause 59 or 60, wherein the viral vector comprises a lentiviral vector.

Clause 62. The method of any one of clauses 59-61, wherein the viral vector comprises an episomal integrase-deficient lentiviral vector (IDLV) or an episomal integrase-competent lentiviral vector (ICLV).

Clause 63. The method of any one of clauses 35-62, wherein the cell comprises SNCA gene triplication (SNCA-Tri), wherein the levels of SNCA are elevated compared to physiological levels in a control cell that does not have SNCA-Tri.

Clause 64. The method of clause 63, wherein the SNCA levels are reduced to physiological levels after administering or providing any one of (a)(ii) and (b)(ii), (a)(i) and (b)(i), (a)(i) and (b)(ii), or (a)(ii) and (b)(i) to the subject or cell in the subject.

Clause 65. The method of any one of clauses 35-64, wherein the expression of the SNCA gene is reduced by at least 20%.

Clause 66. The method of any one of clauses 35-65, wherein the expression of the SNCA gene is reduced by at least 90%.

Clause 67. The method of any one of clauses 35-66, wherein levels of α-synuclein are reduced by at least 25%.

Clause 68. The method of any one of clauses 35-67, wherein levels of α-synuclein are reduced by at least 36%.

Clause 69 The method of any one of clauses 35-68, wherein mitochondrial superoxide production is reduced by at least 25% and/or cell viability is increased at least 1.4 fold.

Clause 70. The method of any one of clauses 36 or 38-69, wherein the disease or disorder is a neurodegenerative disorder.

Clause 71. The method of clause 70, wherein the neurodegenerative disorder is a SNCA-related disease or disorder.

Clause 72. The method of clause 70 or 71, wherein the neurodegenerative disorder is a synucleinopathy.

Clause 73. The method of any one of clauses 70-72, wherein the neurodegenerative disorder is Parkinson's disease or dementia with Lewy bodies.

Clause 74. The method of any one of clauses 35-73, wherein the cell is a dopaminergic (ventral midbrain) Neural Progenitor Cell (MD NPC), a midbrain dopaminergic neuron (mDA) or a basal forebrain cholinergic neuron (BFCN).

Clause 75. The method of any one of clauses 35-74, wherein the subject is a mammal.

Clause 76. The method of any one of clauses 35-75, wherein the subject is a human or a murine subject.

Clause 77. The method of any one of clauses 35-76, wherein the viral vector comprises a polycistronic-protein composition comprising multiple promoters, p2a; t2a; IRES, or combinations thereof.

Clause 78. A viral vector system for epigenemic editing, the viral vector system comprising: (a) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b) a nucleic acid sequence encoding at least one guide RNA (gRNA) that targets the fusion protein to a target region within the SNCA gene.

Clause 79 The viral vector system of clause 78, wherein the at least one gRNA targets the fusion protein to a target region within intron 1 of the SNCA gene.

Clause 80 The viral vector system of clause 79, wherein the fusion protein modifies at least one CpG island region within intron 1 of the SNCA gene.

Clause 81 The viral vector system of clause 80, wherein the at least one CpG island region comprises CpG1, CpG2, CpG3, CpG4, CpG5, CpG6, CpG7, CpG8, CpG9, CpG10, CpG11, CpG12, CpG13, CpG14, CpG15, CpG16, CpG17, CpG18, CpG19, CpG2, CpG21. CpG22, CpG23, or a combination thereof.

Clause 82 The viral vector system of clause 80 or 81, wherein the at least one CpG island region comprises CpG1, CpG3, CpG6, CpG7, CpG8, CpG9, CpG18. CpG19, CpG20, CpG21, CpG22, or a combination thereof.

Clause 83. The viral vector system of any one of clauses 80-82, wherein the second polypeptide domain comprises a peptide having methylase activity and the fusion protein methylates at least one CpG island region within intron 1 of the SNA gene.

Clause 84. The viral vector system of any one of clauses 78-83, wherein the at least one gRNA comprises a polynucleotide sequence of at least one of SEQ ID NO: 2, SEQ ID NO. 3, SEQ ID NO: 4, SEQ ID NO: 5, complement thereof, variant thereof, or a combination thereof.

Clause 85. The viral vector system of clause 78, wherein the at least one gRNA targets the fusion protein to a target region within intron 4 of the SNCA gene, and optionally, wherein the target region within intron 4 is a H3K4Me3, H3K4Me1 and/or H3K27Ac mark.

Clause 86. The viral vector system of any one of clauses 78-85, wherein the second polypeptide domain comprises DNA (cytosine-5)-methyltransferase 3A (DNMT3A), a functional fragment thereof, and/or a variant thereof.

Clause 87. The viral vector system of any one of clauses 78-86, wherein the second polypeptide domain comprises an amino acid sequence of SEQ ID NO:11.

Clause 88 The viral vector system of any one of clauses 78-87, wherein the Cas protein comprises a Cas9 endonuclease having at least one amino acid mutation which knocks out nuclease activity of Cas9.

Clause 89. The viral vector system of clause 88, wherein the at least one amino acid mutation is at least one of D10A and H840A.

Clause 90 The viral vector system of clause 88 or 89, wherein the Cas protein comprises an amino acid sequence of SEQ TD NO: 10.

Clause 91 The viral vector system of any one of clauses 78-90, wherein the second polypeptide domain is fused to the C-terminus, N-terminus, or both, of the first polypeptide domain.

Clause 92. The viral vector system of any one of clauses 78-91, further comprising a nuclear localization sequence.

Clause 93. The viral vector system of any one of clauses 78-92, further comprising a linker connecting the first polypeptide domain to the second polypeptide domain.

Clause 94. The viral vector system of any one of clauses 78-93, wherein the fusion protein comprises an amino acid sequence of SEQ ID NO. 13.

Clause 95. The viral vector system of any one of clauses 78-94, wherein the fusion protein is encoded by a polynucleotide sequence comprising a polynucleotide sequence of SEQ ID NO: 14.

Clause 96. The viral vector system of any one of clauses 78-95, wherein the viral vector is a lentiviral vector.

Clause 97. The viral vector system of any one of clauses 78-96, wherein the viral vector is an episomal integrase-deficient lentiviral vector (IDLV) or an episomal integrase-competent lentiviral vector (ICLV).

Clause 98. A method of reversing DNA damage in a subject suffering from a disease or disorder associated with elevated SNCA expression levels, the method comprising contacting the cell or subject with at least one of the composition of clauses 1-26, the isolated polynucleotide of clause 27, the vector of any one of clauses 28-31, the pharmaceutical composition of clause 33, or combinations thereof, in an amount sufficient to modulate expression of the gene.

Clause 99. A method of rescuing aging-related abnormal nuclei in a subject suffering from a disease or disorder associated with elevated SNCA expression levels, the method comprising contacting the cell or subject with at least one of the composition of clauses 1-26, the isolated polynucleotide of clause 27, the vector of any one of clauses 28-31, the pharmaceutical composition of clause 33, or combinations thereof, in an amount sufficient to modulate expression of the gene.

Clause 100. A method of increasing nuclear circularity or decreasing folded nuclei in a subject suffering from a disease or disorder associated with elevated SNCA expression levels, the method comprising contacting the cell or subject with at least one of the composition of clauses 1-26, the isolated polynucleotide of clause 27, the vector of any one of clauses 28-31, the pharmaceutical composition of clause 33, or combinations thereof, in an amount sufficient to modulate expression of the gene.

Clause 101. A method of reversing DNA damage in a subject suffering from a disease or disorder associated with elevated SNCA expression levels, the method comprising contacting the cell or subject with (a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b)(i) at least one guide RNA (gRNA) that targets the fusion molecule to a target region within the SNCA gene or (b)(ii) a nucleic acid sequence encoding at least one gRNA that targets the fusion protein to a target region within the SNCA gene, in an amount sufficient to modulate expression of the gene.

Clause 102. A method of rescuing aging-related abnormal nuclei in a subject suffering from a disease or disorder associated with elevated SNCA expression levels, the method comprising contacting the cell or subject with: (a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b)(i) at least one guide RNA (gRNA) that targets the fusion molecule to a target region within the SNCA gene or (b)(ii) a nucleic acid sequence encoding at least one gRNA that targets the fusion protein to a target region within the SNCA gene, in an amount sufficient to modulate expression of the gene.

Clause 103. A method of increasing nuclear circularity or decreasing folded nuclei in a subject suffering from a disease or disorder associated with elevated SNCA expression levels, the method comprising contacting the cell or subject with: (a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity, and (b)(i) at least one guide RNA (gRNA) that targets the fusion molecule to a target region within the SNCA gene or (b)(ii) a nucleic acid sequence encoding at least one gRNA that targets the fusion protein to a target region within the SNCA gene, in an amount sufficient to modulate expression of the gene.

Clause 104. The composition of any one of clauses 22-26, wherein the viral vector comprises a polynucleotide sequence of SEQ ID NO. 38, SEQ ID NO. 41, SEQ ID NO. 40, or SEQ ID NO: 39.

Clause 105. The vector of any one of clauses 28-31, wherein the viral vector comprises a polynucleotide sequence of SEQ ID NO: 38, SEQ ID NO: 41, SEQ ID NO: 40, or SEQ ID NO: 39.

Clause 106. The method of any one of clauses 59-62, wherein the viral vector comprises a polynucleotide sequence of SEQ ID NO: 38, SEQ ID NO: 41, SEQ ID NO: 40, or SEQ ID NO: 39.

Clause 107 The viral vector system of any one of clauses 78-97, wherein the viral vector comprises a polynucleotide sequence of SEQ ID NO: 38, SEQ ID NO: 41, SEQ ID NO: 40, or SEQ ID NO: 39.

Appendix (SEQUENCES)
Streptococcus pyogenes dCas amino acid sequence
(SEQ ID NO: 10)
MDKKYSIGLAIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLFEENPINASGVDAKA
ILSARLSKSRRLENLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIVDLLFKTNRKVT
VKQLKEDYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDA
IVPQSFLKDDSIDNKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEAKGYKEV
KKDLIIKLPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAAFKYFDTT
IDRKRYTSTKEVLDATLIHQSITGLYETRIDLSQLGGD
DNMT3A amino acid sequence
(SEQ ID NO: 11)
PSRLQMFFANNHDQEFDPPKVYPPVPAEKRKPIRVLSLFDGIATGLLVLKDLGIQVDRYIASEVCEDSIT
VGMVRHQGKIMYVGDVRSVTQKHIQEWGPFDLVIGGSPCNDLSIVNPARKGLYEGTGRLFFEFYRLLHDA
RPKEGDDRPFFWLFENVVAMGVSDKRDISRFLESNPVMIDAKEVSAAHRARYFWGNLPGMNRPLASTVND
KLELQECLEHGRIAKFSKVRTITTRSNSIKQGKDQHFPVFMNEKEDILWCTEMERVFGFPVHYTDVSNMS
RLARQRLLGRSWSVPVIRHLFAPLKEYFACV
DNMT3A nucleotide sequence
(SEQ ID NO: 12)
CCCTCCCGGCTCCAGATGttcttcgctaataaccacgaccaggaatttgaccctccaaaggtttacccac
ctgtcccagctgagaagaggaagcccatccgggtgctgtctctctttgatggaatcgctacagggctcct
ggtgctgaaggacttgggcattcaggtggaccgctacattgcctcggaggtgtgtgaggactccatcacg
gtgggcatggtgcggcaccaggggaagatcatgtacgtcggggacgtccgcagcgtcacacagaagcata
tccaggagtggggcccattcgatctggtgattgggggcagtccctgcaatgacctctccatcgtcaaccc
tgctcgcaagggcctctacgagggcactggccggctcttctttgagttctaccgcctcctgcatgatgcg
cggcccaaggagggagatgatcgccccttcttctggctctttgagaatgtggtggccatgggcgttagtg
acaagagggacatctcgcgatttctcgagtccaaccctgtgatgattgatgccaaagaagtgtcagctgc
acacagggcccgctacttctggggtaaccttcccggtatgaacaggccgttggcatccactgtgaatgat
aagctggagctgcaggagtgtctggagcatggcaggatagccaagttcagcaaagtgaggaccattacta
cgaggtcaaactccataaagcagggcaaaGACCAGCATTTTCCTGTGTTCATGAATGAGAAAGAGgacat
cttatggtgcactgaaatggaaagggtatttggtttcccagtccactatactgacgtgtccaacatgagc
cgcttggcgaggcagagactgctgggccggtcatggagcgtgccagtcatccgccacctcttcgctcCGC
TGAAGGAGTATTTTGCGTGTGTG
dCas9-DNMT3A fusion protein (aa sequence)
(SEQ ID NO: 13)
DKKYSIGLAIGTNSVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAKAI
LSARLSKSRRLENLIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDNGSIPHQIHLGELHA
ILRRQEDFYPFLKDNREKIEKILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQELDINRLSDYDVDAI
VPQSFLKDDSIDNKVLTRSDKNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKEVK
KDLIIKLPKYSLFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGAPAAFKYFDTTI
DRKRYTSTKEVLDATLIHQSITGLYETRIDLSQLGGDKRPAATKKAGQAKKKKLEGGGGSGSPSRIQMFF
ANNHDQEFDPPKVYPPVPAEKRKPIRVLSLFDGIATGLLVLKDLGIQVDRYIASEVCEDSITVGMVRHQG
KIMYVGDVRSVTQKHIQEWGPFDLVIGGSPCNDLSIVNPARKGLYEGTGRLFFEFYRLLHDARPKEGDDR
PFFWLFENVVAMGVSDKRDISRFLESNPVMIDAKEVSAAHRARYFWGNLPGMNRPLASTVNDKLELQECL
EHGRIAKFSKVRTITTRSNSIKQGKDQHFPVFMNEKEDILWCTEMERVFGFPVHYTDVSNMSRLARQRLL
GRSWSVPVIRHLFAPLKEYFAC
dCas9-DNMT3A fusion protein (nt sequence)
(SEQ ID NO: 14)
GACAAGAAGTACAGCATCGGCCTGGCCATCGGCACCAACTCTGTGGGCTGGGCCGTGATCACCGACGAGT
ACAAGGTGCCCAGCAAGAAATTCAAGGTGCTGGGCAACACCGACCGGCACAGCATCAAGAAGAACCTGAT
CGGAGCCCTGCTGTTCGACAGCGGCGAAACAGCCGAGGCCACCCGGCTGAAGAGAACCGCCAGAAGAAGA
TACACCAGACGGAAGAACCGGATCTGCTATCTGCAAGAGATCTTCAGCAACGAGATGGCCAAGGTGGACG
ACAGCTTCTTCCACAGACTGGAAGAGTCCTTCCTGGTGGAAGAGGATAAGAAGCACGAGCGGCACCCCAT
CTTCGGCAACATCGTGGACGAGGTGGCCTACCACGAGAAGTACCCCACCATCTACCACCTGAGAAAGAAA
CTGGTGGACAGCACCGACAAGGCCGACCTGCGGCTGATCTATCTGGCCCTGGCCCACATGATCAAGTTCC
GGGGCCACTTCCTGATCGAGGGCGACCTGAACCCCGACAACAGCGACGTGGACAAGCTGTTCATCCAGCT
GGTGCAGACCTACAACCAGCTGTTCGAGGAAAACCCCATCAACGCCAGCGGCGTGGACGCCAAGGCCATC
CTGTCTGCCAGACTGAGCAAGAGCAGACGGCTGGAAAATCTGATCGCCCAGCTGCCCGGCGAGAAGAAGA
ATGGCCTGTTCGGCAACCTGATTGCCCTGAGCCTGGGCCTGACCCCCAACTTCAAGAGCAACTTCGACCT
GGCCGAGGATGCCAAACTGCAGCTGAGCAAGGACACCTACGACGACGACCTGGACAACCTGCTGGCCCAG
ATCGGCGACCAGTACGCCGACCTGTTTCTGGCCGCCAAGAACCTGTCCGACGCCATCCTGCTGAGCGACA
TCCTGAGAGTGAACACCGAGATCACCAAGGCCCCCCTGAGCGCCTCTATGATCAAGAGATACGACGAGCA
CCACCAGGACCTGACCCTGCTGAAAGCTCTCGTGCGGCAGCAGCTGCCTGAGAAGTACAAAGAGATTTTC
TTCGACCAGAGCAAGAACGGCTACGCCGGCTACATTGACGGCGGAGCCAGCCAGGAAGAGTTCTACAAGT
TCATCAAGCCCATCCTGGAAAAGATGGACGGCACCGAGGAACTGCTCGTGAAGCTGAACAGAGAGGACCT
GCTGCGGAAGCAGCGGACCTTCGACAACGGCAGCATCCCCCACCAGATCCACCTGGGAGAGCTGCACGCC
ATTCTGCGGCGGCAGGAAGATTTTTACCCATTCCTGAAGGACAACCGGGAAAAGATCGAGAAGATCCTGA
CCTTCCGCATCCCCTACTACGTGGGCCCTCTGGCCAGGGGAAACAGCAGATTCGCCTGGATGACCAGAAA
GAGCGAGGAAACCATCACCCCCTGGAACTTCGAGGAAGTGGTGGACAAGGGCGCTTCCGCCCAGAGCTTC
ATCGAGCGGATGACCAACTTCGATAAGAACCTGCCCAACGAGAAGGTGCTGCCCAAGCACAGCCTGCTGT
ACGAGTACTTCACCGTGTATAACGAGCTGACCAAAGTGAAATACGTGACCGAGGGAATGAGAAAGCCCGC
CTTCCTGAGCCGCGAGCAGAAAAAGGCCATCGTGGACCTGCTGTTCAAGACCAACCGGAAAGTGACCGTG
AAGCAGCTGAAAGAGGACTACTTCAAGAAAATCGAGTGCTTCGACTCCGTGGAAATCTCCGGCGTGGAAG
ATCGGTTCAACGCCTCCCTGGGCACATACCACGATCTGCTGAAAATTATCAAGGACAAGGACTTCCTGGA
CAATGAGGAAAACGAGGACATTCTGGAAGATATCGTGCTGACCCTGACACTGTTTGAGGACAGAGAGATG
ATCGAGGAACGGCTGAAAACCTATGCCCACCTGTTCGACGACAAAGTGATGAAGCAGCTGAAGCGGCGGA
GATACACCGGCTGGGGCAGGCTGAGCCGGAAGCTGATCAACGGCATCCGGGACAAGCAGTCCGGCAAGAC
AATCCTGGATTTCCTGAAGTCCGACGGCTTCGCCAACAGAAACTTCATGCAGCTGATCCACGACGACAGC
CTGACCTTTAAAGAGGACATCCAGAAAGCCCAGGTGTCCGGCCAGGGCGATAGCCTGCACGAGCACATTG
CCAATCTGGCCGGCAGCCCCGCCATTAAGAAGGGCATCCTGCAGACAGTGAAGGTGGTGGACGAGCTCGT
GAAAGTGATGGGCCGGCACAAGCCCGAGAACATCGTGATCGAAATGGCCAGAGAGAACCAGACCACCCAG
AAGGGACAGAAGAACAGCCGCGAGAGAATGAAGCGGATCGAAGAGGGCATCAAAGAGCTGGGCAGCCAGA
TCCTGAAAGAACACCCCGTGGAAAACACCCAGCTGCAGAACGAGAAGCTGTACCTGTACTACCTGCAGAA
TGGGCGGGATATGTACGTGGACCAGGAACTGGACATCAACCGGCTGTCCGACTACGATGTGGACGCTATC
GTGCCTCAGAGCTTTCTGAAGGACGACTCCATCGACAACAAGGTGCTGACCAGAAGCGACAAGAACCGGG
GCAAGAGCGACAACGTGCCCTCCGAAGAGGTCGTGAAGAAGATGAAGAACTACTGGCGGCAGCTGCTGAA
CGCCAAGCTGATTACCCAGAGAAAGTTCGACAATCTGACCAAGGCCGAGAGAGGCGGCCTGAGCGAACTG
GATAAGGCCGGCTTCATCAAGAGACAGCTGGTGGAAACCCGGCAGATCACAAAGCACGTGGCACAGATCC
TGGACTCCCGGATGAACACTAAGTACGACGAGAATGACAAGCTGATCCGGGAAGTGAAAGTGATCACCCT
GAAGTCCAAGCTGGTGTCCGATTTCCGGAAGGATTTCCAGTTTTACAAAGTGCGCGAGATCAACAACTAC
CACCACGCCCACGACGCCTACCTGAACGCCGTCGTGGGAACCGCCCTGATCAAAAAGTACCCTAAGCTGG
AAAGCGAGTTCGTGTACGGCGACTACAAGGTGTACGACGTGCGGAAGATGATCGCCAAGAGCGAGCAGGA
AATCGGCAAGGCTACCGCCAAGTACTTCTTCTACAGCAACATCATGAACTTTTTCAAGACCGAGATTACC
CTGGCCAACGGCGAGATCCGGAAGCGGCCTCTGATCGAGACAAACGGCGAAACCGGGaAGATCGTGTGGG
ATAAGGGCCGGGATTTTGCCACCGTGCGGAAAGTGCTGAGCATGCCCCAAGTGAATATCGTGAAAAAGAC
CGAGGTGCAGACAGGCGGCTTCAGCAAAGAGTCTATCCTGCCCAAGAGGAACAGCGATAAGCTGATCGCC
AGAAAGAAGGACTGGGACCCTAAGAAGTACGGCGGCTTCGACAGCCCCACCGTGGCCTATTCTGTGCTGG
TGGTGGCCAAAGTGGAAAAGGGCAAGTCCAAGAAACTGAAGAGTGTGAAAGAGCTGCTGGGGATCACCAT
CATGGAAAGAAGCAGCTTCGAGAAGAATCCCATCGACTTTCTGGAAGCCAAGGGCTACAAAGAAGTGAAA
AAGGACCTGATCATCAAGCTGCCTAAGTACTCCCTGTTCGAGCTGGAAAACGGCCGGAAGAGAATGCTGG
CCTCTGCCGGCGAACTGCAGAAGGGAAACGAACTGGCCCTGCCCTCCAAATATGTGAACTTCCTGTACCT
GGCCAGCCACTATGAGAAGCTGAAGGGCTCCCCCGAGGATAATGAGCAGAAACAGCTGTTTGTGGAACAG
CACAAGCACTACCTGGACGAGATCATCGAGCAGATCAGCGAGTTCTCCAAGAGAGTGATCCTGGCCGACG
CTAATCTGGACAAAGTGCTGTCCGCCTACAACAAGCACCGGGATAAGCCCATCAGAGAGCAGGCCGAGAA
TATCATCCACCTGTTTACCCTGACCAATCTGGGAGCCCCTGCCGCCTTCAAGTACTTTGACACCACCATC
GACCGGAAGAGGTACACCAGCACCAAAGAGGTGCTGGACGCCACCCTGATCCACCAGAGCATCACCGGCC
TGTACGAGACACGGATCGACCTGTCTCAGCTGGGAGGCGACAAAAGGCCGGCGGCCACGAAAAAGGCCGG
ACAGGCCAAAAAGAAAAAGCTCGAGGGCGGAGGCGGGAGCGGATCCCCCTCCCGGCTCCAGATGttcttc
gctaataaccacgaccaggaatttgaccctccaaaggtttacccacctgtcccagctgagaagaggaagc
ccatccgggtgctgtctctctttgatggaatcgctacagggctcctggtgctgaaggacttgggcattca
ggtggaccgctacattgcctcggaggtgtgtgaggactccatcacggtgggcatggtgcggcaccagggg
aagatcatgtacgtcggggacgtccgcagcgtcacacagaagcatatccaggagtggggcccattcgatc
tggtgattgggggcagtccctgcaatgacctctccatcgtcaaccctgctcgcaagggcctctacgaggg
cactggccggctcttctttgagttctaccgcctcctgcatgatgcgcggcccaaggagggagatgatcgc
cccttcttctggctctttgagaatgtggtggccatgggcgttagtgacaagagggacatctcgcgatttc
tcgagtccaaccctgtgatgattgatgccaaagaagtgtcagctgcacacagggcccgctacttctgggg
taaccttcccggtatgaacaggccgttggcatccactgtgaatgataagctggagctgcaggagtgtctg
gagcatggcaggatagccaagttcagcaaagtgaggaccattactacgaggtcaaactccataaagcagg
gcaaaGACCAGCATTTTCCTGTGTTCATGAATGAGAAAGAGgacatcttatggtgcactgaaatggaaag
ggtatttggtttcccagtccactatactgacgtgtccaacatgagccgcttggcgaggcagagactgctg
ggccggtcatggagcgtgccagtcatccgccacctcttcgctcCGCTGAAGGAGTATTTTGCGTGTGTG
pBK500 (all-in-one lentiviral vector with gRNA4)- Lentivirus construct
sequence containing fusion protein and gRNA
(SEQ ID NO: 38)
gtcgacggatcgggagatctcccgatcccctatggtgcactctcagtacaatctgctctgatgccgcata
gttaagccagtatctgctccctgcttgtgtgttggaggtcgctgagtagtgcgcgagcaaaatttaagct
acaacaaggcaaggcttgaccgacaattgcatgaagaatctgcttagggttaggcgttttgcgctgcttc
gcgatgtacgggccagatatacgcgttgacattgattattgactagttattaatagtaatcaattacggg
gtcattagttcatagcccatatatggagttccgcgttacataacttacggtaaatggcccgcctggctga
ccgcccaacgacccccgcccattgacgtcaataatgacgtatgttcccatagtaacgccaatagggactt
tccattgacgtcaatgggtggagtatttacggtaaactgcccacttggcagtacatcaagtgtatcatat
gccaagtacgccccctattgacgtcaatgacggtaaatggcccgcctggcattatgcccagtacatgacc
ttatgggactttcctacttggcagtacatctacgtattagtcatcgctattaccatggtgatgcggtttt
ggcagtacatcaatgggcgtggatagcggtttgactcacggggatttccaagtctccaccccattgacgt
caatgggagtttgttttggcaccaaaatcaacgggactttccaaaatgtcgtaacaactccgccccattg
acgcaaatgggcggtaggcgtgtacggtgggaggtctatataagcagcgcgttttgcctgtactgggtct
ctctggttagaccagatctgagcctgggagctctctggctaactagggaacccactgcttaagcctcaat
aaagcttgccttgagtgcttcaagtagtgtgtgcccgtctgttgtgtgactctggtaactagagatccct
cagacccttttagtcagtgtggaaaatctctagcagtggcgcccgaacagggacttgaaagcgaaaggga
aaccagaggagctctctcgacgcaggactcggcttgctgaagcgcgcacggcaagaggcgaggggcggcg
actggtgagtacgccaaaaattttgactagcggaggctagaaggagagagatgggtgcgagagcgtcagt
attaagcgggggagaattagatcgcgatgggaaaaaattcggttaaggccagggggaaagaaaaaatata
aattaaaacatatagtatgggcaagcagggagctagaacgattcgcagttaatcctggcctgttagaaac
atcagaaggctgtagacaaatactgggacagctacaaccatcccttcagacaggatcagaagaacttaga
tcattatataatacagtagcaaccctctattgtgtgcatcaaaggatagagataaaagacaccaaggaag
ctttagacaagatagaggaagagcaaaacaaaagtaagaccaccgcacagcaagcggccgctgatcttca
gacctggaggaggagatatgagggacaattggagaagtgaattatataaatataaagtagtaaaaattga
accattaggagtagcacccaccaaggcaaagagaagagtggtgcagagagaaaaaagagcagtgggaata
ggagctttgttccttgggttcttgggagcagcaggaagcactatgggcgcagcgtcaatgacgctgacgg
tacaggccagacaattattgtctggtatagtgcagcagcagaacaatttgctgagggctattgaggcgca
acagcatctgttgcaactcacagtctggggcatcaagcagctccaggcaagaatcctggctgtggaaaga
tacctaaaggatcaacagctcctggggatttggggttgctctggaaaactcatttgcaccactgctgtgc
cttggaatgctagttggagtaataaatctctggaacagatttggaatcacacgacctggatggagtggga
cagagaaattaacaattacacaagattaatacactccttaattgaagaatcgcaaaaccagcaagaaaag
aatgaacaagaattattggaattagataaatgggcaagtttgtggaattggtttaacataacaaattggc
tgtggtatataaaattattcataatgatagtaggaggcttggtaggtttaagaatagtttttgctgtact
ttctatagtgaatagagttaggcagggatattcaccattatcgtttcagacccacctcccaaccccgagg
ggacccgacaggcccgaaggaatagaagaagaaggtggagagagagacagagacagatccattcgattag
tgaacggatcggcactgcgtgcgccaattctgcagacaaatggcagtattcatccacaattttaaaagaa
aaggggggattggggggtacagtgcaggggaaagaatagtagacataatagcaacagacatacaaactaa
agaattacaaaaacaaattacaaaaattcaaaattttcgggtttattacagggacagcagagatccagtt
tggTTAATTAATGGGCGGGACGTTAACGGGGCGGAACGGTACCgagggcctatttcccatgattccttca
tatttgcatatacgatacaaggctgttagagagataattagaattaatttgactgtaaacacaaagatat
tagtacaaaatacgtgacgtagaaagtaataatttcttgggtagtttgcagttttaaaattatgttttaa
aatggactatcatatgcttaccgtaacttgaaagtatttcgatttcttggctttatatatcttGTGGAAA
GGACGAAAcaccgCTGCTCAGGGTAGATAGCTGGTTTtagagctaGAAAtagcaagttaaaataaggcta
gtccgttatcaacttgaaaaagtggcaccgagtcggtgcTTTTTTgaattcgctagctaggtattgaaag
gagtgggaattggctccggtgcccgtcagtgggcagagcgcacatcgcccacagtccccgagaagttggg
gggaggggtcggcaattgatccggtgcctagagaaggtggcgcggggtaaactgggaaagtgatgtcgtg
tactggctccgcctttttcccgagggtgggggagaaccgtatataagtgcagtagtcgccgtgaacgttc
tttttcgcaacgggtttgccgccagaacacaggaccggttctagagcgctgccaccATGGACAAGAAGTA
CAGCATCGGCCTGGACATCGGCACCAACTCTGTGGGCTGGGCCGTGATCACCGACGAGTACAAGGTGCCC
AGCAAGAAATTCAAGGTGCTGGGCAACACCGACCGGCACAGCATCAAGAAGAACCTGATCGGAGCCCTGC
TGTTCGACAGCGGCGAAACAGCCGAGGCCACCCGGCTGAAGAGAACCGCCAGAAGAAGATACACCAGACG
GAAGAACCGGATCTGCTATCTGCAAGAGATCTTCAGCAACGAGATGGCCAAGGTGGACGACAGCTTCTTC
CACAGACTGGAAGAGTCCTTCCTGGTGGAAGAGGATAAGAAGCACGAGCGGCACCCCATCTTCGGCAACA
TCGTGGACGAGGTGGCCTACCACGAGAAGTACCCCACCATCTACCACCTGAGAAAGAAACTGGTGGACAG
CACCGACAAGGCCGACCTGCGGCTGATCTATCTGGCCCTGGCCCACATGATCAAGTTCCGGGGCCACTTC
CTGATCGAGGGCGACCTGAACCCCGACAACAGCGACGTGGACAAGCTGTTCATCCAGCTGGTGCAGACCT
ACAACCAGCTGTTCGAGGAAAACCCCATCAACGCCAGCGGCGTGGACGCCAAGGCCATCCTGTCTGCCAG
ACTGAGCAAGAGCAGACGGCTGGAAAATCTGATCGCCCAGCTGCCCGGCGAGAAGAAGAATGGCCTGTTC
GGAAACCTGATTGCCCTGAGCCTGGGCCTGACCCCCAACTTCAAGAGCAACTTCGACCTGGCCGAGGATG
CCAAACTGCAGCTGAGCAAGGACACCTACGACGACGACCTGGACAACCTGCTGGCCCAGATCGGCGACCA
GTACGCCGACCTGTTTCTGGCCGCCAAGAACCTGTCCGACGCCATCCTGCTGAGCGACATCCTGAGAGTG
AACACCGAGATCACCAAGGCCCCCCTGAGCGCCTCTATGATCAAGAGATACGACGAGCACCACCAGGACC
TGACCCTGCTGAAAGCTCTCGTGCGGCAGCAGCTGCCTGAGAAGTACAAAGAGATTTTCTTCGACCAGAG
CAAGAACGGCTACGCCGGCTACATTGACGGCGGAGCCAGCCAGGAAGAGTTCTACAAGTTCATCAAGCCC
ATCCTGGAAAAGATGGACGGCACCGAGGAACTGCTCGTGAAGCTGAACAGAGAGGACCTGCTGCGGAAGC
AGCGGACCTTCGACAACGGCAGCATCCCCCACCAGATCCACCTGGGAGAGCTGCACGCCATTCTGCGGCG
GCAGGAAGATTTTTACCCATTCCTGAAGGACAACCGGGAAAAGATCGAGAAGATCCTGACCTTCCGCATC
CCCTACTACGTGGGCCCTCTGGCCAGGGGAAACAGCAGATTCGCCTGGATGACCAGAAAGAGCGAGGAAA
CCATCACCCCCTGGAACTTCGAGGAAGTGGTGGACAAGGGCGCTTCCGCCCAGAGCTTCATCGAGCGGAT
GACCAACTTCGATAAGAACCTGCCCAACGAGAAGGTGCTGCCCAAGCACAGCCTGCTGTACGAGTACTTC
ACCGTGTATAACGAGCTGACCAAAGTGAAATACGTGACCGAGGGAATGAGAAAGCCCGCCTTCCTGAGCG
GCGAGCAGAAAAAGGCCATCGTGGACCTGCTGTTCAAGACCAACCGGAAAGTGACCGTGAAGCAGCTGAA
AGAGGACTACTTCAAGAAAATCGAGTGCTTCGACTCCGTGGAAATCTCCGGCGTGGAAGATCGGTTCAAC
GCCTCCCTGGGCACATACCACGATCTGCTGAAAATTATCAAGGACAAGGACTTCCTGGACAATGAGGAAA
ACGAGGACATTCTGGAAGATATCGTGCTGACCCTGACACTGTTTGAGGACAGAGAGATGATCGAGGAACG
GCTGAAAACCTATGCCCACCTGTTCGACGACAAAGTGATGAAGCAGCTGAAGCGGCGGAGATACACCGGC
TGGGGCAGGCTGAGCCGGAAGCTGATCAACGGCATCCGGGACAAGCAGTCCGGCAAGACAATCCTGGATT
TCCTGAAGTCCGACGGCTTCGCCAACAGAAACTTCATGCAGCTGATCCACGACGACAGCCTGACCTTTAA
AGAGGACATCCAGAAAGCCCAGGTGTCCGGCCAGGGCGATAGCCTGCACGAGCACATTGCCAATCTGGCC
GGCAGCCCCGCCATTAAGAAGGGCATCCTGCAGACAGTGAAGGTGGTGGACGAGCTCGTGAAAGTGATGG
GCCGGCACAAGCCCGAGAACATCGTGATCGAAATGGCCAGAGAGAACCAGACCACCCAGAAGGGACAGAA
GAACAGCCGCGAGAGAATGAAGCGGATCGAAGAGGGCATCAAAGAGCTGGGCAGCCAGATCCTGAAAGAA
CACCCCGTGGAAAACACCCAGCTGCAGAACGAGAAGCTGTACCTGTACTACCTGCAGAATGGGCGGGATA
TGTACGTGGACCAGGAACTGGACATCAACCGGCTGTCCGACTACGATGTGGACCATATCGTGCCTCAGAG
CTTTCTGAAGGACGACTCCATCGACAACAAGGTGCTGACCAGAAGCGACAAGAACCGGGGCAAGAGCGAC
AACGTGCCCTCCGAAGAGGTCGTGAAGAAGATGAAGAACTACTGGCGGCAGCTGCTGAACGCCAAGCTGA
TTACCCAGAGAAAGTTCGACAATCTGACCAAGGCCGAGAGAGGCGGCCTGAGCGAACTGGATAAGGCCGG
CTTCATCAAGAGACAGCTGGTGGAAACCCGGCAGATCACAAAGCACGTGGCACAGATCCTGGACTCCCGG
ATGAACACTAAGTACGACGAGAATGACAAGCTGATCCGGGAAGTGAAAGTGATCACCCTGAAGTCCAAGC
TGGTGTCCGATTTCCGGAAGGATTTCCAGTTTTACAAAGTGCGCGAGATCAACAACTACCACCACGCCCA
CGACGCCTACCTGAACGCCGTCGTGGGAACCGCCCTGATCAAAAAGTACCCTAAGCTGGAAAGCGAGTTC
GTGTACGGCGACTACAAGGTGTACGACGTGCGGAAGATGATCGCCAAGAGCGAGCAGGAAATCGGCAAGG
CTACCGCCAAGTACTTCTTCTACAGCAACATCATGAACTTTTTCAAGACCGAGATTACCCTGGCCAACGG
CGAGATCCGGAAGCGGCCTCTGATCGAGACAAACGGCGAAACCGGGGAGATCGTGTGGGATAAGGGCCGG
GATTTTGCCACCGTGCGGAAAGTGCTGAGCATGCCCCAAGTGAATATCGTGAAAAAGACCGAGGTGCAGA
CAGGCGGCTTCAGCAAAGAGTCTATCCTGCCCAAGAGGAACAGCGATAAGCTGATCGCCAGAAAGAAGGA
CTGGGACCCTAAGAAGTACGGCGGCTTCGACAGCCCCACCGTGGCCTATTCTGTGCTGGTGGTGGCCAAA
GTGGAAAAGGGCAAGTCCAAGAAACTGAAGAGTGTGAAAGAGCTGCTGGGGATCACCATCATGGAAAGAA
GCAGCTTCGAGAAGAATCCCATCGACTTTCTGGAAGCCAAGGGCTACAAAGAAGTGAAAAAGGACCTGAT
CATCAAGCTGCCTAAGTACTCCCTGTTCGAGCTGGAAAACGGCCGGAAGAGAATGCTGGCCTCTGCCGGC
GAACTGCAGAAGGGAAACGAACTGGCCCTGCCCTCCAAATATGTGAACTTCCTGTACCTGGCCAGCCACT
ATGAGAAGCTGAAGGGCTCCCCCGAGGATAATGAGCAGAAACAGCTGTTTGTGGAACAGCACAAGCACTA
CCTGGACGAGATCATCGAGCAGATCAGCGAGTTCTCCAAGAGAGTGATCCTGGCCGACGCTAATCTGGAC
AAAGTGCTGTCCGCCTACAACAAGCACCGGGATAAGCCCATCAGAGAGCAGGCCGAGAATATCATCCACC
TGTTTACCCTGACCAATCTGGGAGCCCCTGCCGCCTTCAAGTACTTTGACACCACCATCGACCGGAAGAG
GTACACCAGCACCAAAGAGGTGCTGGACGCCACCCTGATCCACCAGAGCATCACCGGCCTGTACGAGACA
CGGATCGACCTGTCTCAGCTGGGAGGCGACAAGCGACCTGCCGCCACAAAGAAGGCTGGACAGGCTAAGA
AGAAGAAAGATTACAAAGACGATGACGATAAGGGATCCGGCGCAACAAACTTCTCTCTGCTGAAACAAGC
CGGAGATGTCGAAGAGAATCCTGGACCGACCGAGTACAAGCCCACGGTGCGCCTCGCCACCCGCGACGAC
GTCCCCAGGGCCGTACGCACCCTCGCCGCCGCGTTCGCCGACTACCCCGCCACGCGCCACACCGTCGATC
CGGACCGCCACATCGAGCGGGTCACCGAGCTGCAAGAACTCTTCCTCACGCGCGTCGGGCTCGACATCGG
CAAGGTGTGGGTCGCGGACGACGGCGCCGCGGTGGCGGTCTGGACCACGCCGGAGAGCGTCGAAGCGGGG
GCGGTGTTCGCCGAGATCGGCCCGCGCATGGCCGAGTTGAGCGGTTCCCGGCTGGCCGCGCAGCAACAGA
TGGAAGGCCTCCTGGCGCCGCACCGGCCCAAGGAGCCCGCGTGGTTCCTGGCCACCGTCGGAGTCTCGCC
CGACCACCAGGGCAAGGGTCTGGGCAGCGCCGTCGTGCTCCCCGGAGTGGAGGCGGCCGAGCGCGCCGGG
GTGCCCGCCTTCCTGGAGACCTCCGCGCCCCGCAACCTCCCCTTCTACGAGCGGCTCGGCTTCACCGTCA
CCGCCGACGTCGAGGTGCCCGAAGGACCGCGCACCTGGTGCATGACCCGCAAGCCCGGTGCCTGAACGCG
TTAAGTCGACAATCAACCTCTGGATTACAAAATTTGTGAAAGATTGACTGGTATTCTTAACTATGTTGCT
CCTTTTACGCTATGTGGATACGCTGCTTTAATGCCTTTGTATCATGCTATTGCTTCCCGTATGGCTTTCA
TTTTCTCCTCCTTGTATAAATCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCATTGCCACCACCTGTCAGCTC
CTTTCCGGGACTTTCGCTTTCCCCCTCCCTATTGCCACGGCGGAACTCATCGCCGCCTGCCTTGCCCGCT
GCTGGACAGGGGCTCGGCTGTTGGGCACTGACAATTCCGTGGTGTTGTCGGGGAAATCATCGTCCTTTCC
TTGGCTGCTCGCCTGTGTTGCCACCTGGATTCTGCGCGGGACGTCCTTCTGCTACGTCCCTTCGGCCCTC
AATCCAGCGGACCTTCCTTCCCGCGGCCTGCTGCCGGCTCTGCGGCCTCTTCCGCGTCTTCGCCTTCGCC
CTCAGACGAGTCGGATCTCCCTTTGGGCCGCCTCCCCGCGTCGACTTTAAGACCAATGACTTACAAGGCA
GCTGTAGATCTTAGCCACTTTTTAAAAGAAAAGGGGGGACTGGAAGGGCTAATTCACTCCCAACGAAGAC
AAGATCTGCTTTTTGCTTGTACTGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTCAAGTAGTGTGTGCCCGTCTG
TTGTGTGACTCTGGTAACTAGAGATCCCTCAGACCCTTTTAGTCAGTGTGGAAAATCTCTAGCAGggccc
gtttaaacccgctgatcagcctcgactgtgccttctagttgccagccatctgttgtttgcccctcccccg
tgccttccttgaccctggaaggtgccactcccactgtcctttcctaataaaatgaggaaattgcatcgca
ttgtccgagtaggtgtcattctattctggggggtggggtggggcaggacagcaagggggaggattgggaa
gacaatagcaggcatgctggggatgcggtgggctctatggcttctgaggcggaaagaaccagctggggct
ctagggggtabacccacgcgccctgtagcggcgcattaagagcggcgggtgtggtggttacgcgcagagt
gaccgctacacttgccagcgccctagcgcccgctcctttcgctttcttcccttcctttctcgccacgttc
gccggctttccccgtcaagctctaaatcgggggctccctttagggttccgatttagtgctttacggcacc
tcgacaccaaaaaacttgattagggtgatggttcacgtagtgggccatcgccatgatagacggtttttcg
ccctttgacgttggagtccacgttctttaatagtggactcttgttccaaactggaacaacactcaaccct
atctcggtctattcttttgatttataagggattttgccgatttcggcctattggttaaaaaatgagctga
tttaacaaaaatttaacgcgaattaattctgtggaatgtgtgtcagttagggtgtggaaagtccccaggc
tccccagcaggcagaagtatgcaaagcatgcatctcaattagtcagcaaccaggtgtggaaagtccccag
gatccccagcaggcagaagtatgcaaagcatgcatctcaattagtcagcaaccatagtcccgcccataac
tccgcccatcccgcccctaactccgcccagttccgcccattctccgccccatggctgactaatttttttt
atttatgcagaggccgaggccgcctctgcctctgagctattccagaagtagtgaggaggcttttttggag
gactaggcctttgcaaaaagctccagggagattgtatatccattttcggatccgatcagcacgtgttgac
aattaatcatcggcatagtatatcggcatagtataatacgacaaggtgaggaactaaaccatggccaagt
tgaccagtgccgttccggtgctcaccgcgcgcgacgtcgccggagcggtcgagttctggaccgaccggct
cgggttctcccgggacttcgtggaggacgacttcgccggtgtggtccgggacgacgtgaccctgttcatc
agcgcggtccaggaccaggtggtgccggacaacaccctggcctgggtgtgggtgcgcggcctggacgagc
tgtacgccgagtggtcggaggtcgtgtccacgaacttccgggacgcctccgggccggccatgaccgagat
cggcgagcagccgtgggggcgggagttcgccctgcgcgacccggccggcaactgcgtgcacttcgtggcc
gaggagcaggactgacacgtgctacgagatttcgattccaccgccgccttctatgaaaggttgggcttcg
gaatcgttttccgggacgccggctggatgatcctccagcgaggggatctcatgctggagtbattcgcaca
ccccaacttgtttattgcagcttataatggttacaaataaagcaatagcatcacaaatttcacaaataaa
gcatttttttcactgcattctagttgtggtttgtccaaactcatcaatgtatcttatcatgtctgtatac
cgtcgacctctagctagagcttggcgtaatcatggtcatagctgtttcctgtgtgaaattgttatccgct
cacaattccacacaacatacgagccggaagcataaagtgtaaagcctggggtgcctaatgagtgagctaa
ctcacattaattgcgttgcgctcactgcccgctttccagtcgggaaacctgtcgtgccagctgcattaat
gaatcggccaacgcgcggggagaggcggtttgcgtattgggcgctcttccgcttcctcgctcactgactc
gctgcgctcggtcgttcggctgcggcgagcggtatcagctcactcaaaggcggtaatacggttatccaca
gaatcaggggataacgcaggaaagaacatgtgagcaaaaggccagcaaaaggccaggaaccgtaaaaagg
ccgcgttgctggcgtttttccataggctccgcccccctgacgagcatcacaaaaatcgacgctcaagtca
gaggtggcgaaacccgacaggactataaagataccaggcgtttccccctggaagctccctcgtgcgctct
cctgttccgaccctgccgcttaccggatacctgtccgcctttctcccttcgggaagcgtggcgctttctc
atagctcacgctgtaggtatctcagttcggtgtaggtcgttcgctccaagctgggctgtgtgcacgaacc
ccccgttcagcccgaccgctgcgccttatccggtaactatcgtcttgagtccaacccggtaagacacgac
ttatcgccactggcagcagccactggtaacaggattagcagagcgaggtatgtaggcggtgctacagagt
tcttgaagtggtggcctaactacggctacactagaagaacagtatttggtatctgcgctctgctgaagcc
agttaccttcggaaaaagagttggtagctcttgatccggcaaacaaaccaccgctggtagcggtggtttt
tttgtttgcaagcagcagattacgcgcagaaaaaaaggatctcaagaagatcctttgatcttttctacgg
ggtctgacgctcagtggaacgaaaactcacgttaagggattttggtcatgagattatcaaaaaggatctt
cacctagatccttttaaattaaaaatgaagttttaaatcaatctaaagtatatatgagtaaacttggtct
gacagttaccaatgcttaatcagtgaggcacctatctcagcgatctgtctatttcgttcatccatagttg
cctgactccccgtcgtgtagataactacgatacgggagggcttaccatctggccccagtgctgcaatgat
accgcgagacccacgctcaccggctccagatttatcagcaataaaccagccagccggaagggccgagcgc
agaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtt
tggtatggcttcattcagctccggttcccaacgatcaaggcgagttacatgatcccccatgttgtgcaaa
aaagcggttagctccttcggtcctccgatcgttgtcagaagtaagttggccgcagtgttatcactcatgg
ttatggcagcactgcataattctcttactgtcatgccatccgtaagatgcttttctgtgactggtgagta
ctcaaccaagtcattctgagaatagtgtatgcggcgaccgagttgctcttgcccggcgtcaatacgggat
aataccgcgccacatagcagaactttaaaagtgctcatcattggaaaacgttcttcggggcgaaaactct
caaggatcttaccgctgttgagatccagttcgatgtaacccactcgtgcacccaactgatcttcagcatc
ttttactttcaccagcgtttctgggtgagcaaaaacaggaaggcaaaatgccgcaaaaaagggaataagg
gcgacacggaaatgttgaatactcatactcttcctttttcaatattattgaagcatttatcagggttatt
gtctcatgagcggatacatatttgaatgtatttagaaaaataaacaaataggggttccgcgcacatttcc
ccgaaaagtgccacctgac
pBK546 complete sequence, plasmid carried dCas9-DNMT3A fused transgene
linked to puromycin selection gene via p2A cleavage signal (formerly
known as pBK492 vector (naïve (no gRNA-vector) - contains a catalytic
domain of DNMT3A fused to dCas9)
(SEQ ID NO: 39)
gtcgacggatcgggagatctcccgatcccctatggtgcactctcagtacaatctgctctgatgccgcata
gttaagccagtatctgctccctgcttgtgtgttggaggtcgctgagtagtgcgcgagcaaaatttaagct
acaacaaggcaaggcttgaccgacaattgcatgaagaatctgcttagggttaggcgttttgcgctgcttc
gcgatgtacgggccagatatacgcgttgacattgattattgactagttattaatagtaatcaattacggg
gtcattagttcatagcccatatatggagttccgcgttacataacttacggtaaatggcccgcctggctga
ccgcccaacgacccccgcccattgacgtcaataatgacgtatgttcccatagtaacgccaatagggactt
tccattgacgtcaatgggtggagtatttacggtaaactgcccacttggcagtacatcaagtgtatcatat
gccaagtacgccccctattgacgtcaatgacggtaaatggcccgcctggcattatgcccagtacatgacc
ttatgggactttcctacttggcagtacatctacgtattagtcatcgctattaccatggtgatgcggtttt
ggcagtacatcaatgggcgtggatagcggtttgactcacggggatttccaagtctccaccccattgacgt
caatgggagtttgttttggcaccaaaatcaacgggactttccaaaatgtcgtaacaactccgccccattg
acgcaaatgggcggtaggcgtgtacggtgggaggtctatataagcagcgcgttttgcctgtactgggtct
ctctggttagaccagatctgagcctgggagctctctggctaactagggaacccactgcttaagcctcaat
aaagcttgccttgagtgcttcaagtagtgtgtgcccgtctgttgtgtgactctggtaactagagatccct
cagacccttttagtcagtgtggaaaatctctagcagtggcgcccgaacagggacttgaaagcgaaaggga
aaccagaggagctctctcgacgcaggactcggcttgctgaagcgcgcacggcaagaggcgaggggcggcg
actggtgagtacgccaaaaattttgactagcggaggctagaaggagagagatgggtgcgagagcgtcagt
attaagcgggggagaattagatcgcgatgggaaaaaattcggttaaggccagggggaaagaaaaaatata
aattaaaacatatagtatgggcaagcagggagctagaacgattcgcagttaatcctggcctgttagaaac
atcagaaggctgtagacaaatactgggacagctacaaccatcccttcagacaggatcagaagaacttaga
tcattatataatacagtagcaaccctctattgtgtgcatcaaaggatagagataaaagacaccaaggaag
ctttagacaagatagaggaagagcaaaacaaaagtaagaccaccgcacagcaagcggccgctgatcttca
gacctggaggaggagatatgagggacaattggagaagtgaattatataaatataaagtagtaaaaattga
accattaggagtagcacccaccaaggcaaagagaagagtggtgcagagagaaaaaagagcagtgggaata
ggagctttgttccttgggttcttgggagcagcaggaagcactatgggcgcagcgtcaatgacgctgacgg
tacaggccagacaattattgtctggtatagtgcagcagcagaacaatttgctgagggctattgaggcgca
acagcatctgttgcaactcacagtctggggcatcaagcagctccaggcaagaatcctggctgtggaaaga
tacctaaaggatcaacagctcctggggatttggggttgctctggaaaactcatttgcaccactgctgtgc
cttggaatgctagttggagtaataaatctctggaacagatttggaatcacacgacctggatggagtggga
cagagaaattaacaattacacaagcttaatacactccttaattgaagaatcgcaaaaccagcaagaaaag
aatgaacaagaattattggaattagataaatgggcaagtttgtggaattggtttaacataacaaattggc
tgtggtatataaaattattcataatgatagtaggaggcttggtaggtttaagaatagtttttgctgtact
ttctatagtgaatagagttaggcagggatattcaccattatcgtttcagacccacctcccaaccccgagg
ggacccgacaggcccgaaggaatagaagaagaaggtggagagagagacagagacagatccattcgattag
tgaacggatcggcactgcgtgcgccaattctgcagacaaatggcagtattcatccacaattttaaaagaa
aaggggggattggggggtacagtgcaggggaaagaatagtagacataatagcaacagacatacaaactaa
agaattacaaaaacaaattacaaaaattcaaaattttcgggtttattacagggacagcagagatccagtt
tggTTAATTAATGGGCGGGACGTTAACGGGGCGGAACGGTACCgagggcctatttcccatgattccttca
tatttgcatatacgatacaaggctgttagagagataattagaattaatttgactgtaaacacaaagatat
tagtacaaaatacgtgacgtagaaagtaataatttcttgggtagtttgcagttttaaaattatgttttaa
aatggactatcatatgcttaccgtaacttgaaagtatttcgatttcttggctttatatatcttGTGGAAA
GGACGAAAcaccggagacgtgtacacgtctctgTTTtagagctaGAAAtagcaagttaaaataaggctag
tccgttatcaacttgaaaaagtggcaccgagtcggtgcTTTTTTgaattcgctagctaggtcttgaaagg
agtgggaattggctccggtgcccgtcagtgggcagagcgcacatcgcccacagtccccgagaagttgggg
ggaggggtcggcaattgatccggtgcctagagaaggtggcgcggggtaaactgggaaagtgatgtcgtgt
actggctccgcctttttcccgagggtgggggagaaccgtatataagtgcagtagtcgccgtgaacgttct
ttttcgcaacgggtttgccgccagaacacaggaccggtgccaccATGGACTATAAGGACCACGACGGAGA
CTACAAGGATCATGATATTGATTACAAAGACGATGACGATAAGATGGCCCCAAAGAAGAAGCGGAAGGTC
GGTATCCACGGAGTCCCAGCAGCCGACAAGAAGTACAGCATCGGCCTGGCCATCGGCACCAACTCTGTGG
GCTGGGCCGTGATCACCGACGAGTACAAGGTGCCCAGCAAGAAATTCAAGGTGCTGGGCAACACCGACCG
GCACAGCATCAAGAAGAACCTGATCGGAGCCCTGCTGTTCGACAGCGGCGAAACAGCCGAGGCCACCCGG
CTGAAGAGAACCGCCAGAAGAAGATACACCAGACGGAAGAACCGGATCTGCTATCTGCAAGAGATCTTCA
GCAACGAGATGGCCAAGGTGGACGACAGCTTCTTCCACAGACTGGAAGAGTCCTTCCTGGTGGAAGAGGA
TAAGAAGCACGAGCGGCACCCCATCTTCGGCAACATCGTGGACGAGGTGGCCTACCACGAGAAGTACCCC
ACCATCTACCACCTGAGAAAGAAACTGGTGGACAGCACCGACAAGGCCGACCTGCGGCTGATCTATCTGG
CCCTGGCCCACATGATCAAGTTCCGGGGCCACTTCCTGATCGAGGGCGACCTGAACCCCGACAACAGCGA
CGTGGACAAGCTGTTCATCCAGCTGGTGCAGACCTACAACCAGCTGTTCGAGGAAAACCCCATCAACGCC
AGCGGCGTGGACGCCAAGGCCATCCTGTCTGCCAGACTGAGCAAGAGCAGACGGCTGGAAAATCTGATCG
CCCAGCTGCCCGGCGAGAAGAAGAATGGCcTGTTCGGCAACCTGATTGCCCTGAGCCTGGGCCTGACCCC
CAACTTCAAGAGCAACTTCGACCTGGCCGAGGATGCCAAACTGCAGCTGAGCAAGGACACCTACGACGAC
GACCTGGACAACCTGCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCCCCCCTGAGCGCCTC
TATGATCAAGAGATACGACGAGCACCACCAGGACCTGACCCTGCTGAAAGCTCTCGTGCGGCAGCAGCTG
CCTGAGAAGTACAAAGAGATTTTCTTCGACCAGAGCAAGAACGGCTACGCCGGCTACATTGACGGCGGAG
CCAGCCAGGAAGAGTTCTACAAGTTCATCAAGCCCATCCTGGAAAAGATGGACGGCACCGAGGAACTGCT
CGTGAAGCTGAACAGAGAGGACCTGCTGCGGAAGCAGCGGACCTTCGACAACGGCAGCATCCCCCACCAG
ATCCACCTGGaAGAGCTGCACGCCATTCTGCGGCGGCAGGAAGATTTTTACCCATTCCTGAAGaACAACC
GGGAAAAGATCGAGAAGATCCTGACCTTCCGCATCCCCTACTACGTGGGCCCTCTGGCCAGGGGAAACAG
CAGATTCGCCTGGATGACCAGAAAGAGCGAGGAAACCATCACCCCCTGGAACTTCGAGGAAGTGGTGGAC
AAGGGCGCTTCCGCCCAGAGCTTCATCGAGCGGATGACCAACTTCGATAAGAACCTGCCCAACGAGAAGG
TGCTGCCCAAGCACAGCCTGCTGTACGAGTACTTCACCGTGTATAACGAGCTGACCAAAGTGAAATACGT
GACCGAGGGAATGAGAAAGCCCGCCTTCCTGAGCGGCGAGCAGAAAAAGGCCATCGTGGACCTGCTGTTC
AAGACCAACCGGAAAGTGACCGTGAAGCAGCTGAAAGAGGACTACTTCAAGAAAATCGAGTGCTTCGACT
CCGTGGAAATCTCCGGCGTGGAAGATCGGTTCAACGCCTCCCTGGGCACATACCACGATCTGCTGAAAAT
TATCAAGGACAAGGACTTCCTGGACAATGAGGAAAACGAGGACATTCTGGAAGATATCGTGCTGACCCTG
ACACTGTTTGAGGACAGAGAGATGATCGAGGAACGGCTGAAAACCTATGCCCACCTGTTCGACGACAAAG
TGATCAACCACCTGAACCGCCGGAGATACACCGGCTGGGGCAGGCTGACCCGGAAGCTGATCAACGGCAT
CCGGGACAAGCAGTCCGGCAAGACAATCCTGGATTTCCTGAAGTCCGACGGCTTCGCCAACAGAAACTTC
ATGCAGCTGATCCACGACGACAGCCTGACCTTTAAAGAGGACATCCAGAAAGCCCAGGTGTCCGGCCAGG
GCGATAGCCTCCACGACCACATTGCCAATCTGGCCGGCAGCCCCGCCATTAAGAAGGCCATCCTGCAGAC
AGTGAAGGTGGTGGACGAGCTCGTGAAAGTGATGGGCCGGCACAAGCCCGAGAACATCGTGATCGAAATG
GCCAGAGAGAACCAGACCACCCAGAAGGGACAGAAGAACAGCCGCGAGAGAATGAAGCGGATCGAAGAGG
GCATCAAAGAGCTGGGCAGCCAGATCCTGAAAGAACACCCCGTGGAAAACACCCAGCTGCAGAACGAGAA
GCTGTACCTGTACTACCTGCAGAATGGGCGGGATATGTACGTGGACCAGGAACTGGACATCAACCGGCTG
TCCGACTACGATGTGGACGCTATCGTGCCTCAGAGCTTTCTGAAGGACGACTCCATCGACAACAAGGTGC
TGACCAGAAGCGACAAGAACCGGGGCAAGAGCGACAACGTGCCCTCCGAAGAGGTCGTGAAGAAGATGAA
GAACTACTGGCGGCAGCTGCTGAACGCCAAGCTGATTACCCAGAGAAAGTTCGACAATCTGACCAAGGCC
GAGAGAGGCGGCCTGAGCGAACTGGATAAGGCCGGCTTCATCAAaAGACAGCTGGIGGAAACCCGGCAGA
TCACAAAGCACGTGGCACAGATCCTGGACTCCCGGATGAACACTAAGTACGACGAGANTGACAAGCTGAT
CCGGCAACTGAAAGTGATCACCCTGAAGTCCAAGCTGGTGTCCGATTTCCGCAAGGATTTCCAGTTTTAC
AAAGTGCGCGAGATCAACAACTACCACCACGCCCACGACGCCTACCTGAACGCCGTCGTGGGAACCGCCC
TGATCAAAAAGTACCCTAAGCTGGAAAGCGAGTTCGTGTACGGCGACTACAAGGTGTACGACGTGCGGAA
GATGATCGCCAAGAGCGAGCAGGAAATCGGCAAGGCTACCGCCAAGTACTTCTTCTACAGCAACATCATG
AACTTTTTCAAGACCGAGATTACCCTGGCCAACGGCGAGATCCGGAAGCGGCCTCTGATCGAGACAAACG
GCGAAACCGGGGAGATCGTGTGGGATAAGGGCCGGGATTTTGCCACCGTGCGGAAAGTGCTGAGCATGCC
CCAAGTGAATATCGTGAAAAAGAECGAGGTGCAGACAGGCGGCTTCAGCAAAGAGTCTATCCTGCCCAAG
AGGAACAGCGATAAGCTGATCGCCAGAAAGAAGGACTGGGACCCTAAGAAGTACGGCGGCTTCGACAGCC
CCACCGTGGCCTATTCTGTGCTGGTGGTGGCCAAAGTGGAAAAGGGCAAGTCCAAGAAACTGAAGAGTGT
GAAAGAGCTGCTGGGGATCACCATCATGGAAAGAAGCAGCTTCGAGAAGAATCCCATCGACTTTCTGGAA
GCCAAGGGCTACAAAGAAGTGAAAAAGGACCTGATCATCAAGCTGCCTAAGTACTCCCTGTTCGAGCTGG
AAAACGGCCGGAAGAGAATGCTGGCCTCTGCCGGCGAACTGCAGAAGGGAAACGAACTGGCCCTGCCCTC
CAAATATGTGAACTTCCTGTACCTGGCCAGCCACTATGAGAAGCTGAAGGGCTCCCCCGAGGATAATGAG
CAGAAACAGCTGTTTGTGGAACAGCACAAGCACTACCTGGACGAGATCATCGAGCAGATCAGCGAGTTCT
CCAAGAGAGTGATCCTGGCCGACGCTAATCTGGACAAAGTGCTGTCCGCCTACAACAAGCACCGGGATAA
GCCCATCAGAGAGCAGGCCGAGAATATCATCCACCTGTTTACCCTGACCAATCTGGGAGCCCCTGCCGCC
TTCAAGTACTTTGACACCACCATCGACCGGAAGAGGTACACCAGCACCAAAGAGGTGCTGGACGCCACCC
TGATCCACCAGAGCATCACCGGCCTGTACGAGACACGGATCGACCTGTCTCAGCTGGGAGGCGACAAAAG
GCCGGCGGCCACGAAAAAGGCCGGACAGGCCAAAAAGAAAAAGCTCGAGGGCGGAGGCGGGAGCGGATCC
CCCTCCCGGCTCCAGATGttcttcgctaataaccacgaccaggaatttgaccctccaaaggtttacccac
ctgtcccagctgagaagaggaagcccatccgggtgctgtctctctttgatggaatcgctacagggctcct
ggtgctgaaggacttgggcattcaggtggaccgctacattgcctcggaggtgtgtgaggactccatcacg
gtgggcatggtgcggcaccaggggaagatcatgtacgtcggggacgtccgcagcgtcacacagaagcata
tccaggagtggggcccattcgatctggtgattgggggcagtccctgcaatgacctctccatcgtcaaccc
tgctcgcaagggcctctacgagggcactggccggctcttctttgagttctaccgcctcctgcatgatgcg
cggcccaaggagggagatgatcgccccttcttctggctctttgagaatgtggtggccatgggcgttagtg
acaagagggacatctcgcgatttctcgagtccaaccctgtgatgattgatgccaaagaagtgtcagctgc
acacagggcccgctacttctggggtaaccttcccggtatgaacaggccgttggcatccactgtgaatgat
aagctggagctgcaggagtgtctggagcatggcaggatagccaagttcagcaaagtgaggaccattacta
cgaggtcaaactccataaagcagggcaaaGACCAGCATTTTCCTGTGTTCATGAATGAGAAAGAGgacat
cttatggtgcactgaaatggaaagggtatttggtttcccagtccactatactgacgtctccaacatgagc
cgcttggcgaggcagagactgctgggccggtcatggagcgtgccagtcatccgccacctcttcgctccgc
tgaagGAGTATTTTGCGTGTGTGTCCGGCCGGCCcGgatccGGCGCAACAAACTTCTCTCTGCTGAAACA
AGCCGGAGATGTCGAAGAGAATCCTGGACCGACCGAGTACAAGCCCACGGTGCGCCTCGCCACCCGCGAC
GACGTCCCCAGGGCCGTACGCACCCTCGCCGCCGCGTTCGCCGACTACCCCGCCACGCGCCACACCGTCG
ATCCGGACCGCCACATCGAGCGGGTCACCGAGCTGCAAGAACTCTTCCTCACGCGCGTCGGGCTCGACAT
CGGCAAGGTGTGGGTCGCGGACGACGGCGCCGCGGTGGCGGTCTGGACCACGCCGGAGAGCGTCGAAGCG
GGGGCGGTGTTCGCCGAGATCGGCCCGCGCATGGCCGAGTTGAGCGGTTCCCGGCTGGCCGCGCAGCAAC
AGATGGAAGGCCTCCTGGCGCCGCACCGGCCCAAGGAGCCCGCGTGGTTCCTGGCCACCGTCGGAGTCTC
GCCCGACCACCAGGGCAAGGGTCTGGGCAGCGCCGTCGTGCTCCCCGGAGTGGAGGCGGCCGAGCGCGCC
GGGGTGCCCGCCTTCCTGGAGACCTCCGCGCCCCGCAACCTCCCCTTCTACGAGCGGCTCGGCTTCACCG
TCACCGCCGACGTCGAGGTGCCCGAAGGACCGCGCACCTGGTGCATGACCCGCAAGCCCGGTGCCTGAAC
GCGTTAAGTCGACAATCAACCTCTGGATTACAAAATTTGTGAAAGATTGACTGGTATTCTTAACTATGTT
GCTCCTTTTACGCTATGTGGATACGCTGCTTTAATGCCTTTGTATCATGCTATTGCTTCCCGTATGGCTT
TCATTTTCTCCTCCTTGTATAAATCCTGGTTGCTGTCTCTTTATGAGGAGTTGTGGCCCGTTGTCAGGCA
ACGTGGCGTGGTGTGCACTGTGTTTGCTGACGCAACCCCCACTGGTTGGGGCATTGCCACCACCTGTCAG
CTCCTTTCCGGGACTTTCGCTTTCCCCCTCCCTATTGCCACGGCGGAACTCATCGCCGCCTGCCTTGCCC
GCTGCTGGACAGGGGCTCGGCTGTTGGGCACTGACAATTCCGTGGTGTTGTCGGGGAAATCATCGTCCTT
TCCTTGGCTGCTCGCCTGTGTTGCCACCTGGATTCTGCGCGGGACGTCCTTCTGCTACGTCCCTTCGGCC
CTCAATCCAGCGGACCTTCCTTCCCGCGGCCTGCTGCCGGCTCTGCGGCCTCTTCCGCGTCTTCGCCITC
GCCCTCAGACGAGTCGGATCTCCCTTTGGGCCGCCTCCCCGCGTCGACTTTAAGACCAATGACTTACAAG
GCAGCTGTAGATCTTAGCCACTTTTTAAAAGAAAAGGGGGGACTGGAAGGGCTAATTCACTCCCAACGAA
GACAAGATCTGCTTTTTGCTTGTACTGGGTCTCTCTGGTTAGACCAGATCTGAGCCTGGGAGCTCTCTGG
CTAACTAGGGAACCCACTGCTTAAGCCTCAATAAAGCTTGCCTTGAGTGCTTCAAGTAGTGTGTGCCCGT
CTGTTGTGTGACTCTGGTAACTAGAGATCCCTCAGACCCTTTTAGTCAGTGTGGAAAATCTCTAGCAGgg
cccgtttaaacccgctgatcagcctcgactgtgccttctagttgccagccatctgttgtttgcccctccc
ccgtgccttccttgaccctggaaggtgccactcccactgtcctttcctaataaaatgaggaaattgcatc
gcattgtctgagtaggtgtcattctattctggggggtggggtggggcaggacagcaagggggaggattgg
gaagacaatagcaggcatgctggggatgcggtgggctctatggcttctgaggcggaaagaaccagctggg
gctctagggggtatccccacgcgccctgtagcggcgcattaagcgcggcgggtgtggtggttacgcgcag
cgtgaccgctacacttgccagcgccctagcgcccgctcctttcgctttcttcccttcctttctcgccacg
ttcgccggctttccccgtcaagctctaaatcgggggctccctttagggttccgatttagtgctttacggc
acctcgaccccaaaaaacttgattagggtgatggttcacgtagtgggccatcgccctgatagacggtttt
tcgccctttgacgttggagtccacgttctttaatagtggactcttgttccaaactggaacaacactcaac
cctatctcggtctattcttttgatttataagggattttgccgatttcggcctattggttaaaaaatgagc
tgatttaacaaaaatttaacgcgaattaattctgtggaatgtgtgtcagttagggtgtggaaagtcccca
ggctccccagcaggcagaagtatgcaaagcatgcatctcaattagtcagcaaccaggtgtggaaagtccc
caggctccccagcaggcagaagtatgcaaagcatgcatctcaattagtcagcaaccatagtcccgcccct
aactccgcccatcccgcccctaactccgcccagttccgcccattctccgccccatggctgactaattttt
tttatttatgcagaggccgaggccgcctctgcctctgagctattccagaagtagtgaggaggcttttttg
gaggcctaggcttttgcaaaaagctcccgggagcttgtatatccattttcggatctgatcagcacgtgtt
gacaattaatcatcggcatagtatatcggcatagtataatacgacaaggtgaggaactaaaccatggcca
agttgaccagtgccgttccggtgctcaccgcgcgcgacgtcgccggagcggtcgagttctggaccgaccg
gctcgggttctcccgggacttcgtggaggacgacttcgccggtgtggtccgggacgacgtgaccctgttc
atcagcgcggtccaggaccaggtggtgccggacaacaccctggcctgggtgtgggtgcgcggcctggacg
agctgtacgccgagtggtcggaggtcgtgtccacgaacttccgggacgcctccgggccggccatgaccga
gatcggcgagcagccgtgggggcgggagttcgccctgcgcgacccggccggcaactgcgtgcacttcgtg
gccgaggagcaggactgacacgtgctacgagatttcgattccaccgccgccttctatgaaaggttgggct
tcggaatcgttttccgggacgccggctggatgatcctccagcgcggggatctcatgctggagttcttcgc
ccaccccaacttgtttattgcagcttataatggttacaaataaagcaatagcatcacaaatttcacaaat
aaagcatttttttcactgcattctagttgtggtttgtccaaactcatcaatgtatcttatcatgtctgta
taccgtcgacctctagctagagcttggcgtaatcatggtcatagctgtttcctgtgtgaaattgttatcc
gctcacaattccacacaacatacgagccggaagcataaagtgtaaagcctggggtgcctaatgagtgagc
taactcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcactga
ctcgctgcgctcggtcgttcggctgcggcgagcggtatcagctcactcaaaggcggtaatacggttatcc
acagaatcaggggataacgcaggaaagaacatgtgagcaaaaggccagcaaaaggccaggaaccgtaaaa
aggccgcgttgctggcgtttttccataggctccgcccccctgacgagcatcacaaaaatcgacgctcaag
tcagaggtggcgaaacccgacaggactataaagataccaggcgtttccccctggaagctccctcgtgcgc
tctcctgttccgaccctgccgcttaccggatacctgtccgcctttctcccttcgggaagcgtggcgcttt
ctcatagctcacgctgtaggtatctcagttcggtgtaggtcgttcgctccaagctgggctgtgtgcacga
accccccgttcagcccgaccgctgcgccttatccggtaactatcgtcttgagtccaacccggtaagacac
gacttatcgccactggcagcagccactggtaacaggattagcagagcgaggtatgtaggcggtgctacag
agttcttgaagtggtggcctaactacggctacactagaagaacagtatttggtatctgcgctctgctgaa
gccagttaccttcggaaaaagagttggtagctcttgatccggcaaacaaaccaccgctggtagcggtggt
ttttttgtttgcaagcagcagattacgcgcagaaaaaaaggatctcaagaagatcctttgatcttttcta
cggggtctgacgctcagtggaacgaaaactcacgttaagggattttggtcatgagattatcaaaaaggat
cttcacctagatccttttaaattaaaaatgaagttttaaatcaatctaaagtatatatgagtaaacttgg
tctgacagttaccaatgcttaatcagtgaggcacctatctcagcgatctgtctatttcgttcatccatag
ttgcctgactccccgtcgtgtagataactacgatacgggagggcttaccatctggccccagtgctgcaat
gataccgcgagacccacgctcaccggctccagatttatcagcaataaaccagccagccggaagggccgag
cgcagaagtggtcctgcaactttatccgcctccatccagtctattaattgttgccgggaagctagagtaa
gtagttcgccagttaatagtttgcgcaacgttgttgccattgctacaggcatcgtggtgtcacgctcgtc
gtttggtatggcttcattcagctccggttcccaacgatcaaggcgagttacatgatcccccatgttgtgc
aaaaaagcggttagctccttcggtcctccgatcgttgtcagaagtaagttggccgcagtgttatcactca
tggttatggcagcactgcataattctcttactgtcatgccatccgtaagatgcttttctgtgactggtga
gtactcaaccaagtcattctgagaatagtgtatgcggcgaccgagttgctcttgcccggcgtcaatacgg
gataataccgcgccacatagcagaactttaaaagtgctcatcattggaaaacgttcttcggggcgaaaac
tctcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaata
agggcgacacggaaatgttgaatactcatactcttcctttttcaatattattgaagcatttatcagggtt
attgtctcatgagcggatacatatttgaatgtatttagaaaaataaacaaataggggttccgcgcacatt
tccccgaaaagtgccacctgac
pBK539 complete sequence, plasmid carried dCas9-DNMT3A fused transgene
linked to GFP selection gene via p2A cleavage signal (nt sequence)
(SEQ ID NO: 40)
gtcgacggatcgggagatctcccgatcccctatggtgcactctcagtacaatctgctctgatgccgcata
gttaagccagtatctgctccctgcttgtgtgttggaggtcgctgagtagtgcgcgagcaaaatttaagct
acaacaaggcaaggcttgaccgacaattgcatgaagaatctgcttagggttaggcgttttgcgctgcttc
gcgatgtacgggccagatatacgcgttgacattgattattgactagttattaatagtaatcaattacggg
gtcattagttcatagcccatatatggagttccgcgttacataacttacggtaaatggcccgcctggctga
ccgcccaacgacccccgcccattgacgtcaataatgacgtatgttcccatagtaacgccaatagggactt
tccattgacgtcaatgggtggagtatttacggtaaactgcccacttggcagtacatcaagtgtatcatat
gccaagtacgccccctattgacgtcaatgacggtaaatggcccgcctggcattatgcccagtacatgacc
ttatgggactttcctacttggcagtacatctacgtattagtcatcgctattaccatggtgatgcggtttt
ggcagtacatcaatgggcgtggatagcggtttgactcacggggatttccaagtctccaccccattgacgt
caatgggagtttgttttggcaccaaaatcaacgggactttccaaaatgtcgtaacaactccgccccattg
acgcaaatgggcggtaggcgtgtacggtgggaggtctatataagcagcgcgttttgcctgtactgggtct
ctctggttagaccagatctgagcctgggagctctctggctaactagggaacccactgcttaagcctcaat
aaagcttgccttgagtgcttcaagtagtgtgtgcccgtctgttgtgtgactctggtaactagagatccct
cagacccttttagtcagtgtggaaaatctctagcagtggcgcccgaacagggacttgaaagcgaaaggga
aaccagaggagctctctcgacgcaggactcggcttgctgaagcgcgcacggcaagaggcgaggggcggcg
actggtgagtacgccaaaaattttgactagcggaggctagaaggagagagatgggtgcgagagcgtcagt
attaagcgggggagaattagatcgcgatgggaaaaaattcggttaaggccagggggaaagaaaaaatata
aattaaaacatatagtatgggcaagcagggagctagaacgattcgcagttaatcctggcctgttagaaac
atcagaaggctgtagacaaatactgggacagctacaaccatcccttcagacaggatcagaagaacttaga
tcattatataatacagtagcaaccctctattgtgtgcatcaaaggatagagataaaagacaccaaggaag
ctttagacaagatagaggaagagcaaaacaaaagtaagaccaccgcacagcaagcggccgctgatcttca
gacctggaggaggagatatgagggacaattggagaagtgaattatataaatataaagtagtaaaaattga
accattaggagtagcacccaccaaggcaaagagaagagtggtgcagagagaaaaaagagcagtgggaata
ggagctttgttccttgggttcttgggagcagcaggaagcactatgggcgcagcgtcaatgacgctgacgg
tacaggccagacaattattgtctggtatagtgcagcagcagaacaatttgctgagggctattgaggcgca
acagcatctgttgcaactcacagtctggggcatcaagcagctccaggcaagaatcctggctgtggaaaga
tacctaaaggatcaacagctcctggggatttggggttgctctggaaaactcatttgcaccactgctgtgc
cttggaatgctagttggagtaataaatctctggaacagatttggaatcacacgacctggatggagtggga
cagagaaattaacaattacacaagcttaatacactccttaattgaagaatcgcaaaaccagcaagaaaag
aatgaacaagaattattggaattagataaatgggcaagtttgtggaattggtttaacataacaaattggc
tgtggtatataaaattattcataatgatagtaggaggcttggtaggtttaagaatagtttttgctgtact
ttctatagtgaatagagttaggcagggatattcaccattatcgtttcagacccacctcccaaccccgagg
ggacccgacaggcccgaaggaatagaagaagaaggtggagagagagacagagacagatccattcgattag
tgaacggatcggcactgcgtgcgccaattctgcagacaaatggcagtattcatccacaattttaaaagaa
aaggggggattggggggtacagtgcaggggaaagaatagtagacataatagcaacagacatacaaactaa
agaattacaaaaacaaattacaaaaattcaaaattttcgggtttattacagggacagcagagatccagtt
tggTTAATTAATGGGCGGGACGTTAACGGGGCGGAACGGTACCgagggcctatttcccatgattccttca
tatttgcatatacgatacaaggctgttagagagataattagaattaatttgactgtaaacacaaagatat
tagtacaaaatacgtgacgtagaaagtaataatttcttgggtagtttgcagttttaaaattatgttttaa
aatggactatcatatgcttaccgtaacttgaaagtatttcgatttcttggctttatatatcttGTGGAAA
GGACGAAAcaccggagacgtgtacacgtctctgTTTtagagctaGAAAtagcaagttaaaataaggctag
tccgttatcaacttgaaaaagtggcaccgagtcggtgcTTTTTTgaattcgctagctaggtcttgaaagg
agtgggaattggctccggtgcccgtcagtgggcagagcgcacatcgcccacagtccccgagaagttgggg
ggaggggtcggcaattgatccggtgcctagagaaggtggcgcggggtaaactgggaaagtgatgtcgtgt
actggctccgcctttttcccgagggtgggggagaaccgtatataagtgcagtagtcgccgtgaacgttct
ttttcgcaacgggtttgccgccagaacacaggaccggtgccaccATGGACTATAAGGACCACGACGGAGA
CTACAAGGATCATGATATTGATTACAAAGACGATGACGATAAGATGGCCCCAAAGAAGAAGCGGAAGGTC
GGTATCCACGGAGTCCCAGCAGCCGACAAGAAGTACAGCATCGGCCTGGCCATCGGCACCAACTCTGTGG
GCTGGGCCGTGATCACCGACGAGTACAAGGTGCCCAGCAAGAAATTCAAGGTGCTGGGCAACACCGACCG
GCACAGCATCAAGAAGAACCTGATCGGAGCCCTGCTGTTCGACAGCGGCGAAACAGCCGAGGCCACCCGG
CTGAAGAGAACCGCCAGAAGAAGATACACCAGACGGAAGAACCGGATCTGCTATCTGCAAGAGATCTTCA
GCAACGAGATGGCCAAGGTGGACGACAGCTTCTTCCACAGACTGGAAGAGTCCTTCCTGGTGGAAGAGGA
TAAGAAGCACGAGCGGCACCCCATCTTCGGCAACATCGTGGACGAGGTGGCCTACCACGAGAAGTACCCC
ACCATCTACCACCTGAGAAAGAAACTGGTGGACAGCACCGACAAGGCCGACCTGCGGCTGATCTATCTGG
CCCTGGCCCACATGATCAAGTTCCGGGGCCACTTCCTGATCGAGGGCGACCTGAACCCCGACAACAGCGA
CGTGGACAAGCTGTTCATCCAGCTGGTGCAGACCTACAACCAGCTGTTCGAGGAAAACCCCATCAACGCC
AGCGGCGTGGACGCCAAGGCCATCCTGTCTGCCAGACTGAGCAAGAGCAGACGGCTGGAAAATCTGATCG
CCCAGCTGCCCGGCGAGAAGAAGAATGGCCTGTTCGGCAACCTGATTGCCCTGAGCCTGGGCCTGACCCC
CAACTTCAAGAGCAACTTCGACCTGGCCGAGGATGCCAAACTGCAGCTGAGCAAGGACACCTACGACGAC
GACCTGGACAACCTGCTGGCCCAGATCGGCGACCAGTACGCCGACCTGTTTCTGGCCGCCAAGAACCTGT
CCGACGCCATCCTGCTGAGCGACATCCTGAGAGTGAACACCGAGATCACCAAGGCCCCCCTGAGCGCCTC
TATGATCAAGAGATACGACGAGCACCACCAGGACCTGACCCTGCTGAAAGCTCTCGTGCGGCAGCAGCTG
CCTGAGAAGTACAAAGAGATTTTCTTCGACCAGAGCAAGAACGGCTACGCCGGCTACATTGACGGCGGAG
CCAGCCAGGAAGAGTTCTACAAGTTCATCAAGCCCATCCTGGAAAAGATGGACGGCACCGAGGAACTGCT
CGTGAAGCTGAACAGAGAGGACCTGCTGCGGAAGCAGCGGACCTTCGACAACGGCAGCATCCCCCACCAG
ATCCACCTGGGAGAGCTGCACGCCATTCTGCGGCGGCAGGAAGATTTTTACCCATTCCTGAAGGACAACC
GGGAAAAGATCGAGAAGATCCTGACCTTCCGCATCCCCTACTACGTGGGCCCTCTGGCCAGGGGAAACAG
CAGATTCGCCTGGATGACCAGAAAGAGCGAGGAAACCATCACCCCCTGGAACTTCGAGGAAGTGGTGGAC
AAGGGCGCTTCCGCCCAGAGCTTCATCGAGCGGATGACCAACTTCGATAAGAACCTGCCCAACGAGAAGG
TGCTGCCCAAGCACAGCCTGCTGTACGAGTACTTCACCGTGTATAACGAGCTGACCAAAGTGAAATACGT
GACCGAGGGAATGAGAAAGCCCGCCTTCCTGAGCGGCGAGCAGAAAAAGGCCATCGTGGACCTGCTGTTC
AAGACCAACCGGAAAGTGACCGTGAAGCAGCTGAAAGAGGACTACTTCAAGAAAATCGAGTGCTTCGACT
CCGTGGAAATCTCCGGCGTGGAAGATCGGTTCAACGCCTCCCTGGGCACATACCACGATCTGCTGAAAAT
TATCAAGGACAAGGACTTCCTGGACAATGAGGAAAACGAGGACATTCTGGAAGATATCGTGCTGACCCTG
ACACTGTTTGAGGACAGAGAGATGATCGAGGAACGGCTGAAAACCTATGCCCACCTGTTCGACGACAAAG
TGATGAAGCAGCTGAAGCGGCGGAGATACACCGGCTGGGGCAGGCTGAGCCGGAAGCTGATCAACGGCAT
CCGGGACAAGCAGTCCGGCAAGACAATCCTGGATTTCCTGAAGTCCGACGGCTTCGCCAACAGAAACTTC
ATGCAGCTGATCCACGACGACAGCCTGACCTTTAAAGAGGACATCCAGAAAGCCCAGGTGTCCGGCCAGG
GCGATAGCCTGCACGAGCACATTGCCAATCTGGCCGGCAGCCCCGCCATTAAGAAGGGCATCCTGCAGAC
AGTGAAGGTGGTGGACGAGCTCGTGAAAGTGATGGGCCGGCACAAGCCCGAGAACATCGTGATCGAAATG
GCCAGAGAGAACCAGACCACCCAGAAGGGACAGAAGAACAGCCGCGAGAGAATGAAGCGGATCGAAGAGG
GCATCAAAGAGCTGGGCAGCCAGATCCTGAAAGAACACCCCGTGGAAAACACCCAGCTGCAGAACGAGAA
GCTGTACCTGTACTACCTGCAGAATGGGCGGGATATGTACGTGGACCAGGAACTGGACATCAACCGGCTG
TCCGACTACGATGTGGACGCTATCGTGCCTCAGAGCTTTCTGAAGGACGACTCCATCGACAACAAGGTGC
TGACCAGAAGCGACAAGAACCGGGGCAAGAGCGACAACGTGCCCTCCGAAGAGGTCGTGAAGAAGATGAA
GAACTACTGGCGGCAGCTGCTGAACGCCAAGCTGATTACCCAGAGAAAGTTCGACAATCTGACCAAGGCC
GAGAGAGGCGGCCTGAGCGAACTGGATAAGGCCGGCTTCATCAAGAGACAGCTGGTGGAAACCCGGCAGA
TCACAAAGCACGTGGCACAGATCCTGGACTCCCGGATGAACACTAAGTACGACGAGAATGACAAGCTGAT
CCGGGAAGTGAAAGTGATCACCCTGAAGTCCAAGCTGGTGTCCGATTTCCGGAAGGATTTCCAGTTTTAC
AAAGTGCGCGAGATCAACAACTACCACCACGCCCACGACGCCTACCTGAACGCCGTCGTGGGAACCGCCC
TGATCAAAAAGTACCCTAAGCTGGAAAGCGAGTTCGTGTACGGCGACTACAAGGTGTACGACGTGCGGAA
GATGATCGCCAAGAGCGAGCAGGAAATCGGCAAGGCTACCGCCAAGTACTTCTTCTACAGCAACATCATG
AACTTTTTCAAGACCGAGATTACCCTGGCCAACGGCGAGATCCGGAAGCGGCCTCTGATCGAGACAAACG
GCGAAACCGGGGAGATCGTGTGGGATAAGGGCCGGGATTTTGCCACCGTGCGGAAAGTGCTGAGCATGCC
CCAAGTGAATATCGTGAAAAAGACCGAGGTGCAGACAGGCGGCTTCAGCAAAGAGTCTATCCTGCCCAAG
AGGAACAGCGATAAGCTGATCGCCAGAAAGAAGGACTGGGACCCTAAGAAGTACGGCGGCTTCGACAGCC
CCACCGTGGCCTATTCTGTGCTGGTGGTGGCCAAAGTGGAAAAGGGCAAGTCCAAGAAACTGAAGAGTGT
GAAAGAGCTGCTGGGGATCACCATCATGGAAAGAAGCAGCTTCGAGAAGAATCCCATCGACTTTCTGGAA
GCCAAGGGCTACAAAGAAGTGAAAAAGGACCTGATCATCAAGCTGCCTAAGTACTCCCTGTTCGAGCTGG
AAAACGGCCGGAAGAGAATGCTGGCCTCTGCCGGCGAACTGCAGAAGGGAAACGAACTGGCCCTGCCCTC
CAAATATGTGAACTTCCTGTACCTGGCCAGCCACTATGAGAAGCTGAAGGGCTCCCCCGAGGATAATGAG
CAGAAACAGCTGTTTGTGGAACAGCACAAGCACTACCTGGACGAGATCATCGAGCAGATCAGCGAGTTCT
CCAAGAGAGTGATCCTGGCCGACGCTAATCTGGACAAAGTGCTGTCCGCCTACAACAAGCACCGGGATAA
GCCCATCAGAGAGCAGGCCGAGAATATCATCCACCTGTTTACCCTGACCAATCTGGGAGCCCCTGCCGCC
TTCAAGTACTTTGACACCACCATCGACCGGAAGAGGTACACCAGCACCAAAGAGGTGCTGGACGCCACCC
TGATCCACCAGAGCATCACCGGCCTGTACGAGACACGGATCGACCTGTCTCAGCTGGGAGGCGACAAAAG
GCCGGCGGCCACGAAAAAGGCCGGACAGGCCAAAAAGAAAAAGCTCGAGGGCGGAGGCGGGAGCGGATCC
CCCTCCCGGCTCCAGATGttcttcgctaataaccacgaccaggaatttgaccctccaaaggtttacccac
ctgtcccagctgagaagaggaagcccatccgggtgctgtctctctttgatggaatcgctacagggctcct
ggtgctgaaggacttgggcattcaggtggaccgctacattgcctcggaggtgtgtgaggactccatcacg
gtgggcatggtgcggcaccaggggaagatcatgtacgtcggggacgtccgcagcgtcacacagaagcata
tccaggagtggggcccattcgatctggtgattgggggcagtccctgcaatgacctctccatcgtcaaccc
tgctcgcaagggcctctacgagggcactggccggctcttctttgagttctaccgcctcctgcatgatgcg
cggcccaaggagggagatgatcgccccttcttctggctctttgagaatgtggtggccatgggcgttagtg
acaagagggacatctcgcgatttctcgagtccaaccctgtgatgattgatgccaaagaagtgtcagctgc
acacagggcccgctacttctggggtaaccttcccggtatgaacaggccgttggcatccactgtgaatgat
aagctggagctgcaggagtgtctggagcatggcaggatagccaagttcagcaaagtgaggaccattacta
cgaggtcaaactccataaagcagggcaaaGACCAGCATTTTCCTGTGITCATGAATGAGAAAGAGgacat
cttatggtgcactgaaatggaaagggtatttggtttcccagtccactatactgacgtgtccaacatgagc
cgcttggcgaggcagagactgctgggccggtcatggagcgtgccagtcatccgccacctcttcgctcCGC
TGAAGGAGTATTTTGCGTGTGTGtccggccggggccggcccggatccggcgcaacaaacttctctctgct
gaaacaagccggagatgtcgaagagaatcctggaccgATGGTGAGCAAGGGCGAGgagctgttcaccggg
gtggtgcccatcctggtcgagctggacggcgacgtaaacggccacaagttcagcgtgtccggcgagggcg
agggcgatgccacctacggcaagctgaccctgaagttcatctgcaccaccggcaagctgcccgtgccctg
gcccaccctcgtgaccaccctgacctacggcgtgcagtgcttcagccgctaccccgaccacatgaagcag
cacgacttcttcaagtccgccatgcccgaaggctacgtccaggagcgcaccatcttcttcaaggacgacg
gcaactacaagacccgcgccgaggtgaagttcgagggcgacaccctggtgaaccgcatcgagctgaaggg
catcgacttcaaggaggacggcaacatcctggggcacaagctggagtacaactacaacagccacaacgtc
tatatcatggccgacaagcagaagaacggcatcaaggtgaacttcaagatccgccacaacatcgaggacg
gcagcgtgcagctcgccgaccactaccagcagaacacccccatcggcgacggccccgtgctgctgcccga
caaccactacctgagcacccagtccgccctgagcaaagaccccaacgagaagcgcgatcacatggtcctg
ctggagttcgtgaccgccgccgggatcactctcggcatggacgagctgtacaagtaaagcggccgcgtcg
acaatcaacctctggattacaaaatttgtgaaagattgactggtattcttaactatgttgctccttttac
gctatgtggatacgctgctttaatgcctttgtatcatgctattgcttcccgtatggctttcattttctcc
tccttgtataaatcctggttgctgtctctttatgaggagttgtggcccgttgtcaggcaacgtggcgtgg
tgtgcactgtgtttgctgacgcaacccccactggttggggcattgccaccacctgtcagctcctttccgg
gactttcgctttcccactccctattgccacggcggaactcatcgccgcctgccttgcccgctgctggaca
ggggctcggctgttgggcactgacaattccgtggtgttgtcggggaagctgacgtcctttccatggctgc
tcgcctgtgttgccacctggattctgcgcgggacgtccttctgctacgtcccttcggccctcaatccagc
ggaccttccttcccgcggcctgctgccggctctgcggcctcttccgcgtcttcgccttcgccctcagacg
agtcggatctccctttgggccgcctccccgcctggaattcgagctcggtacctttaagaccaatgactta
caaggcagctgtagatcttagccactttttaaaagaaaaggggggactggaagggctaattcactcccaa
cgaagacaagatctgctttttgcttgtactgggtctctctggttagaccagatctgagcctgggagctct
ctggctaactagggaacccactgcttaagcctcaataaagcttgccttgagtgcttcaagtagtgtgtgc
ccgtctgttgtgtgactctggtaactagagatccctcagacccttttagtcagtgtggaaaatctctagc
agtagtagttcatgtcatcttattattcagtatttataacttgcaaagaaatgaatatcagagagtgaga
ggaacttgtttattgcagcttataatggttacaaataaagcaatagcatcacaaatttcacaaataaagc
atttttttcactgcattctagttgtggtttgtccaaactcatcaatgtatcttatcatgtctggctctag
ctatcccgcccctaactccgcccatcccgcccctaactccgcccagttccgcccattctccgccccatgg
ctgactaattttttttatttatgcagaggccgaggccgcctcggcctctgagctattccagaagtagtga
ggaggcttttttggaggcctagggacgtacccaattcgccctatagtgagtcgtattacgcgcgctcact
ggccgtcgttttacaacgtcgtgactgggaaaaccctggcgttacccaacttaatcgccttgcagcacat
ccccctttcgccagctggcgtaatagcgaagaggcccgcaccgatcgcccttcccaacagttgcgcagcc
tgaatggcgaatgggacgcgccctgtagcggcgcattaagcgcggcgggtgtggtggttacgcgcagcgt
gaccgctacacttgccagcgccctagcgcccgctcctttcgctttcttcccttcctttctcgccacgttc
gccggctttccccgtcaagctctaaatcgggggctccctttagggttccgatttagtgctttacggcacc
togaccccaaaaaacttgattagggtgatggttcacgtagtgggccatcgccctgatagacggtttttcg
ccatttgacgttggagtccacgttctttaatagtggactcttgttccaaactggaacaacactcaaccct
atctcggtctattcttttgatttataagggattttgccgatttcggcctattggttaaaaaatgagctga
tttaacaaaaatttaacgCGAATTTTAACAAAATATTAACGCTTACAATTTAGGTGccggccatgaccga
gatcggcgagcagccgtgggggcgggagttcgccctgcgcgacccggccggcaactgcgtgcacttcgtg
gccgaggagcaggactgacacgtgctacgagatttcgattccaccgccgccttctatgaaaggttgggct
tcggaatcgttttccgggacgccggctggatgatcctccagcgcggggatctcatgctggagttcttcgc
ccaccccaacttgtttattgcagcttataatggttacaaataaagcaatagcatcacaaatttcacaaat
aaagcatttttttcactgcattctagttgtggtttgtccaaactcatcaatgtatcttatcatgtctgta
taccgtcgacctctagctagagcttggcgtaatcatggtcatagctgtttcctgtgtgaaattgttatcc
gctcacaattccacacaacatacgagccggaagcataaagtgtaaagcctggggtgcctaatgagtgagc
taactcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcactga
ctcgctgcgctcggtcgttcggctgcggcgagcggtatcagctcactcaaaggcggtaatacggttatcc
acagaatcaggggataacgcaggaaagaacatgtgagcaaaaggccagcaaaaggccaggaaccgtaaaa
aggccgcgttgctggcgtttttccataggctccgcccccctgacgagcatcacaaaaatcgacgctcaag
tcagaggtggcgaaacccgacaggactataaagataccaggcgtttccccctggaagctccctcgtgcgc
tctcctgttccgaccctgccgcttaccggatacctgtccgcctttctcccttcgggaagcgtggcgcttt
ctcatagctcacgctgtaggtatctcagttcggtgtaggtcgttcgctccaagctgggctgtgtgcacga
accccccgttcagcccgaccgctgcgccttatccggtaactatcgtcttgagtccaacccggtaagacac
gacttatcgccactggcagcagccactggtaacaggattagcagagcgaggtatgtaggcggtgctacag
agttcttgaagtggtggcctaactacggctacactagaagaacagtatttggtatctgcgctctgctgaa
gccagttaccttcggaaaaagagttggtagctcttgatccggcaaacaaaccaccgctggtagcggtggt
ttttttgtttgcaagcagcagattacgcgcagaaaaaaaggatctcaagaagatcctttgatcttttcta
cggggtctgacgctcagtggaacgaaaactcacgttaagggattttggtcatgagattatcaaaaaggat
cttcacctagatccttttaaattaaaaatgaagttttaaatcaatctaaagtatatatgagtaaacttgg
tctgacagttaccaatgcttaatcagtgaggcacctatctcagcgatctgtctatttcgttcatccatag
ttgcctgactccccgtcgtgtagataactacgatacgggagggcttaccatctggccccagtgctgcaat
gataccgcgagacccacgctcaccggctccagatttatcagcaataaaccagccagccggaagggccgag
cgcagaagtggtcctgcaactttatccgcctccatccagtctattaattgttgccgggaagctagagtaa
gtagttcgccagttaatagtttgcgcaacgttgttgccattgctacaggcatcgtggtgtcacgctcgtc
gtttggtatggcttcattcagctccggttcccaacgatcaaggcgagttacatgatcccccatgttgtgc
aaaaaagcggttagctccttcggtcctccgatcgttgtcagaagtaagttggccgcagtgttatcactca
tggttatggcagcactgcataattctcttactgtcatgccatccgtaagatgcttttctgtgactggtga
gtactcaaccaagtcattctgagaatagtgtatgcggcgaccgagttgctcttgcccggcgtcaatacgg
gataataccgcgccacatagcagaactttaaaagtgctcatcattggaaaacgttcttcggggcgaaaac
tctcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaata
agggcgacacggaaatgttgaatactcatactcttcctttttcaatattattgaagcatttatcagggtt
attgtctcatgagcggatacatatttgaatgtatttagaaaaataaacaaataggggttccgcgcacatt
tccccgaaaagtgccacctgac
pBK744 complete sequence, plasmid carried dCas9-DNMT3A fused transgene
linked to GFP selection gene via p2A cleavage signal. The plasmid
carried gRNA3 (see FIG. 8) targeting rat/mouse intron Snca-intron 1
sequences (nt sequence)
(SEQ ID NO: 41)
gtcgacggatcgggagatctcccgatcccctatggtgcactctcagtacaatctgctctgatgccgcata
gttaagccagtatctgctccctgcttgtgtgttggaggtcgctgagtagtgcgcgagcaaaatttaagct
acaacaaggcaaggcttgaccgacaattgcatgaagaatctgcttagggttaggcgttttgcgctgcttc
gcgatgtacgggccagatatacgcgttgacattgattattgactagttattaatagtaatcaattacggg
gtcattagttcatagcccatatatggagttccgcgttacataacttacggtaaatggcccgcctggctga
ccgcccaacgacccccgcccattgacgtcaataatgacgtatgttcccatagtaacgccaatagggactt
tccattgacgtcaatgggtggagtatttacggtaaactgcccacttggcagtacatcaagtgtatcatat
gccaagtacgccccctattgacgtcaatgacggtaaatggcccgcctggcattatgcccagtacatgacc
ttatgggactttcctacttggcagtacatctacgtattagtcatcgctattaccatggtgatgcggtttt
ggcagtacatcaatgggcgtggatagcggtttgactcacggggatttccaagtctccaccccattgacgt
caatgggagtttgttttggcaccaaaatcaacgggactttccaaaatgtcgtaacaactccgccccattg
acgcaaatgggcggtaggcgtgtacggtgggaggtctatataagcagcgcgttttgcctgtactgggtct
ctctggttagaccagatctgagcctgggagctctctggctaactagggaacccactgcttaagcctcaat
aaagcttgccttgagtgcttcaagtagtgtgtgcccgtctgttgtgtgactctggtaactagagatccct
cagacccttttagtcagtgtggaaaatctctagcagtggcgcccgaacagggacttgaaagcgaaaggga
aaccagaggagctctctcgacgcaggactcggcttgctgaagcgcgcacggcaagaggcgaggggcggcg
actggtgagtacgccaaaaattttgactagcggaggctagaaggagagagatgggtgcgagagcgtcagt
attaagcgggggagaattagatcgcgatgggaaaaaattcggttaaggccagggggaaagaaaaaatata
aattaaaacatatagtatgggcaagcagggagctagaacgattcgcagttaatcctggcctgttagaaac
atcagaaggctgtagacaaatactgggacagctacaaccatcccttcagacaggatcagaagaacttaga
tcattatataatacagtagcaaccctctattgtgtgcatcaaaggatagagataaaagacaccaaggaag
ctttagacaagatagaggaagagcaaaacaaaagtaagaccaccgcacagcaagcggccgctgatcttca
gacctggaggaggagatatgagggacaattggagaagtgaattatataaatataaagtagtaaaaattga
accattaggagtagcacccaccaaggcaaagagaagagtggtgcagagagaaaaaagagcagtgggaata
ggagctttgttccttgggttcttgggagcagcaggaagcactatgggcgcagcgtcaatgacgctgacgg
tacaggccagacaattattgtctggtatagtgcagcagcagaacaatttgctgagggctattgaggcgca
acagcatctgttgcaactcacagtctggggcatcaagcagctccaggcaagaatcctggctgtggaaaga
tacctaaaggatcaacagctcctggggatttggggttgctctggaaaactcatttgcaccactgctgtgc
cttggaatgctagttggagtaataaatctctggaacagatttggaatcacacgacctggatggagtggga
cagagaaattaacaattacacaagcttaatacactccttaattgaagaatcgcaaaaccagcaagaaaag
aatgaacaagaattattggaattagataaatgggcaagtttgtggaattggtttaacataacaaattggc
tgtggtatataaaattattcataatgatagtaggaggcttggtaggtttaagaatagtttttgctgtact
ttctatagtgaatagagttaggcagggatattcaccattatcgtttcagacccacctcccaaccccgagg
ggacccgacaggcccgaaggaatagaagaagaaggtggagagagagacagagacagatccattcgattag
tgaacggatcggcactgcgtgcgccaattctgcagacaaatggcagtattcatccacaattttaaaagaa
aaggggggattggggggtacagtgcaggggaaagaatagtagacataatagcaacagacatacaaactaa
agaattacaaaaacaaattacaaaaattcaaaattttcgggtttattacagggacagcagagatccagtt
tggTTAATTAATGGGCGGGACGTTAACGGGGCGGAACGGTACCgagggcctatttcccatgattccttca
tatttgcatatacgatacaaggctgttagagagataattagaattaatttgactgtaaacacaaagatat
tagtacaaaatacgtgacgtagaaagtaataatttcttgggtagtttgcagttttaaaattatgttttaa
aatggactatcatatgcttaccgtaacttgaaagtatttcgatttcttggctttatatatcttGTGGAAA
GGACGAAAcaccgTTTTTCAAGCGGAAACGCTAgTTTtagagctaGAAAtagcaagttaaaataaggcta
gtccgttatcaacttgaaaaagtggcaccgagtcggtgcTTTTTTgaattcgctagctaggtcttgaaag
gagtgggaattggctccggtgcccgtcagtgggcagagcgcacatcgcccacagtccccgagaagttggg
gggaggggtcggcaattgatccggtgcctagagaaggtggcgcggggtaaactgggaaagtgatgtcgtg
tactggctccgcctttttcccgagggtgggggagaaccgtatataagtgcagtagtcgccgtgaacgttc
tttttcgcaacgggtttgccgccagaacacaggaccggtgccaccATGGACTATAAGGACCACGACGGAG
ACTACAAGGATCATGATATTGATTACAAAGACGATGACGATAAGATGGCCCCAAAGAAGAAGCGGAAGGT
CGGTATCCACGGAGTCCCAGCAGCCGACAAGAAGTACAGCATCGGCCTGGCCATCGGCACCAACTCTGTG
GGCTGGGCCGTGATCACCGACGAGTACAAGGTGCCCAGCAAGAAATTCAAGGTGCTGGGCAACACCGACC
GGCACAGCATCAAGAAGAACCTGATCGGAGCCCTGCTGTTCGACAGCGGCGAAACAGCCGAGGCCACCCG
GCTGAAGAGAACCGCCAGAAGAAGATACACCAGACGGAAGAACCGGATCTGCTATCTGCAAGAGATCTTC
AGCAACGAGATGGCCAAGGTGGACGACAGCTTCTTCCACAGACTGGAAGAGTCCTTCCTGGTGGAAGAGG
ATAAGAAGCACGAGCGGCACCCCATCTTCGGCAACATCGTGGACGAGGTGGCCTACCACGAGAAGTACCC
CACCATCTACCACCTGAGAAAGAAACTGGTGGACAGCACCGACAAGGCCGACCTGCGGCTGATCTATCTG
GCCCTGGCCCACATGATCAAGTTCCGGGGCCACTTCCTGATCGAGGGCGACCTGAACCCCGACAACAGCG
ACGTGGACAAGCTGTTCATCCAGCTGGTGCAGACCTACAACCAGCTGTTCGAGGAAAACCCCATCAACGC
CAGCGGCGTGGACGCCAAGGCCATCCTGTCTGCCAGACTGAGCAAGAGCAGACGGCTGGAAAATCTGATC
GCCCAGCTGCCCGGCGAGAAGAAGAATGGCCTGTTCGGCAACCTGATTGCCCTGAGCCTGGGCCTGACCC
CCAACTTCAAGAGCAACTTCGACCTGGCCGAGGATGCCAAACTGCAGCTGAGCAAGGACACCTACGACGA
CGACCTGGACAACCTGCTGGCCCAGATCGGCGACCAGTACGCCGACCTGTTTCTGGCCGCCAAGAACCTG
TCCGACGCCATCCTGCTGAGCGACATCCTGAGAGTGAACACCGAGATCACCAAGGCCCCCCTGAGCGCCT
CTATGATCAAGAGATACGACGAGCACCACCAGGACCTGACCCTGCTGAAAGCTCTCGTGCGGCAGCAGCT
GCCTGAGAAGTACAAAGAGATTTTCTTCGACCAGAGCAAGAACGGCTACGCCGGCTACATTGACGGCGGA
GCCAGCCAGGAAGAGTTCTACAAGTTCATCAAGCCCATCCTGGAAAAGATGGACGGCACCGAGGAACTGC
TCGTGAAGCTGAACAGAGAGGACCTGCTGCGGAAGCAGCGGACCTTCGACAACGGCAGCATCCCCCACCA
GATCCACCTGGGAGAGCTGCACGCCATTCTGCGGCGGCAGGAAGATTTTTACCCATTCCTGAAGGACAAC
CGGGAAAAGATCGAGAAGATCCTGACCTTCCGCATCCCCTACTACGTGGGCCCTCTGGCCAGGGGAAACA
GCAGATTCGCCTGGATGACCAGAAAGAGCGAGGAAACCATCACCCCCTGGAACTTCGAGGAAGTGGTGGA
CAAGGGCGCTTCCGCCCAGAGCTTCATCGAGCGGATGACCAACTTCGATAAGAACCTGCCCAACGAGAAG
GTGCTGCCCAAGCACAGCCTGCTGTACGAGTACTTCACCGTGTATAACGAGCTGACCAAAGTGAAATACG
TGACCGAGGGAATGAGAAAGCCCGCCTTCCTGAGCGGCGAGCAGAAAAAGGCCATCGTGGACCTGCTGTT
CAAGACCAACCGGAAAGTGACCGTGAAGCAGCTGAAAGAGGACTACTTCAAGAAAATCGAGTGCTTCGAC
TCCGTGGAAATCTCCGGCGTGGAAGATCGGTTCAACGCCTCCCTGGGCACATACCACGATCTGCTGAAAA
TTATCAAGGACAAGGACTTCCTGGACAATGAGGAAAACGAGGACATTCTGGAAGATATCGTGCTGACCCT
GACACTGTTTGAGGACAGAGAGATGATCGAGGAACGGCTGAAAACCTATGCCCACCTGTTCGACGACAAA
GTGATGAAGCAGCTGAAGCGGCGGAGATACACCGGCTGGGGCAGGCTGAGCCGGAAGCTGATCAACGGCA
TCCGGGACAAGCAGTCCGGCAAGACAATCCTGGATTTCCTGAAGTCCGACGGCTTCGCCAACAGAAACTT
CATGCAGCTGATCCACGACGACAGCCTGACCTTTAAAGAGGACATCCAGAAAGCCCAGGTGTCCGGCCAG
GGCGATAGCCTGCACGAGCACATTGCCAATCTGGCCGGCAGCCCCGCCATTAAGAAGGGCATCCTGCAGA
CAGTGAAGGTGGTGGACGAGCTCGTGAAAGTGATGGGCCGGCACAAGCCCGAGAACATCGTGATCGAAAT
GGCCAGAGAGAACCAGACCACCCAGAAGGGACAGAAGAACAGCCGCGAGAGAATGAAGCGGATCGAAGAG
GGCATCAAAGAGCTGGGCAGCCAGATCCTGAAAGAACACCCCGTGGAAAACACCCAGCTGCAGAACGAGA
AGCTGTACCTGTACTACCTGCAGAATGGGCGGGATATGTACGTGGACCAGGAACTGGACATCAACCGGCT
GTCCGACTACGATGTGGACGCTATCGTGCCTCAGAGCTTTCTGAAGGACGACTCCATCGACAACAAGGTG
CTGACCAGAAGCGACAAGAACCGGGGCAAGAGCGACAACGTGCCCTCCGAAGAGGTCGTGAAGAAGATGA
AGAACTACTGGCGGCAGCTGCTGAACGCCAAGCTGATTACCCAGAGAAAGTTCGACAATCTGACCAAGGC
CGAGAGAGGCGGCCTGAGCGAACTGGATAAGGCCGGCTTCATCAAGAGACAGCTGGTGGAAACCCGGCAG
ATCACAAAGCACGTGGCACAGATCCTGGACTCCCGGATGAACACTAAGTACGACGAGAATGACAAGCTGA
TCCGGGAAGTGAAAGTGATCACCCTGAAGTCCAAGCTGGTGTCCGATTTCCGGAAGGATTTCCAGTTTTA
CAAAGTGCGCGAGATCAACAACTACCACCACGCCCACGACGCCTACCTGAACGCCGTCGTGGGAACCGCC
CTGATCAAAAAGTACCCTAAGCTGGAAAGCGAGTTCGTGTACGGCGACTACAAGGTGTACGACGTGCGGA
AGATGATCGCCAAGAGCGAGCAGGAAATCGGCAAGGCTACCGCCAAGTACTTCTTCTACAGCAACATCAT
GAACTTTTTCAAGACCGAGATTACCCTGGCCAACGGCGAGATCCGGAAGCGGCCTCTGATCGAGACAAAC
GGCGAAACCGGGGAGATCGTGTGGGATAAGGGCCGGGATTTTGCCACCGTGCGGAAAGTGCTGAGCATGC
CCCAAGTGAATATCGTGAAAAAGACCGAGGTGCAGACAGGCGGCTTCAGCAAAGAGTCTATCCTGCCCAA
GAGGAACAGCGATAAGCTGATCGCCAGAAAGAAGGACTGGGACCCTAAGAAGTACGGCGGCTTCGACAGC
CCCACCGTGGCCTATTCTGTGCTGGTGGTGGCCAAAGTGGAAAAGGGCAAGTCCAAGAAACTGAAGAGTG
TGAAAGAGCTGCTGGGGATCACCATCATGGAAAGAAGCAGCTTCGAGAAGAATCCCATCGACTTTCTGGA
AGCCAAGGGCTACAAAGAAGTGAAAAAGGACCTGATCATCAAGCTGCCTAAGTACTCCCTGTTCGAGCTG
GAAAACGGCCGGAAGAGAATGCTGGCCTCTGCCGGCGAACTGCAGAAGGGAAACGAACTGGCCCTGCCCT
CCAAATATGTGAACTTCCTGTACCTGGCCAGCCACTATGAGAAGCTGAAGGGCTCCCCCGAGGATAATGA
GCAGAAACAGCTGTTTGTGGAACAGCACAAGCACTACCTGGACGAGATCATCGAGCAGATCAGCGAGTTC
TCCAAGAGAGTGATCCTGGCCGACGCTAATCTGGACAAAGTGCTGTCCGCCTACAACAAGCACCGGGATA
AGCCCATCAGAGAGCAGGCCGAGAATATCATCCACCTGTTTACCCTGACCAATCTGGGAGCCCCTGCCGC
CTTCAAGTACTTTGACACCACCATCGACCGGAAGAGGTACACCAGCACCAAAGAGGTGCTGGACGCCACC
CTGATCCACCAGAGCATCACCGGCCTGTACGAGACACGGATCGACCTGTCTCAGCTGGGAGGCGACAAAA
GGCCGGCGGCCACGAAAAAGGCCGGACAGGCCAAAAAGAAAAAGCTCGAGGGCGGAGGCGGGAGCGGATC
CCCCTCCCGGCTCCAGATGttcttcgctaataaccacgaccaggaatttgaccctccaaaggtttaccca
cctgtcccagctgagaagaggaagcccatccgggtgctgtctctctttgatggaatcgctacagggctcc
tggtgctgaaggacttgggcattcaggtggaccgctacattgcatcggaggtgtgtgaggactccatcac
ggtgggcatggtgcggcaccaggggaagatcatgtacgtcggggacgtccgcagcgtcacacagaagcat
atccaggagtggggcccattcgatctggtgattgggggcagtccctgcaatgacctctccatcgtcaacc
ctgctcgcaagggcctctacgagggcactggccggctcttctttgagttctaccgcctcctgcatgatgc
gcggcccaaggagggagatgatcgccccttcttctggctctttgagaatgtggtggccatgggcgttagt
gacaagagggacatctcgcgatttctcgagtccaaccctgtgatgattgatgccaaagaagtgtcagctg
cacacagggcccgctacttctggggtaaccttcccggtatgaacaggccgttggcatccactgtgaatga
taagctggagctgcaggagtgtctggagcatggcaggatagccaagttcagcaaagtgaggaccattact
acgaggtcaaactccataaagcagggcaaaGACCAGCATTTTCCIGTGTTCATGAATGAGAAAGAGgaca
tcttatggtgcactgaaatggaaagggtatttggtttcccagtccactatactgacgtgtccaacatgag
ccgcttggcgaggcagagactgctgggccggtcatggagcgtgccagtcatccgccacctcttcgctcCG
CTGAAGGAGTATTTTGCGTGTGTGtccggccggggccggcccggatccggcgcaacaaacttctctctgc
tgaaacaagccggagatgtcgaagagaatcctggaccgATGGTGAGCAAGGGCGAGgagctgttcaccgg
ggtggtgcccatcctggtcgagctggacggcgacgtaaacggccacaagttcagcgtgtccggcgagggc
gagggcgatgccacctacggcaagctgaccctgaagttcatctgcaccaccggcaagctgcccgtgccct
ggcccaccctcgtgaccaccctgacctacggcgtgcagtgcttcagccgctaccccgaccacatgaagca
gcacgacttcttcaagtccgccatgcccgaaggctacgtccaggagcgcaccatcttcttcaaggacgac
ggcaactacaagacccgcgccgaggtgaagttcgagggcgacaccctggtgaaccgcatcgagctgaagg
gcatcgacttcaaggaggacggcaacatcctggggcacaagctggagtacaactacaacagccacaacgt
ctatatcatggccgacaagcagaagaacggcatcaaggtgaacttcaagatccgccacaacatcgaggac
ggcagcgtgcagctcgccgaccactaccagcagaacacccccatcggcgacggccccgtgctgctgcccg
acaaccactacctgagcacccagtccgccctgagcaaagaccccaacgagaagcgcgatcacatggtcct
gctggagttcgtgaccgccgccgggatcactctcggcatggacgagctgtacaagtaaagcggccgcgtc
gacaatcaacctctggattacaaaatttgtgaaagattgactggtattcttaactatgttgctcctttta
cgctatgtggatacgctgctttaatgcctttgtatcatgctattgcttcccgtatggctttcattttctc
ctccttgtataaatcctggttgctgtctctttatgaggagttgtggcccgttgtcaggcaacgtggcgtg
gtgtgcactgtgtttgctgacgcaacccccactggttggggcattgccaccacctgtcagctcctttccg
ggactttcgctttccccctccctattgccacggcggaactcatcgccgcctgccttgcccgctgctggac
aggggctcggctgttgggcactgacaattccgtggtgttgtcggggaagctgacgtcctttccatggctg
ctcgcctgtgttgccacctggattctgcgcgggacgtccttctgctacgtcccttcggccctcaatccag
cggaccttccttcccgcggcctgctgccggctctgcggcctcttccgcgtcttcgccttcgccctcagac
gagtcggatctccctttgggccgcctccccgcctggaattcgagctcggtacctttaagaccaatgactt
acaaggcagctgtagatcttagccactttttaaaagaaaaggggggactggaagggctaattcactocca
acgaagacaagatctgctttttgcttgtactgggtctctctggttagaccagatctgagcctgggagctc
tctggctaactagggaacccactgcttaagcctcaataaagcttgccttgagtgcttcaagtagtgtgtg
cccgtctgttgtgtgactctggtaactagagatccctcagacccttttagtcagtgtggaaaatctctag
cagtagtagttcatgtcatcttattattcagtatttataacttgcaaagaaatgaatatcagagagtgag
aggaacttgtttattgcagcttataatggttacaaataaagcaatagcatcacaaatttcacaaataaag
catttttttcactgcattctagttgtggtttgtccaaactcatcaatgtatcttatcatgtctggctcta
gctatcccgcccctaactccgcccatcccgcccctaactccgcccagttccgcccattctccgccccatg
gctgactaattttttttatttatgcagaggccgaggccgcctcggcctctgagctattccagaagtagtg
aggaggcttttttggaggcctagggacgtacccaattcgccctatagtgagtcgtattacgcgcgctcac
tggccgtcgttttacaacgtcgtgactgggaaaaccctggcgttacccaacttaatcgccttgcagcaca
tccccctttcgccagctggcgtaatagcgaagaggcccgcaccgatcgcccttcccaacagttgcgcagc
ctgaatggcgaatgggacgcgccctgtagcggcgcattaagcgcggcgggtgtggtggttacgcgcagcg
tgaccgctacacttgccagcgccctagcgcccgctcctttcgctttcttcccttcctttctcgccacgtt
cgccggctttccccgtcaagctctaaatcgggggctccctttagggttccgatttagtgctttacggcac
ctcgaccccaaaaaacttgattagggtgatggttcacgtagtgggccatcgccctgatagacggtttttc
gccctttgacgttggagtccacgttctttaatagtggactattgttccaaactggaacaacactcaaccc
tatctcggtctattcttttgatttataagggattttgccgatttcggcctattggttaaaaaatgagctg
atttaacaaaaatttaacgCGAATTTTAACAAAATATTAACGCTTACAATTTAGGTGccggccatgaccg
agatcggcgagcagccgtgggggcgggagttcgccctgcgcgacccggccggcaactgcgtgcacttcgt
ggccgaggagcaggactgacacgtgctacgagatttcgattccaccgccgccttctatgaaaggttgggc
ttcggaatcgttttccgggacgccggctggatgatcctccagcgcggggatctcatgctggagttcttcg
cccaccccaacttgtttattgcagcttataatggttacaaataaagcaatagcatcacaaatttcacaaa
taaagcatttttttcactgcattctagttgtggtttgtccaaactcatcaatgtatcttatcatgtctgt
ataccgtcgacctctagctagagcttggcgtaatcatggtcatagctgtttcctgtgtgaaattgttatc
cgctcacaattccacacaacatacgagccggaagcataaagtgtaaagcctggggtgcctaatgagtgag
ctaactcacattaattgcgttgcgctcactgcccgctttccagtcgggaaacctgtcgtgccagctgcat
taatgaatcggccaacgcgcggggagaggcggtttgcgtattgggcgctcttccgcttcctcgctcactg
actcgctgcgctcggtcgttcggctgcggcgagcggtatcagctcactcaaaggcggtaatacggttatc
cacagaatcaggggataacgcaggaaagaacatgtgagcaaaaggccagcaaaaggccaggaaccgtaaa
aaggccgcgttgctggcgtttttccataggctccgcccccctgacgagcatcacaaaaatcgacgctcaa
gtcagaggtggcgaaacccgacaggactataaagataccaggcgtttccccctggaagctccctcgtgcg
ctctcctgttccgaccctgccgcttaccggatacctgtccgcctttctcccttcgggaagcgtggcgctt
tctcatagctcacgctgtaggtatctcagttcggtgtaggtcgttcgctccaagctgggctgtgtgcacg
aaccccccgttcagcccgaccgctgcgccttatccggtaactatcgtcttgagtccaacccggtaagaca
cgacttatcgccactggcagcagccactggtaacaggattagcagagcgaggtatgtaggcggtgctaca
gagttcttgaagtggtggcctaactacggctacactagaagaacagtatttggtatctgcgctctgctga
agccagttaccttcggaaaaagagttggtagctcttgatccggcaaacaaaccaccgctggtagcggtgg
tttttttgtttgcaagcagcagattacgcgcagaaaaaaaggatctcaagaagatcctttgatcttttct
acggggtctgacgctcagtggaacgaaaactcacgttaagggattttggtcatgagattatcaaaaagga
tcttcacctagatccttttaaattaaaaatgaagttttaaatcaatctaaagtatatatgagtaaacttg
gtctgacagttaccaatgcttaatcagtgaggcacctatctcagcgatctgtctatttcgttcatccata
gttgcctgactccccgtcgtgtagataactacgatacgggagggcttaccatctggccccagtgctgcaa
tgataccgcgagacccacgctcaccggctccagatttatcagcaataaaccagccagccggaagggccga
gcgcagaagtggtcctgcaactttatccgcctccatccagtctattaattgttgccgggaagctagagta
agtagttcgccagttaatagtttgcgcaacgttgttgccattgctacaggcatcgtggtgtcacgctcgt
cgtttggtatggcttcattcagctccggttcccaacgatcaaggcgagttacatgatccaccatgttgtg
caaaaaagcggttagctccttcggtcctccgatcgttgtcagaagtaagttggccgcagtgttatcactc
atggttatggcagcactgcataattctcttactgtcatgccatccgtaagatgcttttctgtgactggtg
agtactcaaccaagtcattctgagaatagtgtatgcggcgaccgagttgctcttgcccggcgtcaatacg
ggataataccgcgccacatagcagaactttaaaagtgctcatcattggaaaacgttcttcggggcgaaaa
ctctcaaggatcttaccgctgttgagatccagttcgatgtaacccactcgtgcacccaactgatcttcag
catcttttactttcaccagcgtttctgggtgagcaaaaacaggaaggcaaaatgccgcaaaaaagggaat
aagggcgacacggaaatgttgaatactcatactcttcctttttcaatattattgaagcatttatcagggt
tattgtctcatgagcggatacatatttgaatgtatttagaaaaataaacaaataggggttccgcgcacat
ttccccgaaaagtgccacctgac

What is claimed is:

1. A composition for epigenome modification of a SNCA gene, the composition comprising:
(a) (i) a fusion protein or (ii) a nucleic acid sequence encoding a fusion protein, the fusion protein comprising two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, deacetylase activity, or combination thereof, and
(b) (i) at least one guide RNA (gRNA) or (ii) a nucleic acid sequence encoding at least one guide gRNA, wherein the at least one gRNA targets the fusion protein to a target region within the SNCA gene.

2. The composition of claim 1, wherein the at least one gRNA targets the fusion protein to a target region within intron 1 of the SNCA gene.

3. The composition of claim 2, wherein the composition modifies at least one CpG island region within intron 1 of the SNCA gene.

4. The composition of claim 3, wherein the at least one CpG island region comprises CpG1, CpG2, CpG3, CpG4, CpG5, CpG6, CpG7, CpG8, CpG9, CpG10, CpG11, CpG12, CpG13, CpG14, CpG15, CpG16, CpG17, CpG18, CpG19, CpG20, CpG21, CpG22, CpG23, or a combination thereof.

5. The composition of claim 3 or 4, wherein the at least one CpG island region comprises CpG1, CpG3, CpG6, CpG7, CpG8, CpG9, CpG18, CpG19, CpG20, CpG21, CpG22, or a combination thereof.

6. The composition of any one of claims 3-5, wherein the second polypeptide domain comprises a peptide having methylase activity and the fusion protein methylates at least one CpG island region within intron 1 of the SNCA gene.

7. The composition of any one of claims 1-6, wherein the at least one gRNA comprises a polynucleotide sequence of at least one of SEQ ID NO: 2, SEQ ID NO: 3, SEQ ID NO: 4, SEQ ID NO: 5, complement thereof, variant thereof, or a combination thereof.

8. The composition of claim 1, wherein the at least one gRNA targets the fusion protein to a target region within intron 4 of the SNCA gene, and optionally, wherein the target region within intron 4 is a H3K4Me3, H3K4Me1 and/or H3K27Ac mark.

9. The composition of any one of claims 1-8, wherein the second polypeptide domain comprises DNA (cytosine-5)-methyltransferase 3A (DNMT3A), a functional fragment thereof, and/or a variant thereof.

10. The composition of any one of claims 1-9, wherein the fusion protein represses the transcription of the SNCA gene.

11. The composition of any one of claims 1-10, wherein the Cas protein comprises a Cas9 endonuclease having at least one amino acid mutation which knocks out nuclease activity of Cas9.

12. The composition of claim 11, wherein the at least one amino acid mutation is at least one of D10A and H840A.

13. The composition of claim 11 or 12, wherein the Cas protein comprises an amino acid sequence of SEQ ID NO: 10.

14. The composition of any one of claims 1-13, wherein the second polypeptide domain is fused to the C-terminus, N-terminus, or both, of the first polypeptide domain.

15. The composition of any one of claims 1-14, further comprising a nuclear localization sequence.

16. The composition of any one of claims 1-15, further comprising a linker connecting the first polypeptide domain to the second polypeptide domain.

17. The composition of any one of claims 1-16, wherein the second polypeptide domain comprises an amino acid sequence of SEQ ID NO: 11.

18. The composition of any one of claims 1-17, wherein the fusion protein comprises an amino acid sequence of SEQ ID NO: 13.

19. The composition of any one of claims 1-18, wherein the fusion protein is encoded by a polynucleotide sequence comprising a polynucleotide sequence of SEQ ID NO: 14.

20. The composition of any one of claims 1-19, comprising administering to, or provided in, the subject any of: (a)(ii) and (b)(ii), (a)(i) and (b)(i), (a)(i) and (b)(ii), or (a)(ii) and (b)(i).

21. The composition of any one of claims 1-20, wherein the nucleic acid of (a)(ii) and/or (b)(ii) comprises DNA or RNA.

22. The composition of any one of claims 1-21, wherein one or both of (a) and (b) are packaged in a viral vector.

23. The composition of any one of claims 1-22, wherein (a) and (b) are packaged in the same viral vector.

24. The composition of claim 22 or 23, wherein the viral vector comprises a lentiviral vector.

25. The composition of any one of claims 22-24, wherein the viral vector comprises an episomal integrase-deficient lentiviral vector (IDLV) or an episomal integrase-competent lentiviral vector (ICLV).

26. The composition of any one of claims 22-25, wherein the viral vector comprises a polycistronic-protein composition comprising multiple promoters, p2a; t2a; IRES, or combinations thereof.

27. An isolated polynucleotide encoding the composition of any one of claims 1-26.

28. A vector comprising the isolated polynucleotide of claim 27.

29. The vector of claim 28, wherein the vector is a viral vector.

30. The vector of claim 28 or 29, wherein the viral vector is a lentiviral vector.

31. The vector of any one of claims 28-30, wherein the viral vector is an episomal integrase-deficient lentiviral vector (IDLV) or an episomal integrase-competent lentiviral vector (ICLV).

32. A host cell comprising the isolated polynucleotide of claim 27 or the vector of any one of claims 28-31.

33. A pharmaceutical composition comprising at least one of the composition of claims 1-26, the isolated polynucleotide of claim 27, the vector of any one of claims 28-31, the host cell of claim 32, or combinations thereof.

34. A kit comprising at least one of the composition of claims 1-26, the isolated polynucleotide of claim 27, the vector of any one of claims 28-31, or combinations thereof.

35. A method of in vivo modulation of expression of a SNCA gene in a cell or a subject, the method comprising contacting the cell or subject with at least one of the composition of claims 1-26, the isolated polynucleotide of claim 27, the vector of any one of claims 28-31, the pharmaceutical composition of claim 33, or combinations thereof, in an amount sufficient to modulate expression of the gene.

36. A method of treating a disease or disorder associated with elevated SNCA expression levels in a subject, the method comprising administering to the subject or a cell in the subject at least one of the composition of claims 1-26, the isolated polynucleotide of claim 27, the vector of any one of claims 28-31, the pharmaceutical composition of claim 33, or combinations thereof.

37. A method of in vivo modulating expression of a SNCA gene in a cell or a subject, the method comprising contacting the cell or subject with:
(a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and
(b)(i) at least one guide RNA (gRNA) that targets the fusion molecule to a target region within the SNCA gene or (b)(ii) a nucleic acid sequence encoding at least one gRNA that targets the fusion protein to a target region within the SNCA gene,
in an amount sufficient to modulate expression of the gene.

38. A method of treating a disease or disorder associated with elevated SNCA expression levels in a subject, the method comprising administering to the subject or a cell in the subject:
(a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and
(b)(i) at least one guide RNA (gRNA) that targets the fusion molecule to a target region within the SNCA gene or (b)(ii) a nucleic acid sequence encoding at least one gRNA that targets the fusion molecule to a target region within the SNCA gene,
in an amount sufficient to modulate expression of the gene.

39. The method of claim 37 or 38, wherein the at least one gRNA or nucleic acid sequence encoding the at least one gRNA targets the fusion protein to a target region within intron 1 of the SNCA gene.

40. The method of claim 39, wherein the fusion protein modifies at least one CpG island region within intron 1 of the SNCA gene.

41. The method of claim 40, wherein the at least one CpG island region comprises CpG1, CpG2, CpG3, CpG4, CpG5, CpG6, CpG7, CpG8, CpG9, CpG10, CpG11, CpG12, CpG13, CpG14, CpG15, CpG16, CpG17, CpG18, CpG19, CpG20, CpG21, CpG22, CpG23, or a combination thereof.

42. The method of claim 40 or 41, wherein the at least one CpG island region comprises CpG1, CpG3, CpG6, CpG7, CpG8, CpG9, CpG18, CpG19, CpG20, CpG21, CpG22, or a combination thereof.

43. The method of any one of claims 40-42, wherein the second polypeptide domain comprises a peptide having methylase activity and the fusion protein methylates at least one CpG island region within intron 1 of the SNCA gene.

44. The method of any one of claims 37-43, wherein the at least one gRNA comprises a polynucleotide sequence of at least one of SEQ ID NO: 2, SEQ ID NO: 3, SEQ ID NO: 4, SEQ ID NO: 5, complement thereof, variant thereof, or a combination thereof.

45. The method of claim 37 or 38, wherein the at least one gRNA or nucleic acid sequence encoding the at least one gRNA targets the fusion protein to a target region within intron 4 of the SNCA gene, and optionally, wherein the target region within intron 4 is a H3K4Me3, H3K4Me1 and/or H3K27Ac mark.

46. The method of any one of claims 37-45, wherein the second polypeptide domain comprises DNA (cytosine-5)-methyltransferase 3A (DNMT3A), a functional fragment thereof, and/or a variant thereof.

47. The method of any one of claims 37-46, wherein the fusion protein represses the transcription of the SNCA gene.

48. The method of any one of claims 37-47, wherein the Cas protein comprises a Cas9 endonuclease having at least one amino acid mutation which knocks out nuclease activity of Cas9.

49. The method of claim 48, wherein the at least one amino acid mutation is at least one of D10A and H840A.

50. The method of claim 48 or 49, wherein the Cas protein comprises an amino acid sequence of SEQ ID NO: 10.

51. The method of any one of claims 37-50, wherein the second polypeptide domain is fused to the C-terminus, N-terminus, or both, of the first polypeptide domain.

52. The method of any one of claims 37-51, further comprising a nuclear localization sequence.

53. The method of any one of claims 37-52, further comprising a linker connecting the first polypeptide domain to the second polypeptide domain.

54. The method of any one of claims 37-53, wherein the second polypeptide domain comprises an amino acid sequence of SEQ ID NO: 11.

55. The method of any one of claims 37-54, wherein the fusion protein comprises an amino acid sequence of SEQ ID NO: 13.

56. The method of any one of claims 37-55, wherein the fusion protein is encoded by a polynucleotide sequence comprising a polynucleotide sequence of SEQ ID NO: 14.

57. The method of any one of claims 37-56, comprising administering to, or provided in, the subject any of: (a)(ii) and (b)(ii), (a)(i) and (b)(i), (a)(i) and (b)(ii), or (a)(ii) and (b)(i).

58. The method of any one of claims 37-57, wherein the nucleic acid of (a)(ii) and/or (b)(ii) comprises DNA or RNA.

59. The method of any one of claims 37-58, wherein one or both of (a) and (b) are packaged in a viral vector.

60. The method of any one of claims 37-59, wherein (a) and (b) are packaged in the same viral vector.

61. The method of claim 59 or 60, wherein the viral vector comprises a lentiviral vector.

62. The method of any one of claims 59-61, wherein the viral vector comprises an episomal integrase-deficient lentiviral vector (IDLV) or an episomal integrase-competent lentiviral vector (ICLV).

63. The method of any one of claims 35-62, wherein the cell comprises SNCA gene triplication (SNCA-Tri), wherein the levels of SNCA are elevated compared to physiological levels in a control cell that does not have SNCA-Tri.

64. The method of claim 63, wherein the SNCA levels are reduced to physiological levels after administering or providing any one of (a)(ii) and (b)(ii), (a)(i) and (b)(i), (a)(i) and (b)(ii), or (a)(ii) and (b)(i) to the subject or cell in the subject.

65. The method of any one of claims 35-64, wherein the expression of the SNCA gene is reduced by at least 20%.

66. The method of any one of claims 35-65, wherein the expression of the SNCA gene is reduced by at least 90%.

67. The method of any one of claims 35-66, wherein levels of α-synuclein are reduced by at least 25%.

68. The method of any one of claims 35-67, wherein levels of α-synuclein are reduced by at least 36%.

69. The method of any one of claims 35-68, wherein mitochondrial superoxide production is reduced by at least 25% and/or cell viability is increased at least 1.4 fold.

70. The method of any one of claims 36 or 38-69, wherein the disease or disorder is a neurodegenerative disorder.

71. The method of claim 70, wherein the neurodegenerative disorder is a SNCA-related disease or disorder.

72. The method of claim 70 or 71, wherein the neurodegenerative disorder is a synucleinopathy.

73. The method of any one of claims 70-72, wherein the neurodegenerative disorder is Parkinson's disease or dementia with Lewy bodies.

74. The method of any one of claims 35-73, wherein the cell is a dopaminergic (ventral midbrain) Neural Progenitor Cell (MD NPC), a midbrain dopaminergic neuron (mDA) or a basal forebrain cholinergic neuron (BFCN).

75. The method of any one of claims 35-74, wherein the subject is a mammal.

76. The method of any one of claims 35-75, wherein the subject is a human or a murine subject.

77. The method of any one of claims 35-76, wherein the viral vector comprises a polycistronic-protein composition comprising multiple promoters, p2a; t2a; IRES, or combinations thereof.

78. A viral vector system for epigenemic editing, the viral vector system comprising:
(a) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and
(b) a nucleic acid sequence encoding at least one guide RNA (gRNA) that targets the fusion protein to a target region within the SNCA gene.

79. The viral vector system of claim 78, wherein the at least one gRNA targets the fusion protein to a target region within intron 1 of the SNCA gene.

80. The viral vector system of claim 79, wherein the fusion protein modifies at least one CpG island region within intron 1 of the SNCA gene.

81. The viral vector system of claim 80, wherein the at least one CpG island region comprises CpG1, CpG2, CpG3, CpG4, CpG5, CpG6, CpG7, CpG8, CpG9, CpG10, CpG11, CpG12, CpG13, CpG14, CpG15, CpG16, CpG17, CpG18, CpG19, CpG20, CpG21, CpG22, CpG23, or a combination thereof.

82. The viral vector system of claim 80 or 81, wherein the at least one CpG island region comprises CpG1, CpG3, CpG6, CpG7, CpG8, CpG9, CpG18, CpG19, CpG20, CpG21, CpG22, or a combination thereof.

83. The viral vector system of any one of claims 80-82, wherein the second polypeptide domain comprises a peptide having methylase activity and the fusion protein methylates at least one CpG island region within intron 1 of the SNCA gene.

84. The viral vector system of any one of claims 78-83, wherein the at least one gRNA comprises a polynucleotide sequence of at least one of SEQ ID NO: 2, SEQ ID NO: 3, SEQ ID NO: 4, SEQ ID NO: 5, complement thereof, variant thereof, or a combination thereof.

85. The viral vector system of claim 78, wherein the at least one gRNA targets the fusion protein to a target region within intron 4 of the SNCA gene, and optionally, wherein the target region within intron 4 is a H3K4Me3, H3K4Me1 and/or H3K27Ac mark.

86. The viral vector system of any one of claims 78-85, wherein the second polypeptide domain comprises DNA (cytosine-5)-methyltransferase 3A (DNMT3A), a functional fragment thereof, and/or a variant thereof.

87. The viral vector system of any one of claims 78-86, wherein the second polypeptide domain comprises an amino acid sequence of SEQ ID NO:11.

88. The viral vector system of any one of claims 78-87, wherein the Cas protein comprises a Cas9 endonuclease having at least one amino acid mutation which knocks out nuclease activity of Cas9.

89. The viral vector system of claim 88, wherein the at least one amino acid mutation is at least one of D10A and H840A.

90. The viral vector system of claim 88 or 89, wherein the Cas protein comprises an amino acid sequence of SEQ ID NO: 10.

91. The viral vector system of any one of claims 78-90, wherein the second polypeptide domain is fused to the C-terminus, N-terminus, or both, of the first polypeptide domain.

92. The viral vector system of any one of claims 78-91, further comprising a nuclear localization sequence.

93. The viral vector system of any one of claims 78-92, further comprising a linker connecting the first polypeptide domain to the second polypeptide domain.

94. The viral vector system of any one of claims 78-93, wherein the fusion protein comprises an amino acid sequence of SEQ ID NO: 13.

95. The viral vector system of any one of claims 78-94, wherein the fusion protein is encoded by a polynucleotide sequence comprising a polynucleotide sequence of SEQ ID NO: 14.

96. The viral vector system of any one of claims 78-95, wherein the viral vector is a lentiviral vector.

97. The viral vector system of any one of claims 78-96, wherein the viral vector is an episomal integrase-deficient lentiviral vector (IDLV) or an episomal integrase-competent lentiviral vector (ICLV).

98. A method of reversing DNA damage in a subject suffering from a disease or disorder associated with elevated SNCA expression levels, the method comprising contacting the cell or subject with at least one of the composition of claims 1-26, the isolated polynucleotide of claim 27, the vector of any one of claims 28-31, the pharmaceutical composition of claim 33, or combinations thereof, in an amount sufficient to modulate expression of the gene.

99. A method of rescuing aging-related abnormal nuclei in a subject suffering from a disease or disorder associated with elevated SNCA expression levels, the method comprising contacting the cell or subject with at least one of the composition of claims 1-26, the isolated polynucleotide of claim 27, the vector of any one of claims 28-31, the pharmaceutical composition of claim 33, or combinations thereof, in an amount sufficient to modulate expression of the gene.

100. A method of increasing nuclear circularity or decreasing folded nuclei in a subject suffering from a disease or disorder associated with elevated SNCA expression levels, the method comprising contacting the cell or subject with at least one of the composition of claims 1-26, the isolated polynucleotide of claim 27, the vector of any one of claims 28-31, the pharmaceutical composition of claim 33, or combinations thereof, in an amount sufficient to modulate expression of the gene.

101. A method of reversing DNA damage in a subject suffering from a disease or disorder associated with elevated SNCA expression levels, the method comprising contacting the cell or subject with: (a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b)(i) at least one guide RNA (gRNA) that targets the fusion molecule to a target region within the SNCA gene or (b)(ii) a nucleic acid sequence encoding at least one gRNA that targets the fusion protein to a target region within the SNCA gene, in an amount sufficient to modulate expression of the gene.

102. A method of rescuing aging-related abnormal nuclei in a subject suffering from a disease or disorder associated with elevated SNCA expression levels, the method comprising contacting the cell or subject with: (a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b)(i) at least one guide RNA (gRNA) that targets the fusion molecule to a target region within the SNCA gene or (b)(ii) a nucleic acid sequence encoding at least one gRNA that targets the fusion protein to a target region within the SNCA gene, in an amount sufficient to modulate expression of the gene.

103. A method of increasing nuclear circularity or decreasing folded nuclei in a subject suffering from a disease or disorder associated with elevated SNCA expression levels, the method comprising contacting the cell or subject with: (a)(i) a fusion protein or (a)(ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b)(i) at least one guide RNA (gRNA) that targets the fusion molecule to a target region within the SNCA gene or (b)(ii) a nucleic acid sequence encoding at least one gRNA that targets the fusion protein to a target region within the SNCA gene, in an amount sufficient to modulate expression of the gene.

104. The composition of any one of claims 22-26, wherein the viral vector comprises a polynucleotide sequence of SEQ ID NO: 38, SEQ ID NO: 41, SEQ ID NO: 40, or SEQ ID NO: 39.

105. The vector of any one of claims 28-31, wherein the viral vector comprises a polynucleotide sequence of SEQ ID NO: 38, SEQ ID NO: 41, SEQ ID NO: 40, or SEQ ID NO: 39.

106. The method of any one of claims 59-62, wherein the viral vector comprises a polynucleotide sequence of SEQ ID NO: 38, SEQ ID NO: 41, SEQ ID NO: 40, or SEQ ID NO: 39.

107. The viral vector system of any one of claims 78-97, wherein the viral vector comprises a polynucleotide sequence of SEQ ID NO: 38, SEQ ID NO: 41, SEQ ID NO: 40, or SEQ ID NO: 39.


SEQUENCE LISTING (42 sequences)

SEQ ID NO: 1 - DNA, 472 bp, Artificial Sequence
  Synthetic
gggaggtgagtacttgtccctttggggagcctaaggaaagagacttgacctggctttcgtcctgcttctgatattcccttctccacaagggctgagagattaggctgcttctccgggatccgcttttccccgggaaacgcgaggatgctccatggagcgtgagcatccaacttttctctcacataaaatctgtctgcccgctctcttggtttttctctgtaaagtaagcaagctgcgtttggcaaataatgaaatggaagtgcaaggaggccaagtcaacaggtggtaacgggttaacaagtgctggcgcggggtccgctagggtggaggctgagaacgccccctcgggtggctggcgcggggttggagacggcccgcgagtgtgagcggcgcctgctcagggtagatagctgagggcgggggtggatgttggatggattagaaccatcacacttgggcctgctgtttgcct

SEQ ID NO: 2 - DNA, 20 bp, Artificial Sequence
  Synthetic
ttgtccctttggggagccta

SEQ ID NO: 3 - DNA, 20 bp, Artificial Sequence
  Synthetic
aataatgaaatggaagtgca

SEQ ID NO: 4 - DNA, 20 bp, Artificial Sequence
  Synthetic
ggaggctgagaacgccccct

SEQ ID NO: 5 - DNA, 20 bp, Artificial Sequence
  Synthetic
ctgctcagggtagatagctg

SEQ ID NO: 6 - DNA, 23 bp, Artificial Sequence
  Synthetic
ttgtccctttggggagcctaagg

SEQ ID NO: 7 - DNA, 23 bp, Artificial Sequence
  Synthetic
aataatgaaatggaagtgcaagg

SEQ ID NO: 8 - DNA, 23 bp, Artificial Sequence
  Synthetic
ggaggctgagaacgccccctcgg

SEQ ID NO: 9 - DNA, 23 bp, Artificial Sequence
  Synthetic
ctgctcagggtagatagctgagg

SEQ ID NO: 10 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 11 - protein, 311 aa, Artificial Sequence
  Synthetic
PSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 12 - DNA, 933 bp, Artificial Sequence
  Synthetic
ccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtg

SEQ ID NO: 13 - protein, 1702 aa, Artificial Sequence
  Synthetic
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKLEGGGGSGSPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFAC

SEQ ID NO: 14 - DNA, 5109 bp, Artificial Sequence
  Synthetic
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtg

SEQ ID NO: 15 - DNA, 18 bp, Artificial Sequence
  Synthetic
gagcggatccccctcccg

SEQ ID NO: 16 - DNA, 20 bp, Artificial Sequence
  Synthetic
ctctccactgccggatccgg

SEQ ID NO: 17 - DNA, 23 bp, Artificial Sequence
  Synthetic
tttttggggagtttaaggaaaga

SEQ ID NO: 18 - DNA, 24 bp, Artificial Sequence
  Synthetic
aacctccttacacttccatttcat

SEQ ID NO: 19 - DNA, 18 bp, Artificial Sequence
  Synthetic
ggggagtttaaggaaaga

SEQ ID NO: 20 - DNA, 24 bp, Artificial Sequence
  Synthetic
tggggagtttaaggaaagagattt

SEQ ID NO: 21 - DNA, 24 bp, Artificial Sequence
  Synthetic
acctccttacacttccatttcatt

SEQ ID NO: 22 - DNA, 20 bp, Artificial Sequence
  Synthetic
ggttgagagattaggttgtt

SEQ ID NO: 23 - DNA, 23 bp, Artificial Sequence
  Synthetic
ttggggagtttaaggaaagagat

SEQ ID NO: 24 - DNA, 24 bp, Artificial Sequence
  Synthetic
acctccttacacttccatttcatt

SEQ ID NO: 25 - DNA, 17 bp, Artificial Sequence
  Synthetic
agagaggatgttttatg

SEQ ID NO: 26 - DNA, 23 bp, Artificial Sequence
  Synthetic
tttttggggagtttaaggaaaga

SEQ ID NO: 27 - DNA, 23 bp, Artificial Sequence
  Synthetic
cctccttacacttccatttcatt

SEQ ID NO: 28 - DNA, 21 bp, Artificial Sequence
  Synthetic
cttacacttccatttcattat

SEQ ID NO: 29 - DNA, 24 bp, Artificial Sequence
  Synthetic
tggggagtttaaggaaagagattt

SEQ ID NO: 30 - DNA, 23 bp, Artificial Sequence
  Synthetic
ccctcaactatctaccctaaaca

SEQ ID NO: 31 - DNA, 19 bp, Artificial Sequence
  Synthetic
gagtttggtaaataatgaa

SEQ ID NO: 32 - DNA, 24 bp, Artificial Sequence
  Synthetic
gtgtaaggaggttaagttaatagg

SEQ ID NO: 33 - DNA, 29 bp, Artificial Sequence
  Synthetic
acaacaaacccaaatataataattctaat

SEQ ID NO: 34 - DNA, 22 bp, Artificial Sequence
  Synthetic
aggttaagttaataggtggtaa

SEQ ID NO: 35 - DNA, 23 bp, Artificial Sequence
  Synthetic
tttttggggagtttaaggaaaga

SEQ ID NO: 36 - DNA, 24 bp, Artificial Sequence
  Synthetic
ctcaaacaaacaacaaacccaaat

SEQ ID NO: 37 - DNA, 24 bp, Artificial Sequence
  Synthetic
ctcaaacaaacaacaaacccaaat

SEQ ID NO: 38 - DNA, 13039 bp, Artificial Sequence
  Synthetic
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgctcagggtagatagctggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 39 - DNA, 14092 bp, Artificial Sequence
  Synthetic
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgtgtacacgtctctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 40 - DNA, 13812 bp, Artificial Sequence
  Synthetic
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgtgtacacgtctctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggggccggcccggatccggcgcaacaaacttctctctgctgaaacaagccggagatgtcgaagagaatcctggaccgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtgccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 41 - DNA, 13813 bp, Artificial Sequence
  Synthetic
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtttttcaagcggaaacgctagttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggggccggcccggatccggcgcaacaaacttctctctgctgaaacaagccggagatgtcgaagagaatcctggaccgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtgccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 42 - DNA, 20 bp, Artificial Sequence
  Synthetic
tttttcaagcggaaacgcta